(19) United States
(12) Patent Application Publication
(10) Pub. No.: US 2003/0235816 A1
(43) Pub. Date: Dec. 25, 2003

(54) Method to determine outcome for patients with prostatic disease

(75) Inventors: Kevin M. Slawin, Houston, TX (US); Shahrokh Shariat, Dallas, TX (US); Michael Kattan, New York, NY (US); Peter T. Scardino, Manhattan, NY (US)

Correspondence Address: SCHWEGMAN, LUNDBERG, WOESSNER & KLUTH, P.A., P.O. BOX 2938, MINNEAPOLIS, MN, 55402, US

(73) Assignee: Baylor College of Medicine (by Slawin and Shariat)

(21) Appl. No.: 10/389,245

(22) Filed: Mar. 14, 2003

Related U.S. Application Data
(60) Provisional application No. 60/364,658, filed on Mar. 14, 2002.
(60) Provisional application No. 60/412,085, filed on Sep. 18, 2002.

(51) Int. Cl. C12Q 1/70

(52) U.S. Cl. 435/5

(57) ABSTRACT

A method for prognosis of patients with prostate cancer, e.g., clinically localized prostate cancer, is provided.

### CROSS-REFERENCE TO RELATED APPLICATIONS

This application claims the benefit of the filing date of U.S. application Serial No. 60/364,658, filed Mar. 14, 2002, and of U.S. application Serial No. 60/412,085, filed Sep. 18, 2002, the disclosures of which are incorporated by reference herein.

### STATEMENT OF GOVERNMENT RIGHTS

The invention was made at least in part with a grant from the Government of the United States of America (grant no. CA 58203 from the National Institutes of Health). The Government has certain rights to the invention.

### BACKGROUND OF THE INVENTION

Prostate cancer is the most commonly diagnosed cancer and the second leading cause of cancer death for men in the United States. In 1999, an estimated 179,300 men were diagnosed with prostate cancer and 37,000 died of this disease. Despite the identification of several new potential biomarkers for prostate cancer (e.g., p53, p21, p27, and E-cadherin), prostate specific antigen (PSA) and the histologic Gleason score have remained the most commonly used predictors of prostate cancer biology. In fact, the widespread use of PSA-based screening has dramatically increased the number of men diagnosed and treated for clinically localized prostate cancer over the past decade. Concomitantly the incidence of clinical metastatic disease at the time of initial diagnosis has dropped considerably, in concert with an overall decrease in prostate cancer mortality (Merill et al., 2000).

Even given the significant rate of long-term cancer control afforded patients with clinically localized prostate cancer treated with radical prostatectomy or radiation therapy, approximately 30% of these patients will fail treatment, as evidenced by a detectable or rising PSA, which often is due to early dissemination of microscopic metastatic disease prior to primary therapy (Pound et al., 1997). Conventional staging modalities such as bone scan, CT scan, and MRI have a limited role in staging patients with clinically localized prostate cancer, because of their poor performance in detecting early, low-volume metastases (Oesterling et al., 1993; Engeler et al., 1992). Pre-operative nomograms that consider established markers such as PSA, clinical stage, and biopsy Gleason score can provide an estimate of the risk of nodal metastasis or disease recurrence, but are still imperfect for determining the pathological stage or prognosis in individual patients (Partin et al., 1997; Kattan et al., 1998). Improved pre-operative identification of patients with occult metastatic disease, who have a high probability of developing disease progression despite effective local therapy, would be helpful in sparing men from the morbidity of a radical prostatectomy or radiation therapy that would be ineffective or for selecting patients best suited for clinical trials of neoadjuvant or adjuvant therapy.

One example of a molecule which has been investigated for its association with cancer is transforming growth factor β₁ (TGF-β₁), a pleiotropic growth factor that regulates cellular proliferation, chemotaxis, cellular differentiation, immune response, and angiogenesis. Loss of response to the inhibitory effect of TGF-β₁ has been associated with the progression of cancer. For example, increased local expression of TGF-β₁ has been associated with tumor grade, pathological stage, and lymph node metastasis in patients with prostate cancer (Steiner et al., 1992; Eastham et al., 1995; Truong et al., 1993; Thompson et al., 1992). In addition, elevated circulating levels of TGF-β₁ have been found in patients with a variety of different tumors (Wakefield et al, 1995; Kong et al., 1999; Shirai et al., 1994; Eder et al., 1996; Junker et al., 1996). Although higher circulating TGF-β₁ levels have shown an association with prostate cancer invasion (Ivanovic et al., 1995) and metastasis in some studies (Ivanovic et al., 1995; Adler et al., 1999; Kakehi et al, 1996), other studies have not shown such an association (Wolff et al., 1999; Perry et al., 1997). Thus, it is unclear whether circulating TGF-β₁ levels are associated with prostate cancer invasion and metastases.

Insulin-like growth factors (IGFs) are potent mitogens that enhance cell growth and proliferation. Paracrine stimulation of the IGF-I signaling pathway has been implicated in the progression of prostate cancer. IGF binding proteins (IGF BPs) function indirectly by regulating IGF bioavailability, but also have direct IGF-independent effects. Increased circulating levels of IGF BP-2 have been observed in prostate cancer and low IGF BP-3 levels have been associated with increased prostate cancer risk, however, the relative importance of systemic levels of IGFs and IGF BPs in prostate cancer remains unclear.

Interleukin-6 (IL-6) is a molecule that regulates the growth and differentiation of various types of malignant tumors, including prostate carcinomas. Circulating levels of IL-6 have been shown to be elevated in patients with locally advanced and metastatic prostate cancer. IL-6 signaling occurs through a receptor complex consisting of a specific receptor and a signal-transducing component (gp130). The soluble form of the IL-6 receptor (IL-6sR), which arises from proteolytic cleavage of membrane-bound IL-6 receptor, can augment IL-6 induced signaling by facilitating the binding of the IL-6/IL-6sR complex to membrane-bound gp130.

Angiogenesis plays a central role in prostate tumor growth and metastasis. Data from transgenic mouse models as well as from a variety of human tumors suggest that the switch to an angiogenic phenotype occurs relatively early during the tumor growth and progression (Weidner et al., 1991; Macleod et al., 1999). In prostate cancer, the conversion to an angiogenic phenotype has been associated with tumorigenesis (Ali et al., 2000; Huss et al., 2001) and late stages of tumor progression (Volavsek et al., 2000; Garcia et al., 2000). Tumor angiogenesis as evaluated by immunohistochemical microvessel density has been associated with clinical and pathologic features of biologically aggressive prostate cancer, disease progression and metastasis (Weidner et al. 1993; Bostwick et al., 1996; Silberman et al., 1997; Mehta et al., 2001).

Immunohistochemistry requires removal of the tumor and counting of microvessel density after staining with antibodies to endothelial cell antigens. Even with use of sophisticated computerized imaging systems, this technique is labor-intensive. In addition, differences in antibodies, varying interpretation and stratification criteria, specimen handling, and technical procedure limit the use of immunohistochemical assessment of angiogenesis in a clinical setting. Moreover, circulating tumors cells are thought to promote their own metastasis via interaction with endothelial cells by intravasation and extravasation, however, the mechanism remains unclear.

VEGF is a homodimeric, heparin-binding glycoprotein that is produced by almost every cell type. The VEGFs are a family of related proteins, six of which have been identified to date. The VEGFs modulate their activities through several receptors. VEGF, the parent compound has multiple and diverse functions including promotion of endothelial cell mitogenesis and survival (anti-apoptotic effects), chemotactic effects, increased vascular permeability, immune effects via inhibition of maturation of antigen-presenting dendritic cells, and vasodilatation. Normal prostate epithelial cells as well as malignant prostate tissue have been have been shown to constitutively express VEGF (Benjamin et al., 1999), however, other studies have shown that compared to tissue derived from benign prostate hyperplasias, malignant prostate tissue produces significantly higher levels of VEGF (Ferrer et al., 1998). Plasma levels of VEGF have been reported to be increased in patients with metastatic prostate cancer (Duque et al., 1999). In addition, higher pre-treatment plasma VEGF levels have been demonstrated to be independently associated with decreased survival in hormone-refractory prostate cancer patients (George et al., 2001).

VCAM-1 is a 90-kd transmembrane glycoprotein that is expressed transiently on activated vascular endothelial cells in response to vascular endothelial growth factors and other cytokines. Inflammatory cells often surround tumors, which produce cytokines. Endothelial expression of VCAM-1 plays a major role in adhesion of leukocytes to the endothelium in inflammation. However, cellular adhesion markers are not only involved in inflammation but also in tumor metastasis (Zetter, 1993). TNF-α, a cytokine known to be implicated in prostate stroma-epithelium interaction, has been shown to increase VCAM in tumor cells by two-fold (Simiantonaki et al., 2002) and also in prostate cancer (Cooper et al., 2002). In addition, endothelial cells expressing VCAM-1 bind melanoma cell lines, suggesting that VCAM-1 may function as an adhesion molecule to facilitate metastasis (Langley et al., 2001). The elevated local expression of VCAM-1 has been associated with advanced pathological stage in prostate cancer patients (Wikstrom et al., 2002).

VCAM-1 is also released in a soluble form. Serum soluble VCAM-1 (sVCAM-1) has been shown to correlate closely with microvessel density in tumor specimens and to be strongly associated with breast cancer stage, progression and response to hormone therapy (Byrne et al., 2000). In prostate cancer, serum level of sVCAM-1 was shown to not be clinically useful as a biomarker for differentiating prostate cancer from benign prostatic hyperplasia, for predicting progression, for identifying metastatic potential, or for monitoring treatment (Lynch et al., 1997). Although tumor invasiveness is likely mediated by cellular adhesion molecules and is necessary for initiation of metastasis, it cannot succeed without neo-vascularization through angiogenesis.

Recently, there has been a realization that pre-treatment PSA levels, the primary predictive parameter in the majority of tools to predict recurrence, may reflect primarily the presence of benign prostatic hyperplasia (BPH) rather than prostate cancer. Stamey et al. (2001) recently reported that for patients with a PSA level of ≦9 ng/mL, PSA poorly reflected the risk of progression after radical prostatectomy but was significantly correlated with the overall volume of the radical prostatectomy specimen; a direct reflection of the degree of BPH present. Several have failed to detect an independent predictive value for pre-operative PSA for disease progression in studies that have included more modern cohorts of patients with clinically localized prostate cancer undergoing radical prostatectomy who had lower median PSA levels than patients in most older studies.

While a number of molecules other than PSA are associated with prostate cancer, it is unclear whether any of these molecules, or which combinations of molecules, are useful to predict disease outcome. Therefore, there is an imminent need for nomograms that include novel markers that are specifically associated with biologically aggressive prostate cancer for improved prediction of outcome in patients with prostate-related disorders, such as patients diagnosed with clinically localized prostate cancer, and especially in those patients who are diagnosed with lower PSA levels.

### SUMMARY OF THE INVENTION

The invention provides methods, apparatus and nomograms to predict the status, e.g., disease-free status, of a prostate cancer patient after therapy, e.g., after radical prostatectomy, external beam radiation therapy, brachytherapy, or other localized therapies for prostate cancer, e.g., cryotherapy. The methods employ values or scores from biopsies, such as a 12 core biopsy set, prostatectomy final pathology, and/or other markers, e.g., markers present in a physiological fluid sample such as a protein found in the blood, to predict patient outcome. The biopsy or physiological fluid, e.g., blood sample, may be obtained from the patient prior to and/or after therapy for prostate cancer. When the sample is collected “after” therapy, it may be collected at times up to about 5 to 6 months, e.g., about 1, 2, 3, 4, or more months, e.g., 7, 8, 9, 10 or 11 months, after therapy, including from about 1, 2, 3, 4 or 5 days after therapy, up to about 1, 2, 3, 4, 5, or 6 weeks after therapy. In other embodiments, the sample may be collected years after therapy such as about 1, 2, 3, 4, 5, 6 or 7 years after therapy. In one embodiment, the sample is collected after therapy, for instance, at a time when PSA levels or amount are monitored or when PSA levels or amounts are rising over time.

In one embodiment, the invention includes correlating the value or score from various markers, such as protein markers, biopsy data, e.g., 12 core systematic biopsy data, and/or optionally prostatectomy final pathology, for example, in a nomogram, to predict, for instance, patient outcome, progression, risk of organ-confined disease, extracapsular extension, seminal vesicle invasion, and/or lymph node involvement. In another embodiment, the invention includes correlating the value or score from various markers, such as protein markers found in blood, biopsy data, e.g., 12 core systematic biopsy data, and/or optionally prostatectomy final pathology, from a patient with metastatic disease, either hormone sensitive or hormone refractory metastatic disease, to predict the aggressiveness of the disease and/or time to death.

For instance, the methods, apparatus or nomograms may be employed prior to localized therapy for prostate cancer, e.g., to predict risk of progression or predict organ-confined disease, after therapy for prostate cancer such as in patients with PSA recurrence, e.g., to predict aggressiveness of recurrence, time to metastasis and/or time to death, or, in patients with metastatic disease or hormone refractory metastatic disease, e.g., to predict the aggressiveness of disease and/or time to death.

In one embodiment of the invention, the method comprises contacting a physiological fluid sample from a patient prior to or after therapy for clinically localized prostate cancer with an agent that binds to TGF-β₁ so as to form a complex. Thus, in one embodiment of the invention, the method comprises contacting a physiological fluid sample from a patient after therapy for prostate cancer, e.g., a patient with clinically localized prostate cancer or having a clinical stage ≦T3a, with an agent that binds to TGF-β₁ so as to form a complex. The amount or level of complex formation is then correlated to the risk of non-prostate confined disease or disease progression in the patient. In one embodiment, the fluid sample is a blood sample and more preferably a plasma sample. In one embodiment, the sample is obtained from a patient that has not received any previous therapy for prostate cancer, e.g., hormonal therapy, radiation therapy or brachytherapy. Preferred agents that bind to TGF-β₁ include, but are not limited to, antibodies specific for TGF-β₁ and the TGF-β₁ receptor protein, e.g., type I or II. As used herein, a sample of “physiological body fluid” includes, but is not limited to, a sample of blood, plasma, serum, seminal fluid, urine, saliva, sputum, semen, pleural effusions, bladder washes, bronchioalveolar lavages, cerebrospinal fluid and the like. As used herein, a patient with “clinically localized prostate cancer” means that the patient has no clinically detectable metastases, e.g., detectable by MRI, bone scan, CT scan, or PET scan.

As described herein, the relationship between pre-operative or post-operative platelet-poor plasma TGF-β₁ levels and established markers of prostate cancer invasion, metastasis, and disease progression was determined in a large consecutive cohort of patients with prostate cancer, e.g., those undergoing radical prostatectomy. One study group consisted of 120 consecutive patients who underwent radical prostatectomy (median follow-up of 53.8 months) for clinically localized prostate cancer. Pre-operative platelet-poor plasma levels of TGF-β₁ were measured and correlated with clinical and pathological parameters. TGF-β₁ levels were also measured in 44 healthy men without any cancer, in 19 men with prostate cancer metastatic to regional lymph nodes, and in 10 men with prostate cancer metastatic to bone. None of the patients were treated with hormonal or radiation therapy prior to sample collection.

Plasma TGF-β₁ levels in patients with lymph node metastases (14.2±2.6 ng/mL) and bone metastases (15.5±2.4 ng/mL) were significantly higher than those in radical prostatectomy patients (5.2±1.3 ng/mL) and healthy subjects (4.5±1.2 ng/mL) (P values <0.001). Pre-operative plasma TGF-β₁ levels and biopsy Gleason grade were both significant independent predictors of organ-confined disease (P=0.006 and P=0.006, respectively) and PSA progression (P<0.001 and P=0.021, respectively). Within each pathological stage, patients who developed biochemical progression had significantly higher TGF-β₁ levels than those who remained disease-free after surgery (P values <0.001). In patients who progressed, pre-operative plasma TGF-β₁ levels were significantly higher in those with presumed distant versus local-only failure (P=0.019). In men without clinical or pathological evidence of metastases, pre-operative plasma TGF-β₁ levels were the strongest predictor of biochemical progression after surgery, likely due to an association with occult metastatic disease present at the time of radical prostatectomy.

Hence, the invention provides a method to determine the risk of progression of a patient after therapy for prostate cancer and/or the risk of non-prostate confined disease. The method comprises contacting a blood plasma sample obtained from a patient before therapy for prostate cancer, e.g., before a radical prostatectomy for clinically localized prostate cancer, with an agent that binds to TGF-β₁ so as to form a complex. Then the amount or level of complex formation is correlated with the risk of progression and/or the risk of non-prostate confined disease.

As also described herein, a larger cohort of 468 radical prostatectomy patients were employed to study marker interactions. Of these patients, 278 patients had samples available at 6 to 8 weeks after post-radical prostatectomy. The clinical stage of these patients was ≦T3a (47% cT1, 49% cT2, and 4% cT3a) and they had a median PSA of 8.2 ng/mL (range of 0.2 to 60 ng/mL). The median age for these patients was 63 years (range 40 to 81) and the median follow up for them was about 51 months. Fourteen percent (63/468) had PSA recurrence. Post-operative plasma TGF-β₁ levels were found to be useful as a prognostic marker for prostate cancer progression. Thus, serial measurements TGF-β₁ may be particularly useful to monitor the outcome of therapy, e.g., surgery, radiation, or hormonal therapy, or brachytherapy, similarly to serial measurements of PSA. Moreover, in a multivariate Cox proportional hazards model, post-therapy measurements of TGF-β₁ were found to be a stronger predictor than pre-therapy measurements of TGF-β₁ Accordingly, the invention provides a method to determine the risk of progression of a patient after therapy for prostate cancer. The method comprises contacting a blood plasma sample obtained from a patient after therapy for prostate cancer with an agent that binds to TGF-β₁ so as to form a complex.

Then the amount or level of complex formation is correlated with the risk of progression.

Thus, the level of TGF-β₁ in body fluids of humans is prognostically useful, and may optionally be employed in conjunction with other markers for neoplastic disease such as those for prostate cancer, e.g., urinary plasminogen activator (UPA), urinary plasminogen activator receptor (UPAR), plasminogen activator inhibitor 1 (PAI-1), IL-6, IL6sR, IGF BP-2, IGF BP-3, p53, Ki-67, p21, E-cadherin, and PSA, as well as VEGF, VCAM, e.g., sVCAM, Gleason scores and/or core data, e.g., in a nomogram to predict stage and/or outcome, e.g., the risk of organ-confined disease extracapsular extension, seminal vesicle invasion and/or lymph node involvement, in patients with prostate cancer. In one embodiment, the prognosis is based on a computer derived analysis of data of the amount, level or other value (score) for one or more markers for prostate cancer. Data may be input manually or obtained automatically from an apparatus for measuring the amount or level of one or more markers.

Thus, the invention provides a nomogram that may employ one or more standard clinical and pathological measures of prostate cancer, as well as one or more serum/plasma proteins, including, but not limited to, TGF-β₁, IL6, IL6sR, IGF BP-2, IGF BP-3, UPAR, UPA, PSA, VEGF and/or sVCAM, to predict outcomes in clinical situations for prostate cancer patients including pre-prostatectomy, post-prostatectomy, pre-radiation therapy, post-radiation therapy, recurrence after primary therapy, e.g., rising PSA after surgery or radiation therapy, and metastatic disease. In one embodiment, the method employs TGF-β₁, IL6sR and a Gleason score (grade), e.g., a primary Gleason score and/or a second Gleason score, and/or optionally clinical stage. Thus, in this embodiment of the invention, the method comprises providing, detecting or determining the amount or level of TGF-β₁ and IL6sR in a blood plasma sample, and a Gleason score from a sample comprising prostate cells, obtained from a patient prior to or after therapy for prostate cancer. Then the results are correlated to the risk of progression after therapy.

The invention also provides a prognostic method. The method comprises contacting a physiological fluid sample from a patient prior to or after primary therapy for clinically localized prostate cancer with an agent that binds to TGF-β₁ so as to form a complex. Then complex formation is detected or determined and the amount or level of complex formation is employed to predict the patient's final pathological stage and/or biochemical progression, e.g., after therapy or in the absence of therapy. Preferably, the sample is a blood sample, and more preferably, a plasma sample.

As also described herein, the pre-operative or post-operative plasma levels of IL-6 and IL6sR may be correlated with clinical and pathological parameters. Plasma IL-6 and IL6sR levels in patients with bone metastases were significantly higher than those in healthy subjects, in prostatectomy patients, or in patients with lymph node metastases (P values <0.001). In a pre-operative model that included IL-6 or IL6sR in addition to Partin nomogram variables, pre-operative plasma IL-6, IL6sR, and biopsy Gleason score were independent predictors of organ-confined disease (P values <0.01) and PSA progression (P values <0.028). However, in an alternative model that included both IL-6 and IL6sR, only pre-operative plasma IL6sR remained an independent predictor of PSA progression (P=0.038). Thus, IL-6 and IL6sR levels are elevated in men with prostate cancer metastatic to bone. In patients with clinically localized prostate cancer, the pre-operative plasma level of IL-6 and IL6sR are associated with markers of more aggressive prostate cancer and are predictors of biochemical progression after surgery.

Hence, the invention further provides a method in which a physiological fluid sample, e.g., blood serum or plasma, from a patient prior to or after primary therapy for clinically localized prostate cancer is contacted with an agent that binds to IL-6 or IL6sR so as to form a complex. Then the amount or level of complex formation is correlated to the risk of non-prostate confined disease (disease progression), final pathological stage and/or biochemical progression. Thus, the level of IL-6 and/or IL6sR in body fluids of humans is prognostically useful, and may optionally be employed in conjunction with other markers for neoplastic disease such as those for prostate cancer, e.g., UPA, UPAR, PAI-1, TGF-β₁, IGF BP-2, IGF BP-3, p53, p21, E-cadherin, and PSA, as well as VEGF, sVCAM, Gleason scores and/or core data, e.g., in a nomogram to predict stage and outcome in patients with prostate cancer. In one embodiment, the prognosis may be based on a computer derived analysis of data of the amount, level or other value for one or more markers for prostate cancer, and data may be input manually or obtained automatically.

In addition, pre- and post-operative TGF-β₁ levels were found to be significantly elevated in patients with advanced stage disease, including extraprostatic extension, seminal vesicle involvement, and metastases to lymph nodes. In contrast, pre-operative IL-6 and IL6sR levels were significantly associated with tumor volume, prostatectomy Gleason sum, and metastases to lymph nodes, but post-operative levels were not associated with any clinical or pathological parameters. In a post-operative model that includes pre- and post-operative TGF-β₁, IL-6, and IL6sR along with standard post-operative parameters, post-operative TGF-β₁ and prostatectomy Gleason sum were significant predictors of overall and aggressive disease progression. For all patients, plasma levels of all three markers declined significantly after prostate removal, while for patients that experienced disease progression, only IL-6 and IL6sR levels dropped significantly. Thus, for patients undergoing radical prostatectomy, pre-operative plasma levels of TGF-β₁ and IL6sR are associated with metastases to regional lymph nodes, presumed occult metastases at the time of primary treatment, and disease progression. After prostate removal, only post-operative TGF-β₁ level increases in value over pre-operative levels for the prediction of disease progression.

Accordingly, the invention provides a method to determine the risk of progression of a patient after therapy for prostate cancer. The method comprises contacting a blood plasma sample obtained from a patient before therapy for prostate cancer with an agent that binds to TGF-β₁ so as to form a complex, a blood plasma sample obtained from the patient after therapy for prostate cancer with an agent that binds to TGF-β₁ so as to form a complex, and a blood plasma sample obtained from the patient before therapy for prostate cancer with an agent that binds to IL6sR so as to form a complex. Then the amount or level of complex formation corresponding to pre-treatment and post-treatment TGF-β₁ levels and pre-treatment IL6sR levels is correlated with the risk of progression, e.g., in a nomogram.

As further described herein, pre-operative or post-operative plasma levels of IGF-1, IGF BP-2, and IGF BP-3 may be measured and correlated with clinical and pathological parameters. In the 120 patients, 44 healthy men without any cancer, 19 men with prostate cancer metastatic to regional lymph nodes, and the 10 men with prostate cancer metastatic to bone mentioned hereinabove, it was found that plasma IGF BP-2 levels in prostatectomy patients and in patients with lymph node metastases or bone metastases were significantly higher than those in healthy subjects (P values <0.006). Plasma IGBP-3 levels in patients with lymph node metastases and bone metastases were significantly lower than those in prostatectomy patients and healthy subjects (P values <0.031). Pre-operative plasma IGF BP-2 and biopsy Gleason score were both independent predictors of organ-confined disease (P=0.001 and P=0.005, respectively) and PSA progression (P=0.049 and P=0.035, respectively). When adjusted for IGF BP-2, IGF BP-3 was an independent predictor of PSA progression (P=0.040). Thus, while plasma IGF BP-2 levels are elevated in men with prostate cancer, IGF BP-3 levels are decreased in men with prostate cancer metastatic to regional lymph nodes and bone. In patients with clinically localized prostate cancer, the pre-operative plasma IGF BP-2 level is associated with markers of more aggressive prostate cancer and is a predictor of biochemical progression after surgery.

The invention thus provides a method which comprises contacting a physiological fluid sample, e.g., blood serum or plasma, from a patient prior to or after primary therapy for clinically localized prostate cancer with an agent that binds to IGF BP-2 and optionally to IGF BP-3, so as to form a complex. Complex formation is then detected or determined, and correlated to the risk of non-prostate confined disease), final pathological stage and/or biochemical progression. Similar to the methods described above, the level of IGF BP-2 and/or IGF BP-3 in body fluids of humans is prognostically useful, and may optionally be employed in conjunction with other markers for neoplastic disease such as those for prostate to predict stage and outcome in patients with prostate cancer, e.g., using a computer derived analysis of data of the amount, level or other value for one or more markers for prostate cancer.

As also described herein, levels of VEGF and sVCAM-1 were measured in plasma samples obtained pre-operatively from 215 patients undergoing radical prostatectomy for clinically localized disease and 9 men with untreated prostate cancer metastatic to bones. Plasma VEGF and sVCAM-1 levels were highest in patients with bone metastases (P<0.001). Within the group of prostatectomy patients, while pre-operative plasma VEGF and sVCAM-1 levels were elevated in patients with metastases to regional lymph nodes (P<0.001), only higher VEGF levels were associated with higher biopsy and final Gleason sum (P=0.036 and P=0.040, respectively) and extraprostatic extension (P=0.047). Higher pre-operative VEGF level was associated with lymph node involvement and biochemical progression (P=0.043 and P=0.020, respectively), when adjusted for the effects of standard pre-operative features. Thus, plasma VEGF and sVCAM-I levels are markedly elevated in men with metastatic prostate cancer. Furthermore, both are independent predictors of biochemical progression after radical prostatectomy, presumably due to an association with microscopic metastatic disease already present at the time of surgery.

The invention thus provides a method to determine the risk of progression of a patient after therapy for prostate cancer. The method comprises contacting a physiological fluid sample, e.g., blood serum or plasma, from a patient before therapy for prostate cancer with an agent that binds to VEGF and/or sVCAM-1 so as to form a complex. Then the amount or level of complex formation is correlated with the risk of progression.

As also described herein, plasma levels of UPA, UPAR, and PAI-1 were measured pre-operatively in 120 consecutive patients who underwent radical prostatectomy for clinically localized disease and post-operatively in 51 of these patients. UPA and UPAR levels but not PAI-1 levels were elevated in prostate cancer patients compared with healthy subjects (P=0.006 and P=0.021, respectively) and were highest in patients with bone metastases. Elevated UPA and UPAR levels were associated with extraprostatic disease (P=0.046 and P=0.050, respectively) and seminal involvement (P=0.041 and P=0.048, respectively). Elevated UPA and UPAR levels were correlated with prostatic tumor volume (P=0.036 and P=0.030, respectively). In multivariate analysis, pre-operative plasma UPA and UPAR levels, as well as biopsy Gleason sum, were independent predictors of prostate cancer progression (P=0.034, P=0.040, and P=0.048, respectively). In patients with disease progression, pre-operative plasma UPA and UPAR levels were higher in patients with features of aggressive disease than in patients with features of non-aggressive disease (P=0.050 and P=0.031, respectively). Thus, in combination with other clinical and pathologic parameters, plasma UPA and UPAR levels may be useful in selecting patients to enroll in clinical neo-adjuvant and adjuvant therapy trials.

Hence, the invention provides a method to determine the risk of progression of a patient after therapy for prostate cancer. The method comprises contacting a physiological fluid sample such as a blood sample, e.g., a serum or plasma sample, obtained from a patient before therapy for prostate cancer, e.g., before a radical prostatectomy for clinically localized prostate cancer, with an agent that binds to UPAR or UPA so as to form a complex. Then the amount or level of complex formation is correlated with the risk of progression.

The invention also provides an apparatus, comprising: a data input means, for input of test information comprising the level or amount of at least one protein in a sample obtained from a mammal, wherein the protein includes, but is not limited to, TGF-β₁, IGF BP-2, IL-6, IL6sR, IGF BP-3, UPA, UPAR, PSA, VEGF and/or sVCAM; a processor, executing a software for analysis of the level or amount of the at least one protein in the sample; wherein the software analyzes the level or amount of the at least one protein in the sample and provides the risk of progression, non-prostate confined disease, extracapsular extent of disease, seminal vesicle involvement, and/or lymph node involvement in the mammal.

As further described herein, 178 patients with no prior history of prostate biopsy, who had prostate cancer diagnosed during an initial systematic 12-core (S12C) biopsy, and who subsequently underwent radical prostatectomy were studied. The comparison groups included the subset of the six standard sextant cores (S6C), the set of six laterally directed cores (L6C), and the complete 12 core set (S12C) that included both the six standard sextant and six laterally directed cores. Biopsy Gleason score, number of positive cores, total length of cancer, and percent of tumor in the biopsy sets were examined for their ability to predict extracapsular extension, total tumor volume, and pathologic Gleason score. Analyses were performed using Spearman's rho correlation and multivariable regression analyses. In univariable analyses, the S12C correlated most strongly with the presence of extracapsular extension and total tumor volume, compared to either the S6C or the L6C. In multivariable analyses, both the S6C and L6C were independent predictors of post-prostatectomy pathologic parameters. Thus, the addition of 6 systematically obtained, laterally directed cores to the standard sextant biopsy significantly improves the ability to predict pathologic features by a statistically and prognostically or significant margin. Pre-operative nomograms that utilize data from a full complement of 12 systematic sextant and laterally directed biopsy cores can thus improve performance in predicting post-prostatectomy pathology (e.g., indolent cancer or the presence of extracapsular extension). In one embodiment, Gleason score, number of positive cores, number of positive contiguous cores, total cancer length, total length of cancer in contiguous cores, and/or percent tumor involvement are correlated to post-prostatectomy pathology. Moreover, in patients with a negative S12C, initial digital rectal exam status and/or the presence of prostatic intraepithelial neoplasia was found to an indication to rebiopsy, e.g., to perform a second S12C.

To better counsel men diagnosed with prostate cancer, a statistical model that accurately predicts the presence and extent of cancer based on clinical variables (serum PSA, clinical stage, prostate biopsy Gleason grade, and ultrasound volume), and variables derived from the analysis of systematic biopsies, was developed. The analysis included 1,022 patients diagnosed through systematic needle biopsy with clinical stages T1c to T3 NO or NX, and MO or MX prostate cancer who were treated solely with radical prostatectomy. Overall, 105 (10%) of the patients had indolent cancer. The nomogram predicted the presence of an indolent cancer with discrimination for various models ranging from 0.82 to 0.90. Thus, nomograms incorporating pre-treatment variables (clinical stage, Gleason grade, PSA, and/or the amount of cancer in a systematic biopsy specimen) can predict the probability that a man with prostate cancer has an indolent tumor.

The invention provides a method to determine the risk of indolent cancer, or the risk of posterolateral extracapsular extension of prostate cancer, in a patient prior to therapy for prostate cancer. The method comprises correlating one or more of pre-treatment PSA, TGF-β₁, GF BP-2, IL-6, IL6sR, IGF BP-3, UPA, UPAR, VEGF and/or sVCAM; clinical stage; biopsy Gleason scores, number of positive cores, total length of cancer, and/or the percent of tumor in a 12 core set of prostate biopsies from the patient, with the risk of indolent cancer and/or posterolateral extracapsular extension. Such information can enhance treatment decisions.

Hence, the invention also provides a method to predict the presence of indolent prostate tumors. In one embodiment, the method includes correlating a set of factors for a radical prostatectomy patient to a functional representation of a set of factors determined for each of a plurality of patients previously diagnosed with prostate cancer and having been treated by radical prostatectomy, e.g., pre-treatment PSA level, clinical stage, Gleason grade, size of cancerous tissue, size of non-cancerous tissue, and/or ultrasound or transrectal ultrasound (U/S) volume. Then the value for each factor for the patient is correlated to a value on a predictor scale to predict the presence of indolent prostate tumors in the patient.

To develop a nomogram to predict the side of extracapsular extention, 763 patients with clinical stage T1c-T3 prostate cancer who were diagnosed with a systematic biopsy and were subsequently treated with radical prostatectomy were studied. The variables studied included an abnormality on DRE, the worst Gleason score, number of cores with cancer, percent cancer in a biopsy specimen on each side, and serum PSA level. The area under the curve of DRE, biopsy Gleason sum and PSA in predicting the side of ECE was 0.648 and 0.627, respectively, and was 0.763 when these parameters were combined. Further, this was enhanced by adding the information of systematic biopsy with the highest value of 0.787 with a percent cancer. A nomogram incorporating pre-treatment variables on each side of the prostate can thus provide accurate prediction of the side of extracapsular extention in prostate biopsy specimens.

The invention provides a method to predict the side of extracapsular extension in radical prostatectomy specimens. In one embodiment, the method includes correlating a set of factors for a radical prostatectomy patient to a functional representation of a set of factors determined for each of a plurality of patients previously diagnosed with prostate cancer and having been treated by radical prostatectomy, e.g., factors including pre-treatment PSA and, in a biopsy, worst Gleason score, number of cores with cancer, and/or percent cancer in a biopsy specimen on each side. Then the value for each factor for the patient is correlated to a value on a predictor scale to predict the side of extracapsular extension in the prostate of a patient.

To develop a nomogram to improve the accuracy of predicting the freedom from PSA progression after salvage radiotherapy (XRT) for biochemical recurrence following prostatectomy, pre- and post-prostatectomy clinical-pathological data and disease follow-up for 303 patients receiving salvage XRT was modeled using Cox proportional hazards regression analysis. It was found that pre-XRT PSA and Gleason grade were the strongest predictors of treatment success. Thus, a minority of patients may derive a durable benefit from salvage radiotherapy for suspected local recurrence. Accordingly, a nomogram can aid in identifying the most appropriate patients to receive salvage XRT.

Hence, also provided is a method to predict the outcome of salvage radiotherapy after biochemical recurrence in prostate cancer patients treated with radical prostatectomy. In one embodiment, the method includes correlating a set of factors for a radical prostatectomy patient to a functional representation of a set of factors determined for each of a plurality of patients previously diagnosed with prostate cancer and having been treated by radical prostatectomy, e.g., pre-treatment PSA level, pre-salvage radiotherapy PSA level, Gleason sum, pathological stage, pre-salvage radiotherapy PSA doubling time, positive surgical margins, time to biochemical recurrence, and pre-salvage radiotherapy neoadjuvant hormone therapy. Then the value for each factor for the patient is correlated to a value on a predictor scale to predict the outcome of salvage radiotherapy after biochemical recurrence in prostate cancer patients treated with radical prostatectomy.

The invention also includes the use of nomograms to predict time to death in patients with advanced prostate cancer. In one embodiment, the nomogram predicts time to death in patients with hormone sensitive metastatic prostate cancer. In another embodiment, the nomogram predicts the time to death in patients with hormone refractory prostate cancer. Nomograms may include markers present in physiological fluids, e.g., TGF-β₁, UPA, VEGF, and the like, as well as standard clinical parameters, including those in Smaletz et al. (2002), the disclosure of which is specifically incorporated by reference herein. Moreover, the presence of certain markers after primary therapy, e.g., PSA recurrence after primary therapy, may be employed to predict the aggressiveness of recurrence, the time to metastases, and/or time to death.

To determine whether transition zone volume (TZV) and total prostate volume (TPV) are independent predictors of PSA, results from 560 men who underwent a systematic 12-core biopsy performed under ultrasound guidance were analyzed. When controlling for race, age and biopsy status using linear regression, TZV and TPV are each separately significant predictors of PSA (P<0.0001 each).

### BRIEF DESCRIPTION OF THE FIGURES

FIG. 1. Kaplan-Meier estimates of PSA progression-free probability for the 120 patients with clinically localized prostate cancer treated with radical prostatectomy stratified into groups above or below the median TGF-β₁ level of 4.9 ng/mL.

FIG. 2. Box plot of the distribution analysis for TGF-β₁ levels stratified by progression status at 48 months in healthy men without cancer (n 44), consecutive radical prostatectomy patients according to pathologic stage (OC=Organ confined; ECE=Extracapsular extension; SVI=Seminal vesicle involvement; LN Mets=Lymph node metastases) with at least 48 months of follow-up (n=109), men with prostate cancer metastatic to regional lymph nodes (LN Mets, n=19), and men with prostate cancer metastatic to bone (Bone Mets, n=10). Data are presented as median, interquartile and overall range.

FIG. 3. Kaplan-Meier estimates of PSA progression-free probability for the 120 patients with clinically localized prostate cancer treated with radical prostatectomy stratified into groups above or below the median IGF BP-3 level of 3239.8 ng/mL.

FIG. 4. Kaplan-Meier estimates of PSA progression-free probability for the 120 patients with clinically localized prostate cancer treated with radical prostatectomy stratified into groups above or below the median IGF BP-2 level of 437.4 ng/mL.

FIG. 5. Pre-operative and post-operative values for IGF-1, IGF BP-2 and IGF BP-3.

FIG. 6. (A) Kaplan-Meier estimates of PSA progression-free probability for the 120 patients with clinically localized prostate cancer treated with radical prostatectomy stratified into groups above or below the median IL-6 level of 1.9 ng/mL. (B) Kaplan-Meier estimates of PSA progression-free probability for the 120 patients with clinically localized prostate cancer treated with radical prostatectomy stratified into groups above or below the median IL-6 level of 1.9 pg/mL.

FIG. 7. Kaplan-Meier estimates of PSA progression-free probability for the 120 patients with clinically localized prostate cancer treated with radical prostatectomy stratified into groups above or below the median IL6sR level of 25.4 ng/mL.

FIG. 8. Box plot of the distribution analysis for IL-6 levels stratified by progression status at 48 months in healthy men without cancer (n=44), consecutive radical prostatectomy patients according to pathologic stage with at least 48 months of follow-up (n=109), men with prostate cancer metastatic to regional lymph nodes (n=19), and men with prostate cancer metastatic to bone (n=10). Data are presented as median, interquartile and overall range.

FIG. 9. Box plot of the distribution analysis for IL6sR levels stratified by progression status at 48 months in healthy men without cancer (n=44), consecutive radical prostatectomy patients according to pathologic stage with at least 48 months of follow-up (n=109), men with prostate cancer metastatic to regional lymph nodes (n=19), and men with prostate cancer metastatic to bone (n=10). Data are presented as median, interquartile and overall range.

FIG. 10. Survival analysis according to the median TGF- (DMOS follow-up time since surgery).

FIG. 11. Survival analysis according to the median IL6sR (DMOS=follow-up time since surgery).

FIG. 12. Pre-treatment nomogram for predicting recurrence in patients with clinically localized prostate cancer.

FIG. 13. Kaplan-Meier estimates of disease-free probability with 95% confidence bands for 713 patients with clinically localized (T1-3a, NX MO) prostate cancer treated with radical prostatectomy. Numbers above the months indicate patients at risk for recurrence.

FIG. 14. Calibration of the nomogram. Dashed line is reference line where an ideal nomogram would lie. Solid line is performance of current nomogram. Circles are subcohorts of the dataset. X is bootstrap corrected estimate of nomogram performance. Vertical bars are 95% confidence intervals.

FIG. 15. Distribution of nomogram predictions within classic “low” and “high” risk groups. Patients are first classified by risk group as defined by D'Amico et al. Within each risk group is a histogram of the predicted probabilities from the nomogram.

FIGS. 16A-C. Nomograms which include a post-operative blood marker, i.e., TGF-β₁. ECELEV=level of extracapsular extension. 0=not reaching capsule; 1=abutting but not invading capsule; 2=invading but not through capsule; 4=focal extracapsular extension; 5=extensive extracapsular extension.

FIG. 17. Diagram of posterior view of prostate with systematic 12-core biopsy locations marked. Coronal view. Inner circle represents prostatic transition zone. Inner ellipsoid represents transitional zone. X, sextant locations; O, laterally directed locations; ML, midline; B, base; M, mid; A, apex. The circle indicates the anterioposterior and lateral extant of the translational zone in a patient with moderate BPH.

FIG. 18. Nomogram to predict the side of extracapsular extension in radical prostatectomy specimens. BXTGS=biopsy total Gleason score; CSTAGE=clinical stage; PERCA=percent cancer in a biopsy specimen.

FIG. 19. Nomogram to predict progression-free probability post-radiotherapy.

FIG. 20. Nomogram to predict the presence of indolent prostate tumors.

FIG. 21. Plasma UPA and UPAR levels in various patient populations.

FIG. 22. Flow chart.

FIG. 23. Nomogram for patients with hormone refractory disease.

### DETAILED DESCRIPTION OF THE INVENTION

The invention includes a method to predict organ confined (local) prostate disease status, the potential for progression of prostate cancer following primary therapy, e.g., the presence of occult metastases, the side and extent of extracapsular extension of prostate cancer, the risk of extracapsular extension in the area of the neurovascular bundle (posterolaterally), and/or the presence of indolent prostate tumor in patients; the aggressiveness of disease, time to metastasis and/or time to death in patients with PSA recurrence; and the aggressiveness of disease and/or time to death in patients with metastases, e.g., those with or without hormone refractory disease. In one embodiment, the method is particularly useful for evaluating patients at risk for recurrence of prostate cancer following primary therapy for prostate cancer. Specifically, the detection of pre- or post-operative TGF-β₁, IL-6, IL6sR, IGF BP-2, IGF BP-3, UPA, UPAR, VEGF, sVCAM or PSA levels alone, or in conjunction with parameters derived from a 12-core systemic biopsy of the prostate, final pathology, or yet other markers for prostate cancer, may be useful in predicting, for example, organ-confined disease status or the potential for progression in patients with clinically localized prostate cancer.

Non-invasive prognostic assays are provided by the invention to detect and/or quantitate TGF-β₁, IL-6, IL6sR, IGF BP-2, IGF BP-3 UPA, UPAR, VEGF, sVCAM, or PSA levels in the body fluids of mammals, including humans. Thus, such an assay is useful in prognosis of prostate cancer. Moreover, such assays provide valuable means of monitoring the status of the prostate cancer. In addition to improving prognostication, knowledge of the disease status allows the attending physician to select the most appropriate therapy for the individual patient. For example, patients with a high likelihood of relapse can be treated rigorously. Because of the severe patient distress caused by the more aggressive therapy regimens as well as prostatectomy, it would be desirable to distinguish with a high degree of certainty those patients requiring aggressive therapies as well as those which will benefit from prostatectomy.

The body fluids that are of particular interest as physiological samples in assaying for TGF-β₁, IL-6, IL6sR, IGF BP-2, IGF BP-3, UPA, UPAR, VEGF, sVCAM or PSA according to the methods of this invention include blood, blood serum, semen, saliva, sputum, urine, blood plasma, pleural effusions, bladder washes, bronchioalveolar lavages, and cerebrospinal fluid. Blood, serum and plasma are preferred, and plasma, such as platelet-poor plasma, are the more preferred samples for use in the methods of this invention.

Exemplary means for detecting and/or quantitating TGF-β₁, IL-6, IL6sR, IGF BP-2, IGF BP-3, UPA, UPAR, VEGF, sVCAM or PSA levels in mammalian body fluids include affinity chromatography, Western blot analysis, immunoprecipitation analysis, and immunoassays, including ELISAs (enzyme-linked immunosorbent assays), RIA (radioimmunoassay), competitive EIA or dual antibody sandwich assays. In such immunoassays, the interpretation of the results is based on the assumption that the TGF-β₁, IL-6, IL6sR, IGF BP-2, IGF BP-3, UPA, UPAR, VEGF, sVCAM or PSA binding agent, e.g., a TGF-β₁, IL-6, IL6sR, IGF BP-2, IGF BP-3, UPA, UPAR, VEGF, sVCAM, or PSA specific antibody, will not cross-react with other proteins and protein fragments present in the sample that are unrelated to TGF-β₁, IL-6, IL6sR, IGF BP-2, IGF BP-3, UPA, UPAR, VEGF, sVCAM, or PSA. Preferably, the method used to detect TGF-β₁, IL-6, IL6sR, IGF BP-2, IGF BP-3, UPA, UPAR, VEGF, sVCAM or PSA levels employs at least one TGF-β₁, IL-6, IL6sR, IGF BP-2, IGF BP-3, UPA, UPAR, VEGF, sVCAM or PSA specific binding molecule, e.g., an antibody or at least a portion of the ligand for any of those molecules. Immunoassays are a preferred means to detect TGF-β₁, IL-6, IL6sR, IGF BP-2, IGF BP-3, UPA, UPAR, VEGF, sVCAM or PSA. Representative immunoassays involve the use of at least one monoclonal or polyclonal antibody to detect and/or quantitate TGF-β₁, IL-6, IL6sR, IGF BP-2, IGF BP-3, UPA, UPAR, VEGF, sVCAM or PSA in the body fluids of mammals. The antibodies or other binding molecules employed in the assays may be labeled or unlabeled. Unlabeled antibodies may be employed in agglutination; labeled antibodies or other binding molecules may be employed in a wide variety of assays, employing a wide variety of labels.

Suitable detection means include the use of labels such as radionucleotides, enzymes, fluorescers, chemiluminescers, enzyme substrates or co-factors, enzyme inhibitors, particles, dyes and the like. Such labeled reagents may be used in a variety of well known assays. See for example, U.S. Pat. Nos. 3,766,162, 3,791,932, 3,817,837, and 4,233,402.

Still further, in, for example, a competitive assay format, labeled TGF-β₁, IL-6, IL6sR, IGF BP-2, IGF BP-3, UPA, UPAR, VEGF, sVCAM or PSA peptides and/or polypeptides can be used to detect and/or quantitate TGF-β₁, IL-6, IL6sR, IGF BP-2, IGF BP-3, UPA, UPAR, VEGF, sVCAM or PSA, respectively, in mammalian body fluids. Also, alternatively, as a replacement for the labeled peptides and/or polypeptides in such a representative competitive assay, labeled anti-idiotype antibodies that have been prepared against antibodies reactive with TGF-β₁, IL-6, IL6sR, IGF BP-2, IGF BP-3, UPA, UPAR, VEGF, sVCAM or PSA can be used.

It can be appreciated that certain molecules such as TGF-β₁ may be present in various forms, e.g., latent and active, as well as fragments thereof, and that these various forms may be detected and/or quantitated by the methods of the invention if they contain one or more epitopes recognized by the respective binding agents. For example, in a sandwich assay where two antibodies are used as a capture and a detection antibody, respectively, if both epitopes recognized by those antibodies are present on at least one form of, for example, TGF-β₁, the form would be detected and/or quantitated according to such an immunoassay. Such forms which are detected and/or quantitated according to methods of this invention are indicative of the presence of the active form in the sample.

For example, TGF-β₁, IL-6, IL6sR, IGF BP-2, IGF BP-3, UPA, UPAR, VEGF, sVCAM or PSA levels may be detected by an immunoassay such as a “sandwich” enzyme-linked immunoassay (see Dasch et al., 1990; Danielpour et al., 1989; Danielpour et al., 1990; Lucas et al., 1990; Thompson et al., 1989; and Flanders et al., 1989). A physiological fluid sample is contacted with at least one antibody specific for TGF-β₁, IL-6, IL6sR, IGF BP-2, IGF BP-3, UPA, UPAR, VEGF, sVCAM or PSA to form a complex with said antibody and TGF-β₁, IL-6, IL6sR, IGF BP-2, IGF BP-3, UPA, UPAR, VEGF, sVCAM or PSA. Then the amount of TGF-β, in the sample is measured by measuring the amount of complex formation. Representative of one type of ELISA test is a format wherein a solid surface, e.g., a microtiter plate, is coated with antibodies to TGF-β₁, IL-6, IL6sR, IGF BP-2, IGF BP-3, UPA, UPAR, VEGF, sVCAM or PSA and a sample of a patient's plasma is added to a well on the microtiter plate. After a period of incubation permitting any antigen to bind to the antibodies, the plate is washed and another set of TGF-β₁, IL-6, IL6sR, IGF BP-2, IGF BP-3, UPA, UPAR, VEGF, sVCAM or PSA antibodies, e.g., antibodies that are linked to a detectable molecule such as an enzyme, is added, incubated to allow a reaction to take place, and the plate is then rewashed. Thereafter, enzyme substrate is added to the microtiter plate and incubated for a period of time to allow the enzyme to catalyze the synthesis of a detectable product, and the product, e.g., the absorbance of the product, is measured.

It is also apparent to one skilled in the art that a combination of antibodies to TGF-β₁, IL-6, IL6sR, IGF BP-2, IGF BP-3, UPA, UPAR, VEGF, sVCAM or PSA can be used to detect and/or quantitate the presence of TGF-β₁, IL-6, IL6sR, IGF BP-2, IGF BP-3, UPA, UPAR, VEGF, sVCAM or PSA in the body fluids of patients. In one such embodiment, a competition immunoassay is used, wherein TGF-β₁, IL-6, IL6sR, IGF BP-2, IGF BP-3, UPA, UPAR, VEGF, sVCAM or PSA is labeled, and a body fluid is added to compete the binding of the labeled TGF-β₁, IL-6, IL6sR, IGF BP-2, IGF BP-3, UPA, UPAR, VEGF, sVCAM or PSA to antibodies specific for TGF-β₁, IL-6, IL6sR, IGF BP-2, IGF BP-3, UPA, UPAR, VEGF, sVCAM or PSA. Such an assay could be used to detect and/or quantitate TGF-β₁, IL-6, IL6sR, IGF BP-2, IGF BP-3, UPA, UPAR, VEGF, sVCAM or PSA.

Thus, once binding agents having suitable specificity have been prepared or are otherwise available, a wide variety of assay methods are available for determining the formation of specific complexes. Numerous competitive and non-competitive protein binding assays have been described in the scientific and patent literature and a large number of such assays are commercially available. Exemplary immunoassays which are suitable for detecting a serum antigen include those described in U.S. Pat. Nos. 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Methods to detect TGF-β₁ levels as well as TGF-β₁ binding molecules are well known to the art (see, e.g., U.S. Pat. Nos. 5,216,126, 5,229,495, 5,571,714, and 5,578,703; WO 91/08291; WO 93/09228; WO 93/09800; and WO 96/36349).

The methods of the invention may be employed with other measures of prostate cancer biology to better predict disease-free status or for staging. For example, the following clinical and pathological staging criteria may be used, e.g., clinical or pathological stage, PSA levels, Gleason values, e.g., primary Gleason grade, secondary Gleason grade, or Gleason sum (score) and/or core data, although the use of other criteria does not depart from the scope and spirit of the invention.

T0—No evidence of prostatic tumor.

T1—Clinically inapparent tumor, non-palpable nor visible by imaging.

T1a—Tumor is incidental histologic finding with three of fewer microscopic foci. Non-palpable, with 5% or less of TURP chips (trans-urethral resected prostate tissue) positive for cancer.

T1b—Tumor is incidental histologic finding with more than three microscopic foci. Non-palpable, with greater than 5% of TURP chips (trans-urethral resected prostate tissue) positive for cancer.

T1c—Tumor is non-palpable, and is found in one or both lobes by needle biopsy diagnosis.

T2—Tumor is confined within the prostate.

T2a —Tumor present clinically or grossly, limited to the prostate, tumor 1.5 cm or less in greatest dimension, with normal tissue on at least three sides. Palpable, half of 1 lobe or less.

T2b —Tumor present clinically or grossly, limited to the prostate, tumor more than 1.5 cm in greatest dimension, or in only one lobe. Palpable, greater than half of 1 lobe but not both lobes.

T2c—Tumor present clinically or grossly, limited to the prostate, tumor more than 1.5 cm in greatest dimension, and in both lobes. Palpable, involves both lobes.

T3—Tumor extends through the prostatic capsule.

T3a—Palpable tumor extends unilaterally into or beyond the prostatic capsule, but with no seminal vesicle or lymph node involvement. Palpable, unilateral capsular penetration.

T3b—Palpable tumor extends bilaterally into or beyond the prostatic capsule, but with no seminal vesicle or lymph node involvement. Palpable, bilateral capsular penetration.

T3c—Palpable tumor extends unilaterally and/or bilaterally beyond the prostatic capsule, with seminal vesicle and/or lymph node involvement. Palpable, seminal vesicle or lymph node involvement.

T4—Tumor is fixed or invades adjacent structures other than the seminal vesicles or lymph nodes.

T4a—Tumor invades any of: bladder neck, external sphincter, rectum.

T4b—Tumor invades levator muscles and/or is fixed to pelvic wall.

TABLE 1

Gleason grade in biopsy†

| Primary | Secondary | No. patients (%) |
|---|---|---|
| 1-2 | 1-2 | 108 (11.0) |
| 1-2 | 3 | 158 (16.1) |
| 3 | 1-2 | 65 (6.6) |
| 3 | 3 | 340 (34.6) |
| 1-3 | 4-5 | 213 (21.7) |
| 4-5 | 1-5 | 99 (10.1) |

†Gleason grades 1-2 are well differentiated, 3 is moderately differentiated, 4-5 are poorly differentiated.

TABLE 2

| Pre-operative PSA‡ | No. patients (%) |
|---|---|
| 0.1-4.0 | 217 (22.1) |
| 4.1-10.0 | 472 (48.0) |
| 10.1-20.0 | 187 (19.0) |
| 20.1-100.0 | 107 (10.9) |

‡Median serum prostate-specific antigen (PSA) level for all patients. 6.8 ng/mL (range, 0.1-100.0 ng/mL); mean serum PSA level for all patients, 9.9 ng/mL (95% confidence interval = 9.24-10.54 ng/mL).

Exemplary Methods, Apparatus and Nomograms with Pre-Operative Variables

The present invention provides methods, apparatus and nomograms to predict disease recurrence using factors available prior to surgery, to aid patients considering radical prostatectomy to treat clinically localized prostate cancer, as well as to predict disease recurrence after salvage radiation therapy in prostate cancer patients, to predict extracapsular extension in prostate cancer patients, prostatic intraepithelial neoplasia in prostate cancer patients, and/or indolent cancer in prostate cancer patients. In one embodiment, a pre-operative nomogram predicts the probability of disease recurrence after radical prostatectomy for localized prostate cancer (cT1-T3a NO or NX MO or MX) using pre-operative factors, to assist the physician and patient in deciding whether or not radical prostatectomy is an acceptable treatment option. The present invention also provides for post-operative nomograms using selected variables. These nomograms can be used in clinical decision making by the clinician and patient and can be used to identify patients at high risk of disease recurrence who may benefit from neoadjuvant treatment protocols.

Accordingly, one embodiment of the invention is directed to a method for predicting the probability of recurrence of prostate cancer following radical prostatectomy in a patient diagnosed as having prostate cancer. The method comprises correlating a selected set of pre-operative factors determined for each of a plurality of persons previously diagnosed with prostatic cancer and having been treated by radical prostatectomy with the incidence of recurrence of prostatic cancer for each person of the plurality of persons, so as to generate a functional representation of the correlation. The selected set of pre-operative factors includes, but is not limited to, pre-treatment blood TGF-β₁, IL6sR, sVCAM, VEGF, UPAR, UPA, and/or PSA; primary Gleason grade in the biopsy specimen; secondary Gleason grade in the biopsy specimen; Gleason sum; pre-radical prostatectomy therapy (e.g., hormone or radiation); and/or clinical stage; and matching an identical set of pre-operative factors determined from the patient diagnosed as having prostatic cancer to the functional representation so as to predict the probability of recurrence of prostatic cancer, organ confined disease, extracapsular extension, seminal vesical involvement, and lymph node status in the patient following radical prostatectomy. In an alternative embodiment, combined Gleason grade may be used instead of primary and secondary Gleason grades. The combined grade in the biopsy specimen (Bx Gleason Grade) includes the Gleason grade of the most predominant pattern of prostate cancer present in the biopsy specimen (the primary Gleason grade) plus the second most predominant pattern (secondary Gleason grade), if that pattern comprises at least 5% of the estimated area of the cancer or the histologic sections of the biopsy specimen. The terms “correlation,” “correlate” and “correlating” include a statistical association between factors and outcome, and may or may not be equivalent to a calculation of a statistical correlation coefficient.

In one embodiment, the correlating includes accessing a memory storing the selected set of factors. In another embodiment, the correlating includes generating the functional representation and displaying the functional representation on a display. In one embodiment, the displaying includes transmitting the functional representation from a source. In one embodiment, the correlating is executed by a processor or a virtual computer program. In another embodiment, the correlating includes determining the selected set of pre-operative factors. In one embodiment, determining includes accessing a memory storing the set of factors from the patient. In another embodiment, the method further comprises transmitting the quantitative probability of recurrence of prostatic cancer. In yet another embodiment, the method further comprises displaying the functional representation on a display. In yet another embodiment, the method further comprises inputting the identical set of factors for the patient within an input device. In another embodiment, the method further comprises storing any of the set of factors to a memory or to a database.

In one embodiment, the functional representation is a nomogram and the patient is a pre-surgical candidate including patients who have not been previously treated for prostate cancer. In one embodiment, the plurality of persons comprises persons with clinically localized prostate cancer not treated previously by radiotherapy, cryotherapy and/or hormone therapy, who have subsequently undergone radical prostatectomy. In this embodiment, the probability of recurrence of prostatic cancer is a probability of remaining free of prostatic cancer five years following radical prostatectomy. Disease recurrence may be characterized as an increased serum PSA level, preferably greater than or equal to 0.4 ng/mL. Alternatively, disease recurrence may be characterized by positive biopsy, bone scan, or other imaging test or clinical parameter. Recurrence may alternatively be characterized as the need for or the application of further treatment for the cancer because of the high probability of subsequent recurrence of the cancer.

In one embodiment, the nomogram is generated with a Cox proportional hazards regression model (Cox, 1972, the disclosure of which is specifically incorporated by reference herein). This method predicts survival-type outcomes using multiple predictor variables. The Cox proportional hazards regression method estimates the probability of reaching a certain end point, such as disease recurrence, over time. In another embodiment, the nomogram may be generated with a neural network model (Rumelhart et al., 1986, the disclosure of which is specifically incorporated by reference herein). This is a non-linear, feed-forward system of layered neurons which backpropagate prediction errors. In another embodiment, the nomogram may be generated with a recursive partitioning model (Breiman et al., 1984, the disclosure of which is specifically incorporated by reference herein). In yet another embodiment, the nomogram is generated with support vector machine technology (Cristianni et al., 2000; Hastie, 2001). In a further embodiment, e.g., for hormone refractory patients, an accelerated failure time model may be employed (Harrell, 2001). Other models known to those skilled in the art may alternatively be used. In one embodiment, the invention includes the use of software that implements Cox regression models or support vector machines to predict recurrence, disease-specific survival, disease-free survival and/or overall survival.

The nomogram may comprise an apparatus for predicting probability of disease recurrence in a patient with prostatic cancer following a radical prostatectomy. The apparatus comprises a correlation of pre-operative factors determined for each of a plurality of persons previously diagnosed with prostatic cancer and having been treated by radical prostatectomy with the incidence of recurrence of prostatic cancer for each person of the plurality of persons, the pre-operative factors include pre-treatment plasma TGF-β₁, IL6sR, sVCAM, VEGF, PSA, UPAR, UPA, and/or PSA; primary Gleason grade in the biopsy specimen; secondary Gleason grade in the biopsy specimen; and/or clinical stage; and a means for matching an identical set of pre-operative factors determined from the patient diagnosed as having prostatic cancer to the correlation to predict the probability of recurrence of prostatic cancer in the patient following radical prostatectomy.

Another embodiment of the invention is directed to a pre-operative nomogram which incorporates pre-treatment plasma TGF-β₁, IL6sR, sVCAM, PSA, UPAR, UPA, VEGF, and/or PSA; Gleason grade in the biopsy specimen; secondary Gleason grade in the biopsy specimen; and/or clinical stage; as well as one or more of the following additional factors: 1) total length of cancer in the biopsy cores; 2) number of positive cores; and 3) percent of tumor, in a 12 core biopsy set, as well as with other routinely determined clinical factors. For example, and not by way of limitation, if available pre-operatively, one or more of the factors p53, Ki-67, p27 or E-cadherin may be included (Stapleton et al., 1998; Yang et al., 1998).

With respect to the total length of cancer in the biopsy cores, it is customary during biopsy of the prostate to take multiple cores systematically representing each region of the prostate. With respect to the percent of cancerous tissue that percentage is calculated as the total number of millimeters of cancer in the cores divided by the total number of millimeters of tissue collected.

The present invention further comprises a method to predict a pre-operative prognosis in a patient comprising matching a patient-specific set of pre-operative factors such as pre-treatment plasma TGF-β₁, IL6sR, sVCAM, PSA, VEGF, UPA, UPAR, primary Gleason grade in the biopsy specimen, secondary Gleason grade in the biopsy specimen, and/or clinical stage, and determining the pre-operative prognosis of the patient.

The nomogram or functional representation may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The apparatus comprising a nomogram may further comprise a storage mechanism, wherein the storage mechanism stores the nomogram; an input device that inputs the identical set of factors determined from a patient into the apparatus; and a display mechanism, wherein the display mechanism displays the quantitative probability of recurrence of prostatic cancer. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative probability of recurrence of prostatic cancer, e.g., the display is separated from the processor such that the display receives the quantitative probability of recurrence of prostatic cancer. The apparatus may further comprise a database, wherein the database stores the correlation of factors and is accessible by the processor. The apparatus may further comprise an input device that inputs the identical set of factors determined from the patient diagnosed as having prostatic cancer into the apparatus. The input device stores the identical set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the identical set of factors determined from the patient diagnosed as having prostatic cancer, preferably the transmission medium is coupled to the processor and the correlation of factors. The processor may be a multi-purpose or a dedicated processor. The processor includes an object oriented program having libraries, said libraries storing said correlation of factors.

In one embodiment, the nomogram comprises a graphic representation of a probability that a patient with prostate cancer will remain free of disease following radical prostatectomy comprising a substrate or solid support, and a set of indicia on the substrate or solid support, the indicia including one or more of a pre-treatment TGF-β₁ level line, a pre-treatment IL6sR level line, a pre-treatment sVCAM level line, a pre-treatment VEGF level line, a pre-treatment PSA level line, a pre-treatment UPAR level line, a pre-treatment UPA level line, a clinical stage level line, a primary Gleason grade in the biopsy line, and/or a secondary Gleason grade in the biopsy line, a points line, a total points line and a predictor line, wherein the pre-treatment TGF-β₁ level line, pre-treatment IL6sR level line, pre-treatment sVCAM level line, pre-treatment VEGF level line, pre-treatment PSA level line, pre-treatment UPAR level line, pre-treatment UPA level line, clinical stage level line, primary Gleason grade in the biopsy line, and/or a secondary Gleason grade in the biopsy line, each have values on a scale which can be correlated with values on a scale on the points line. The total points line has values on a scale which may be correlated with values on a scale on the predictor line, such that the value of each of the points correlating with the indicia can be added together to yield a total points value, and the total points value correlated with the predictor line to predict the probability of recurrence. The solid support is preferably a laminated card that can be easily carried on a person.

Following radical prostatectomy designed to cure the patient of his cancer, the serum PSA should become undetectable (Stein et al., 1992). Measurable levels of PSA after surgery provide evidence of disease recurrence which may precede detection of local or distant recurrence by many months to years (Partin et al., 1994). Elevated PSA levels are one measure to assess whether radical prostatectomy has cured a patient with prostate cancer, provided that the follow-up is long enough. This association has been demonstrated for patients with a rising PSA after non-hormonal systemic therapy for advanced prostate cancer, for example, in which men with recurrent cancer evidenced by a rising PSA are more likely to die of prostate cancer earlier than men whose PSA does not rise (Sridhara et al., 1995). Serum PSA after radical prostatectomy has been used as an endpoint for treatment efficacy to develop a model which predicts treatment failure. The recurrence decision rule of two PSAs equal to or above 0.03, 0.1 or 0.2 ng/mL and rising can be used as it is relatively safe from indicating false positives, which are particularly undesirable for the patient. Furthermore, using a particular level of PSA as an event indicates that PSA follow-up data are interval-censored (occurring between two time points) (Dorey et al., 1993) rather than right-censored (simply unknown after last follow-up), as modeled. However, adjuvant treatment decisions are often based on observed PSA recurrences, so that this endpoint is more useful clinically than the true PSA recurrence time.

In addition to assisting the patient and physician in selecting an appropriate course of therapy, the nomograms of the present invention are also useful in clinical trials to identify patients appropriate for a trial, to quantify the expected benefit relative to baseline risk, to verify the effectiveness of randomization, to reduce the sample size requirements, and to facilitate comparisons across studies.

Exemplary Methods, Apparatus and Nomograms with Pre- and Post-Operative Variables

In addition to the various embodiments of the pre-operative nomograms and method of using the nomograms discussed above, the present invention is also directed toward post-operative nomograms and methods of utilizing these nomograms to predict probability of disease recurrence following radical prostatectomy. This prognosis may be utilized, among other reasons, to determine the usefulness of adjuvant therapy in a patient following radical prostatectomy.

Accordingly, further embodiments of the present invention include a nomogram which incorporates factors, including post-operative factors, to predict probability of cancer recurrence after radical prostatectomy for clinically localized prostatic cancer. This nomogram predicts probability of disease recurrence using factors for patients who have received radical prostatectomy to treat clinically localized prostate cancer.

One embodiment of the invention is directed to a post-operative method for predicting probability of recurrence of prostate cancer in a patient who has previously undergone a radical prostatectomy comprising: correlating a set of factors determined for each of a plurality of persons previously diagnosed with prostate cancer with the incidence of recurrence of prostatic cancer for each person of the plurality to generate a functional representation of the correlation. In alternative embodiments, one or more subgroups of any one or more of the following factors may be excluded. The set of factors comprises one or more of the following: (1) post-operative TGF-β₁ level; (2) pre-operative PSA level; (3) pre-operative TGF-β₁ level; (4) prostatic capsular invasion level (ECELEV); (5) pathological Gleason score; (6) surgical margin status; (7) seminal vesicle involvement; (8) lymph node status; (9) pre-operative IL6sR level; (10) prior therapy, wherein said plurality of persons comprises men having undergone radical prostatectomy; and matching an identical set of factors determined from the patient to the functional representation to predict the probability of recurrence of prostatic cancer for the patient. In one embodiment, surgical margin status is reported as negative or positive. Alternatively, surgical margin status may be reported as negative, close or positive. In one embodiment, prostatic capsular invasion level is reported as none, invading the capsule, focal or established.

Seminal vesicle involvement or invasion is preferably reported as yes or no. Alternatively, it may be ranked as positive or negative, or absent or present. If present, seminal vesicle involvement can be alternatively classified by level as Types I, II, I+II, or III (Ohori et al., 1993). In yet another embodiment, seminal vesicle invasion, if present, may be alternatively ranked by level as type I, II, or III (Wheeler, 1989; Ohori et al., 1993). Lymph node status is preferably recorded as either positive or negative.

In another embodiment, the selected set of factors may further include one or more of the following: the volume of cancer (total tumor volume), the zone of the prostate where the tumor is found (zone of location of the cancer), level of extraprostatic extension, pre-treatment UPAR level, pre-treatment UPA level, p53, Ki-67, p27, DNA ploidy status, clinical stage, lymphovascular invasion, and other routinely determined pathological factors (Greene et al., 1991; Greene et al., 1962; Ohori et al., 1993; Stapleton et al., 1998; Yang et al., 1999).

Level of extraprostatic extension may be evaluated as negative, level 1, level 2, level 3 focal, or level 3 established (Stamey et al., 1998; Rosen et al., 1992). Alternatively, level of extraprostatic extension may be evaluated as negative, level 1, level 2 or level 3 focal. Alternatively, level of extraprostatic extension may be evaluated as level 0 or 1 (no invasion of the capsule or extension outside of the prostate), level 2 (invasion into but not through the capsule), level 3F (focal microscopic extension through the capsule comprising no more than two high power fields on all histologic sections), or level 3E (established extension through the capsule more extensive than level 3F) (Greene et al., 1991; Greene et al., 1992; Greene et al., 1991; and Ohori et al., 1993).

The probability of recurrence of prostate cancer includes the probability of remaining free of prostatic cancer five years following radical prostatectomy. Recurrence may be characterized as an increased serum PSA level or as positive biopsy, bone scan, or other suitable imaging test or clinical parameter. Alternatively, recurrence may be characterized as a positive biopsy, bone scan or the initiation or application of further treatment for prostate cancer because of the high probability of subsequent recurrence of the cancer.

In one embodiment, the functional representation is a nomogram. The nomogram may be generated with a Cox proportional hazards regression model (Cox, 1972). Alternatively, the nomogram may be generated with a neural network model (Rumelhart et al., 1986). In another embodiment, the nomogram is generated with a recursive partitioning model (Breiman et al., 1984). In yet another embodiment, the nomogram is generated with support vector machine technology (Cristianni et al., 2000). In a further embodiment, e.g., for hormone refractory patients, an accelerated failure time model may be employed (Harrell, 2001). Other models known to those skilled in the art may alternatively be used.

In one embodiment, the invention includes the use of software that implements Cox regression models or support vector machines to predict recurrence, disease-specific survival, disease-free survival and/or overall survival.

In one embodiment of the invention, the invention is directed to a method to predict a post-operative prognosis in a patient following radical prostatectomy, comprising matching a patient-specific set of factors comprising the patient's pre-operative PSA, TGF-β₁, or IL6sR level, post-operative TGF-β₁ level, pathological Gleason score, prostatic capsular invasion level, surgical margin status, presence of seminal vesicle invasion, and lymph node status, and determining the prognosis of the patient.

Still another embodiment of the invention is directed to a method for determining a need for an adjuvant therapy in a patient following radical prostatectomy comprising the steps of determining a set of clinical and pathological factors on the patient, the set of factors comprising the patient's pre-operative PSA, TGF-β₁, or IL6sR level, post-operative TGF-β₁ level, pathological Gleason score, prostatic capsular invasion level, surgical margin status, presence of seminal vesicle invasion, and lymph node status; and matching the set of factors to determine whether the adjuvant therapy is needed in view of the probability of recurrence. The adjuvant therapy may comprise radiotherapy, chemotherapy, hormonal therapy (such as anti-androgen hormonal therapy), cryotherapy, interstitial radioactive seed implantation, external beam irradiation, hyperthermia, gene therapy, cellular therapy, tumor vaccine, or systemically delivered biologic agents or pharmaceuticals.

Another embodiment of the invention is directed to an apparatus for predicting probability of disease recurrence in a patient with prostate cancer following a radical prostatectomy. The apparatus comprises a correlation of clinical and pathological factors determined for each of a plurality of persons previously diagnosed with prostatic cancer and having been treated by radical prostatectomy with incidence of recurrence of prostatic cancer for each person of the plurality of persons. The selected set of factors comprises pre-operative PSA, pre-operative TGF-β₁, pre-operative IL6sR level, post-operative TGF-β₁ level, pathological Gleason score, prostatic capsular invasion level, surgical margin status, presence of seminal vesicle invasion, and lymph node status; and a means for matching an identical set of factors determined from the patient diagnosed as having prostatic cancer to the correlation to predict the probability of recurrence of prostatic cancer in the patient following radical prostatectomy.

Another embodiment of the invention is directed to a nomogram for the graphic representation of a probability that a patient with prostate cancer will remain free of disease following radical prostatectomy comprising a set of indicia on a solid support, the indicia comprising a pre-operative PSA level line, a pre-operative TGF-β₁ level line, a pre-operative IL6sR level line, a post-operative TGF-β₁ level line, pathological Gleason sum line, a prostatic capsular invasion level line, a surgical margin status line, a presence of seminal vesicle invasion line, a lymph node status line, a points line, a total points line and a predictor line, wherein the pre-operative PSA level line, a pre-operative TGF-β₁ level line, a pre-operative IL6sR level line, a post-operative TGF-β₁ level line, pathological Gleason sum line, prostatic capsular invasion level line, surgical margin status line, presence of seminal vesicle invasion line, and lymph node status line each have values on a scale which can be correlated with values on a scale on the points line, and wherein said total points line has values on a scale which may be correlated with values on a scale on the predictor line, such that the value of each of the points correlating with the patient's pre-operative PSA level, specimen Gleason sum, prostatic capsular invasion level, surgical margin status, presence of seminal vesicle invasion, and lymph node status can be added together to yield a total points value, and the total points value can be correlated with the predictor line to predict the probability of recurrence. The solid support may assume any appropriate form such as, for example, a laminated card. Any other suitable representation, picture, depiction or exemplification may be used.

The invention will be further described by the following non-limiting examples.

### EXAMPLE 1

Materials and Methods

Patient Population

Plasma TGF-β₁ levels were assessed in 44 healthy patients without cancer, in 19 men with prostate cancer metastatic to regional lymph nodes, and in 10 patients with bone scan-proven, metastatic prostate cancer. Neither patients with metastatic lymph node disease nor patients with metastatic bone disease were treated with either hormonal or radiation therapy before plasma collection. The healthy non-cancer group was composed of three sets of patients who presented consecutively to the Baylor Prostate Center's weekly prostate cancer screening program. They had no prior history of any cancer or chronic disease, a normal digital rectal examination, and a PSA of less than 2.0 ng/mL, a PSA range that has an estimated probability of prostate cancer detection of less than 1% in the first 4 years after screening (Smith et al., 1996).

One hundred and twenty consecutive patients were also studied who underwent radical prostatectomy for clinically localized prostatic adenocarcinoma (clinical stage T1 to T2) at The Methodist Hospital, Houston, Tex. No patient was treated pre-operatively with either hormonal or radiation therapy, and none had any secondary cancer. The clinical stage was assigned by the operative surgeon according to the 1992 TNM system. The mean patient age in this study was 61.8±7.2 years (median 63.0, range 40 to 76). Serum prostate specific antigen was measured by the Hybritech® Tandem-R assay (Hybritech, Inc., San Diego, Calif.).

TGF-β₁ Measurements

Serum and plasma samples were collected on an ambulatory basis at least 4 weeks after transrectal guided needle biopsy of the prostate, typically performed on the morning of the scheduled day of surgery after a typical pre-operative overnight fast. Blood was collected into Vacutainer® CPT™ 8 mL tubes containing 0.1 mL of 1 M sodium citrate anticoagulant (Becton Dickinson Vacutainer Systems, Franklin Lakes, N.J.) and centrifuged at room temperature for 20 minutes at 1500×g. The top layer corresponding to plasma was decanted using sterile transfer pipettes and immediately frozen and stored at −80° C. in polypropylene cryopreservation vials (Nalgene, Nalge Nunc International, Rochester, N.Y.). Prior to assessment, an additional centrifugation step of the plasma at 10,000×g for 10 minutes at room temperature for complete platelet removal was performed. For quantitative measurements of platelet-poor plasma and serum TGF-β₁ levels, a quantitative sandwich enzyme immunoassay (Quantikine® Human TGF-β₁ Elisa kit, R&D Systems, Minneapolis, Minn.) was used, that is specific for TGF-β₁ and does not cross-react with TGF-β₂ or TGF-β₃. Recombinant TGF-β₁ was used as standard. Every sample was run in duplicate, and the mean was used for data analysis. Differences between the two measurements were minimal, as shown the intra-assay precision coefficient of variation of only 4.73±1.87%.

TGF-β₁ Collection Formats

In a preliminary study, TGF-β₁ levels were assessed from three synchronously drawn blood specimens obtained from 10 of the 44 healthy screening patients. Plasma was separated using Vacutainer® K₃ ethylenediaminetetraacetic acid (EDTA) 5 mL tubes containing 0.057 mL of 15% K₃ EDTA solution, and Vacutainer® CPT™ 8 mL tubes containing sodium citrate (Becton Dickinson Vacutainer Systems, Franklin Lakes, N.J.). Serum was separated using Vacutainer® Brand SST Serum Separator ^{m} tubes (Becton Dickinson Vacutainer Systems, Franklin Lakes, N.J.). Specimens were centrifuged at room temperature for 20 minutes at 1500×g, and plasma or serum decanted and frozen at −80° C. until assessment. Prior to assay, an additional centrifugation step at 10,000×g for 10 minutes at room temperature was performed. The investigators were blinded to the nature of the collection formats. Analysis of variance was used to determine whether the collection format significantly affected measured TGF-β₁ levels.

Pathological Examination

All prostatectomy specimens were examined pathologically by a single pathologist, who was blinded to clinical outcome. Pelvic lymph nodes were removed in a standard fashion at surgery and examined microscopically for the presence of metastatic prostate cancer. The radical prostatectomy specimens were processed by whole-mount technique, and pathological parameters evaluated as described in Wheeler et al. (1994).

Post-Operative Follow-Up

Each patient had a digital rectal examination and serum PSA post-operatively every 3 months for the first year, semiannually from the second through the fifth year, and annually thereafter. A staging evaluation, including bone scan, prostascint, or PSA doubling time calculation was performed in 11 of the 15 patients who had PSA progression prior to the administration of salvage radiation or hormonal therapy. Biochemical progression was defined as a sustained elevation, on 2 or more occasions, of PSA>0.2 ng/mL. The date of progression was assigned to the date of the first value >0.2 ng/mL. Two (1.7%) patients had lymph node positive disease at the time of radical prostatectomy, and surgery was consequently aborted prior to prostate removal. These patients were categorized as failures from the day after surgery. Two (1.7%) patients received adjuvant radiation therapy before biochemical progression because of positive surgical margins. One of them subsequently experienced PSA relapse and was categorized as having progression from the date of the first value >0.2 ng/mL. There were 17 failures among the 120 men. PSA relapse was the sole indication of progression in 14 patients, while 3 had clinical, in addition to biochemical evidence of progression. Post-progression serum PSA doubling time was calculated for patients that had biochemical progression and at least three PSA measurements after the date of progression using the formula: DT log(2)×T/[log(final PSA)−log(initial PSA)], where DT is the serum PSA doubling time, T is the time interval between the initial and final PSA level, final PSA is the pre-radiation PSA level, and initial PSA is the PSA level noted at the time of the post-operative biochemical recurrence. The natural logarithm was used in all logarithmic transformations. Eight (53%) of the patients that progressed were treated with external beam radiation therapy limited to the prostatic fossa at the Methodist Hospital. Radiation was delivered with 15 to 20 MV photons, and the four-fields technique (anteroposterior/posteroanterior and opposing laterals) with customized field sizes was used. Total radiation therapy dose ranged from 60 to 66 Gy, delivered in daily fractions. A complete response to salvage radiation therapy was defined as the achievement and maintenance of an undetectable serum PSA level. Radiation therapy was considered to have failed if the post-radiation serum PSA levels did not fall to, and remain at, an undetectable level.

Statistical Analysis

Analysis of variance was used to assess differences in TGF-β₁ levels. Multiple comparisons were conducted when the overall test was significant (one way ANOVA followed by Fisher's least significant difference). Pre-operative PSA level had a skewed distribution and so was modeled with a log transformation. Clinical stage was evaluated as T1 versus T2 and biopsy Gleason score was evaluated as grade 2 to 6 versus grade 7 to 10. Differences in TGF-β₁ levels between patients who presumably had distant failure and those who presumably had local-only failure were tested by the Mann-Whitney test. Spearman's rank correlation coefficient was used to compare ordinal and continuous variables. Logistic regression was used for multivariate analysis of binary outcome variables. The Kaplan-Meier method was used to calculate survival functions and differences were assessed with the long rank statistic. Multivariate survival analysis was performed with the Cox proportional hazard regression model. Statistical significance in this study was set as P<0.05. All reported P values are two-sided. All analyses were performed with SPSS statistical package (SPSS version 10.0 for Windows).

Results

Impact of Collection Formats on TGF-β₁ Levels

Initially, the effect of the collection format on TGF-β₁ levels was studied. Mean TGF-β₁ levels, measured in Vacutainer CPT citrate plasma, Vacutainer®K₃ EDTA plasma, and Vacutainer®BrandSST™ serum from synchronously drawn blood specimens of 10 consecutive, healthy screening patients were 4.21±1.16 ng/mL, 8.34±2.94 ng/mL, and 23.89±5.35 ng/mL, respectively (Table 3). TGF-β₁ levels measured in serum were 3-times higher than those in measured in citrate platelet-poor plasma and 6-times higher than those measured in EDTA platelet-poor plasma. Although analysis of variance showed TGF-β₁ inter-collection format differences to be statistically significant (P values <0.001), TGF-β₁ levels measured in specimens collected by all three sample formats were found to be highly correlated with each other (P values <0.001). However, levels of TGF-β₁ measured in specimens from the two platelet-poor plasma formats were the most highly correlated (CC=0.987). Platelet-poor plasma from Vacutainer®CPT™ sodium citrate tubes was used for TGF-β₁ measurements in the study described below.

TABLE 3

TGF-β₁ (ng/Ml)

| Collection Format | Mean ± SD* | Range |
|---|---|---|
| Citrate plasma | 4.21 ± 1.16 | 2.46-5.38 |
| EDTA plasma | 8.34 ± 2.94 | 7.41-16.33 |
| Serum | 23.89 ± 5.35 | 17.28-37.01 |
| Collection Formats | P value† | Correlation Coefficient‡ |
| EDTA plasma and citrate | <0.001 | 0.987 |
| plasma | | |
| EDTA plasma and serum | <0.001 | 0.789 |
| Citrate plasma and serum | <0.001 | 0.801 |

*SD = Standard Deviation.
†P-values (two-sided) were calculated based on analysis of variance in a randomized complete block design for the assessment of the difference in TGF-β₁ levels between collection formats.
‡Spearman correlation coefficients were used to assess the relationship between different collection formats.

Clinical and Pathological Characteristics

All patients had clinically localized (T1 or T2) disease, and the mean pre-operative TGF-β₁ and PSA levels were 5.4+2.0 ng/mL (median 4.9, range 1.66 to 15.1) and 9.5±6.3 ng/mL (median 8.2, range 2.1 to 49.0), respectively. Nine (7.5%) patients had PSA levels less than 4 ng/mL; 75 (62.5%) had PSA levels greater than or equal to 4 ng/mL and less than 10 ng/mL; and 36 (30.0%) had PSA levels greater than or equal to 10 ng/mL. Clinical and pathological characteristics are listed in Table 4. On univariate analysis, pre-treatment TGF-β₁ levels correlated with pre-operative PSA levels (P=0.019) and pathological stage (P<0.001) (Table 5).

TABLE 4
Pre-operative Characteristics

| | Patients | | Patients |
|---|---|---|---|
| Clinical stage | N (%) | Biopsy Gleason score | N (%) |
| cT1 a + b | 1 (0.8) | 2-4 | 3 (2.5) |
| cT′ c | 41 (34.2) | 5-6 | 77 (64.2) |
| cT2 a | 46 (38.3) | 7 | 35 (29.2) |
| cT2 b | 16 (13.3) | 8-10 | 5 (4.1) |
| cT2 c | 16 (13.3) | | |

Post-operative Characteristics

| | Patients | Pathologic Gleason | Patients |
|---|---|---|---|
| Pathological features | N (%) | score* | N (%) |
| Organ Confined | 79 (65.8) | 2-4 | 0 (0) |
| ECE only | 33 (27.5) | 5-6 | 59 (50.0) |
| SVI+ | 8 (6.7) | 7 | 56 (47.5) |
| LN+ | 2 (1.7) | 8-10 | 3 (2.5) |
| SM+ | 16 (13.3) | | |

ECE = Extracapsular extension.
SVI+ = Seminal vesicle invasion.
LN+ = Lymph node positive.
SM+ = Positive surgical margins.
*Gleason tumor grade unavailable for two patients, who did not undergo a prostatectomy because of grossly positive pelvic lymph nodes at the time of surgery.

TABLE 5

| Parameter | Correlation Coefficient* | P value† |
|---|---|---|
| Age | 0.21 | 0.823 |
| Pre-operative PSA | 0.214 | 0.019 |
| Biopsy Gleason sum | 0.117 | 0.204 |
| Clinical stage | −0.076 | 0.412 |
| Final pathologic stage | 0.344 | <0.001 |
| Final pathologic Gleason sum | 0.087 | 0.348 |

*Spearman's correlation coefficients were used to assess the relationship between TGF-β₁ levels and clinicopathological parameters.
†P-values (two-sided) of the Spearman correlation were determined by Wilcoxon's rank sum.

Final Pathological Stage and Progression as a Function of TGF-β₁ and Other Parameters

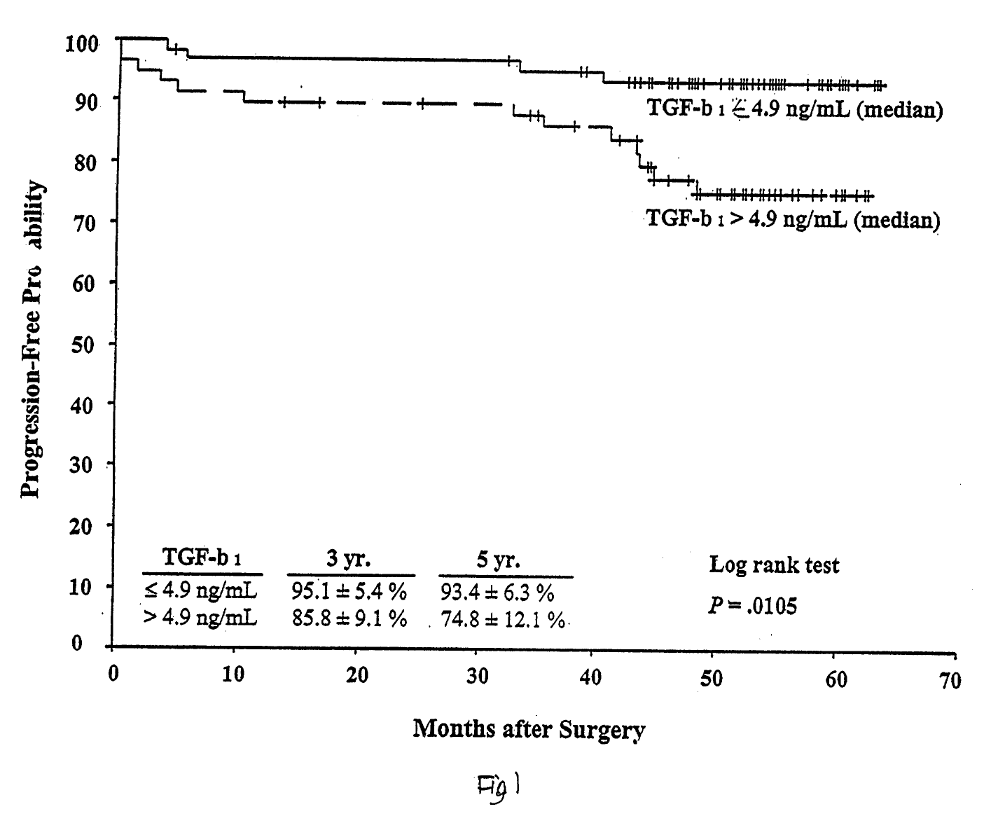

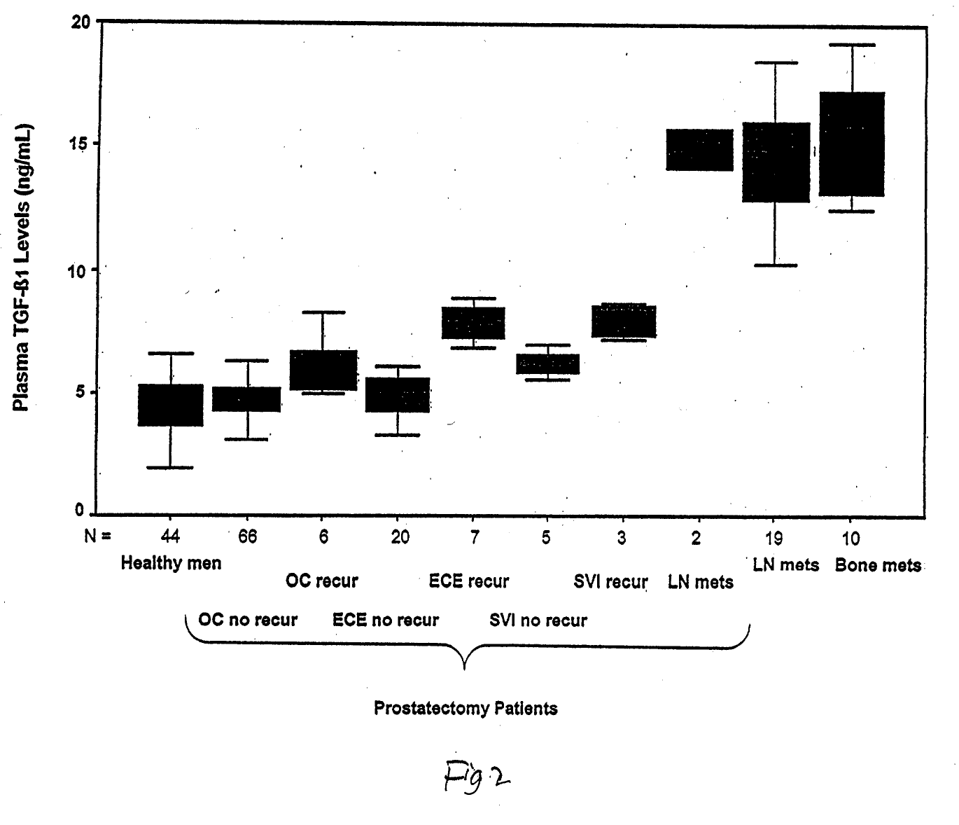

In both an univariate and a multivariate logistic regression analysis that included pre-operative TGF-β₁, pre-operative PSA, clinical stage, and biopsy Gleason score, plasma TGF-β₁ levels (P=0.006; Hazard ratio 0.616, 95% CI 0.436-0.869) and biopsy Gleason grade (P=0.006; Hazard ratio 3.671, 95% CI 1.461-9.219) were significant predictors of organ-confined disease. Overall, only 14% of patients (17 of 120) had cancer progression with a median post-operative follow-up of 53.8 months (range 1.16 to 63.3). The overall PSA progression-free survival was 90.7±5.3% (95% CI) at 3 years and 84.6±6.8% (95% CI) at 5 years. Using the log rank test, it was found that patients with plasma TGF-β₁ levels above the median (4.9 ng/mL) had a significantly increased probability of PSA-progression (P=0.0105; FIG. 1). On univariate Cox proportional hazards regression analysis, plasma TGF-β₁ was associated with the risk of PSA progression (P<0.001) along with biopsy Gleason score (P=0.005, Table 6). In a pre-operative multivariate model that included pre-operative TGF-β₁, pre-operative PSA, clinical stage, and biopsy Gleason score, plasma TGF-β₁ level and Gleason score (P<0.001) were both independent predictors of disease progression.

TABLE 6

| | Univariate | | | Multivariate | | |
|---|---|---|---|---|---|---|
| | Hazard | | | Hazard | | |
| Variable | ratio | P | 95% CI | ratio | P | 95% CI |
| Pre-operative PSA levels* | 5.772 | 0.067 | 0.887-37.547 | 2.408 | 0.363 | 0.362-16.016 |
| Pre-operative TGFβ-₁ levels | 2.246 | <0.001 | 1.637-3.083 | 2.268 | <0.001 | 1.629-3.158 |
| Biopsy Gleason Score† | 4.167 | 0.005 | 1.541-11.273 | 3.582 | 0.021 | 1.212-10.585 |
| Clinical Stage‡ | 1.850 | 0.226 | 0.684-5.002 | 1.646 | 0.351 | 0.578-4.687 |

*Pre-operative PSA levels were logarithmically transformed.
†Biopsy Gleason Score was categorized as grade 2 to 6 versus grade 7 to 10.
‡Clinical stage was categorized as T1 versus T2.

Characteristics of Patients with Disease Progression

Two of the 17 (12%) patients who progressed had lymph node positive disease at the time of radical prostatectomy. Five patients were presumed to have local failure based on PSA doubling times greater than 12 months (n=3; median 19.6, range 15.8-21.6) or complete response to local salvage radiation therapy (n=2). Eight patients were presumed to have distant failure based on metastatic work-up (positive bone scan or prostascint; n=3), PSA doubling times less than 10 months (n=7; median 6.6, range 1.97-9.80), or failure to respond to local salvage radiation therapy (n=1). Pre-operative plasma TGF-β₁ levels were significantly higher in patients with presumed distant failure (median 8, range 6.5-8.9) than those with local failure (median 5.5, range 4.3-8.3; P=0.019).

TGF-β₁ in Healthy and Metastatic Patients

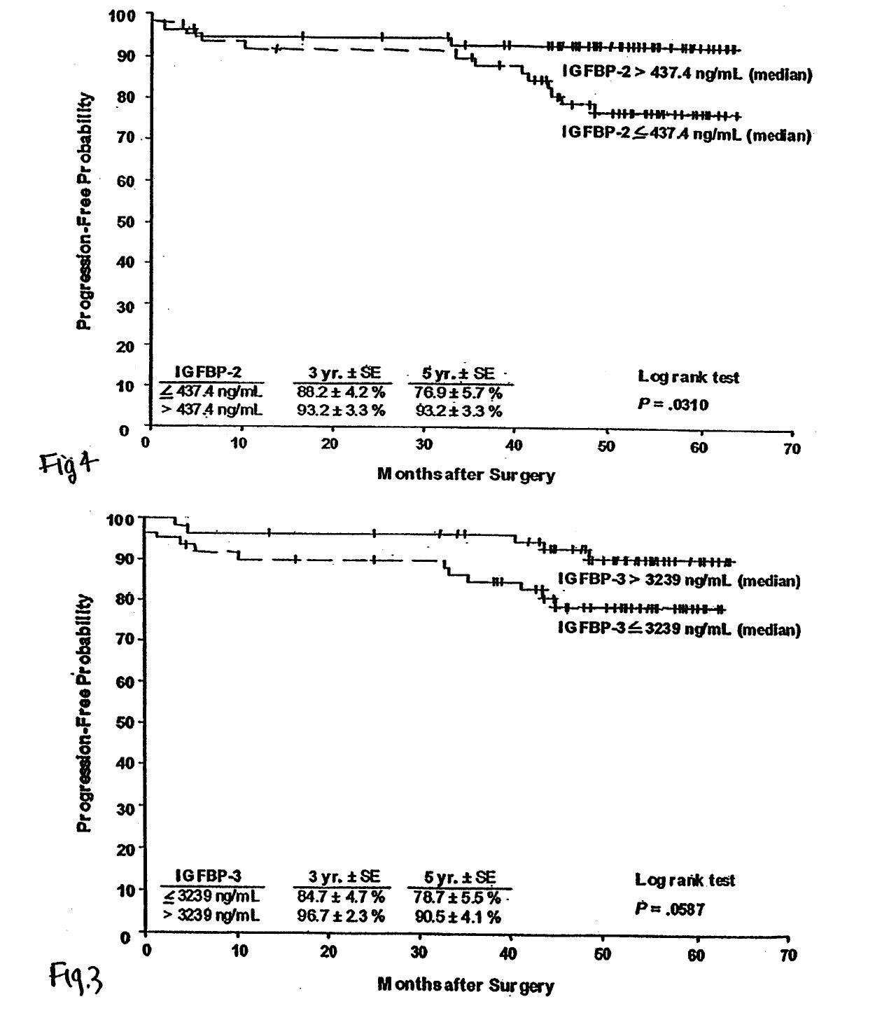

Mean TGF-β₁ levels in the 44 healthy screening patients, the 19 patients with prostate cancer metastatic to regional lymph nodes, and the 10 patients with metastatic prostate cancer were 4.5±1.2 ng/mL (median 4.70, range 1.0-6.6), 14.24±2.6 ng/mL (median 14.95, range 8.0-19.2), and 15.51±2.4 ng/mL (median 15.20, range 12.4-19.3), respectively. Plasma TGF-β₁ levels in patients with lymph node metastases and bone metastases were significantly higher than those in the initial cohort of 120 prostatectomy patients and healthy subjects (P values <0.001). However, plasma TGF-β₁ levels in the initial cohort of 120 prostatectomy patients were not significantly higher than those in healthy subjects (P=0.053). Similarly, plasma TGF-β₁ levels in patients with bone metastases were not significantly different from those in patients with lymph node metastases (P=0.108). TGF-β₁ and Prostate Cancer Stage and Progression FIG. 2 shows box plots of the TGF-β₁ levels in 109 of the 120 consecutive prostatectomy patients who had at least 48 months of follow-up, stratified by progression status at 48 months, 44 healthy men without cancer, 19 men with prostate cancer metastatic to regional lymph nodes, and 10 men with prostate cancer metastatic to bone. TGF-β₁ levels were not different between healthy men, patients with organ confined disease who did not have disease progression, and patients with extracapsular disease who did not have disease progression (P values >0.229). However, TGF-β₁ levels in these three groups were significantly lower than in patients with biochemical progression who had organ confined disease, extracapsular disease, or seminal vesicle invasion, or in patients with lymph node metastases, or patients with bone metastases (P values <0.005). The group of patients with lymph node metastases or bone metastases had similar TGF-β₁ levels (P=0.271), which were significantly higher than those in any of the other groups (P values <0.001).

Discussion

It was confirmed that TGF-β₁ levels are greatly elevated in patients with regional and distant metastases compared to patients with non-metastatic prostate cancer or in healthy subjects. A significant association was found between pre-operative platelet-poor plasma TGF-β₁ levels and established markers of biologically aggressive prostate cancer, such as pre-operative serum PSA levels and final pathologic stage, in a large cohort of consecutive patients with long term follow-up after radical prostatectomy. Furthermore, pre-operative plasma TGF-β₁ was found to be a powerful independent predictor of final pathologic stage and disease progression in patients with clinically localized prostate cancer. Within each pathological stage, patients who developed disease progression had significantly higher TGF-β₁ levels than their non-progressing counterparts. Furthermore, in patients that progressed, pre-operative plasma TGF-β₁ levels were significantly higher in patients with presumed distant failure than those with presumed local-only failure.

In radical prostatectomy patients, the plasma TGF-β₁ level was strongly associated with PSA and pathological stage, two established markers of biologically aggressive prostate cancer. However, in a pre-operative model, TGF-β₁ and biopsy tumor grade but not PSA were independently predictors of advanced pathological stage. An association between elevated TGF-β₁ levels and locally advanced prostate cancer has been previously reported (Ivanovic et al., 1995). In a small pilot study, Ivanovic et al. found that patients with advanced pathological stage had a 2-fold and 4-fold increase in TGF-β₁ levels over patients with confined disease and healthy controls, respectively. However, the majority of patients with organ confined, extracapsular disease, and even seminal vesicle invasion, whose local tumor is completely removed, as evidenced by a negative surgical margin, have long term freedom from biochemical progression (Maru et al., 1999; Epstein et al., 1998; Tefilli et al., 1998; Epstein et al., 2000). On the other hand, most, if not all patients, with lymph node involvement eventually fail local therapy by developing distant metastases, regardless of the success of eradicating local disease (Eastham et al., 2000; Catalona et al., 1998; Walsh et al., 1994). Nomograms consisting of biomarkers that can predict disease progression rather than final pathologic features in patients undergoing radical prostatectomy for prostate cancer would provide greater clinical impact in managing patients with prostate cancer.

A strong association was found between circulating TGF-β, levels and disease progression after radical prostatectomy. To process the radical prostatectomy specimens, a whole-mount step-section technique was used that has been shown to be the most accurate means of detecting positive surgical margins and in determining pathologic stage (Wheeler, 1989). In the present study, the positive margin rate was 13.3%, compared with the 16% to 46% positive margin rates reported by others in patients with clinically localized prostate cancer (Ohori et al., 1995; Jones, 1990). Positive surgical margins may suggest the presence of residual local tumor in the surgical bed which has been shown to be a strong predictor of local recurrence (Epstein, et al., 1996). The lower rate of positive surgical margins (13.3%) and the high rate of presumed distant failures (67%) based on PSA doubling times less than 10 months (Pound et al., 1999), the failure to respond to local salvage radiation therapy or a positive metastatic work up, suggested that the association between pre-operative TGF-β₁ levels and disease progression in these patients was more likely to due to an association with the presence of occult metastatic disease present at the time of surgery, rather than with incomplete resection of potentially curable disease. The finding that patients who failed with presumably distant disease had significantly higher TGF-β₁ levels than those who failed locally supports the hypothesis that TGF-β₁ is associated with occult metastases at time of surgery. To further explore this hypothesis, TGF-β₁ levels were analyzed in 109 of the 120 consecutive prostatectomy patients who had at least 48 months of follow-up, stratified by progression status by 48 months and it was found that pre-operative TGF-β₁ levels were significantly elevated in patients with biochemical progression irrespective of the pathologic stage. Thus, TGF-β₁ could be included in pre-operative nomograms for prediction of progression (Kattan et al., 1998).

To further evaluate the association between TGF-β, and metastases, TGF-β₁ levels were assessed in ten patients with bone-scan proven metastatic disease, in 19 men with prostate cancer metastatic to regional lymph nodes, and 44 healthy men without any cancer. In agreement with all, except one, previous reports, dramatically elevated levels of TGF-β₁ were found in patients with distant prostate cancer metastases (Ivanovic et al., 1995; Adler et al., 1999; Kakehi et al., 1996). The only study that did not detect any association between TGF-β₁ levels and metastases relied on serum samples, which can lead to aberrant TGF-β₁ levels (Wolff et al., 1999). Furthermore, Wolff et al. (1999) did not specify whether any of the metastatic patients were undergoing androgen-deprivation therapy. The present study evaluated patients with metastatic prostate cancer prior to any therapy, including hormonal therapy. To date, only one other group investigated the levels of TGF-β₁ in patients with regional nodal metastases. In agreement with the present findings, Kakehi et al. (1996) detected significantly elevated TGF-β₁ levels in patients with prostate cancer metastatic to regional lymph nodes. However, in contrast to previous studies (Ivanovic et al., 1995; Adler et al., 1999; Kakehi et al., 1996), no overlap was found between TGF-β₁ levels of regional or distant metastatic patients and those from controls or patients with either localized or advanced prostate cancer. The complete separation of TGF-β₁ levels between patients with clinical or pathological evidence of metastatic disease supports the potential use of plasma TGF-β₁ as a staging marker for prostate cancer that could provide clinically meaningful pathological stratification of the patients. Conversely, in concordance with previous studies, no statistically significant difference was found in plasma TGF-β₁ levels between patients with pathologically localized prostate cancer and healthy men without cancer, limiting the value of TGF-β₁ as a diagnostic tool for early detection of localized prostate cancer (Kakehi et al., 1996; Wolff et al., 1999; Perry et al., 1997).

TGF-β₁ levels were found to be 3 to 6-times higher when measured in serum as compared to platelet-poor plasma. Since TGF-β₁ is present in platelet granules and is released upon platelet activation, the highly elevated levels of TGF-β₁ in serum are likely to derive from damaged platelets and not from the prostate, making quantification of TGF-β₁ in serum erroneous for evaluation of TGF-β₁ originated from or induced by the prostate. To ensure complete platelet removal, an additional centrifugation was performed in the present study, as recommended by Adler et al. (1999), and almost identical amounts of plasma TGF-β₁ were observed. While, as expected, TGF-β₁ values in the serum format were only weakly correlated with those in the plasma formats (correlation coefficients, 0.79 and 0.80), the plasma formats were strongly correlated with each other (correlation coefficient, 0.99). The 2-times lower TGF-β₁ values obtained with the citrate plasma as compared to the EDTA plasma collection format may be due to dilution of the top plasma layer primarily by 1.0 mL of 0.1 mol/L sodium citrate anticoagulant, in the Vacutainer®CPT™ tubes.

This study was limited by the low rate of disease progression in the patient cohort (14%) after a median follow-up of 53.8 months, yielding an estimated 5 year progression-free probability of 85%. The low progression rate in the above-described population may be due to the lower cancer stage and volume observed in more recent surgical series that has accompanied the increasing awareness of prostate cancer in the general population and the wide availability of PSA based screening (Farkas et al., 1998). In other reported series, approximately 44% to 47% of men undergoing radical prostatectomy had pathologically non-organ-confined disease (Partin et al., 1993; Wheeler et al., 1998), while in the present cohort, only 34.2% of cancers were not organ-confined. The pathologic stage of prostate cancer is known to be a strong predictor of progression after radical prostatectomy (Epstein et al., 1996). Nevertheless, 92.5% of the present patients had a pre-operative PSA level above 4 ng/mL; 32.5% had extraprostatic extension in their pathologic prostatectomy specimen, and 50% had a final pathological Gleason score of 7 and above, representative of patients undergoing radical prostatectomy for clinically localized prostate cancer. In addition to a slightly more favorable profile in pathological parameters in the above-described study cohort, the lower progression rate may be due to differences in surgical technique (Ohori et al., 1995; Epstein et al., 1996). The positive margin rate in the present series was 13.3% compared with the 16% to 46% positive margin rates reported by others in patients with clinically localized prostate cancer (Ohori et al., 1995; Jones, 1990), which may have decreased the rate of progression due to local failure.

In conclusion, plasma TGF-β₁ levels are markedly elevated in men with prostate cancer metastatic to regional lymph nodes and bone. In men without clinical or pathological evidence of metastases, the pre-operative plasma TGF-β₁ level is the strongest predictor of biochemical progression after surgery likely due to an association with occult metastatic disease present at the time of radical prostatectomy.

### EXAMPLE 2

Materials and Methods

Patient Population

Plasma IGF-I, IGF BP-2, and IGF BP-3 levels were assessed in 44 healthy patients without cancer, in 19 men with prostate cancer metastatic to regional lymph nodes, and in 10 patients with bone scan-proven, metastatic prostate cancer. Neither patients with metastatic lymph node disease nor patients with metastatic bone disease were treated with either hormonal or radiation therapy before plasma collection. The healthy non-cancer group was composed of three sets of consecutive patients who participated in a weekly prostate cancer screening program. They had no prior history of any cancer or chronic disease, a normal digital rectal examination, and a PSA of less than 2.0 ng/mL, a PSA range that has an estimated probability of prostate cancer detection of less than 1% in the first 4 years after screening (Smith, 1996).

Also, 120 consecutive patients were studied who underwent radical prostatectomy for clinically localized prostatic adenocarcinoma (clinical stage T1 to T2) and who had available plasma samples. No patient was treated pre-operatively with either hormonal or radiation therapy, and none had any secondary cancer. The clinical stage was assigned by the operative surgeon according to the 1992 TNM system. The mean patient age in this study was 61.8±7.2 years (median 63.0, range 40 to 76). Serum prostate specific antigen was measured by the Hybritech® Tandem-R assay (Hybritech, Inc., San Diego, Calif.).

IGF-I, IGF BP-2, and IGF BP-3 Measurements

Serum and plasma samples were collected on an ambulatory basis at least 4 weeks after transrectal guided needle biopsy of the prostate, typically performed on the morning of the scheduled day of surgery after a typical pre-operative overnight fast. Blood was collected into Vacutainer® CPT™ 8 mL tubes containing 0.1 mL of 1 M sodium citrate anticoagulant (Becton Dickinson Vacutainer Systems, Franklin Lakes, N.J.) and centrifuged at room temperature for 20 minutes at 1500×g. The top layer corresponding to plasma was decanted using sterile transfer pipettes and immediately frozen and stored at −80° C. in polypropylene cryopreservation vials (Nalge Nunc, Rochester, N.Y.). For quantitative measurements of serum and plasma IGF-I and IGF BP-3 levels, the DSL-10-5600ACTIVE®IGF-I Elisa kit and the DSL-10-6600ACTIVE®IGF BP-3 Elisa kit were used, respectively (DSL, Webster, Tex.). For quantitative measurements of serum and plasma IGF BP-2 levels, the DSL-7100 IGF BP-2 Radioimmunoassay kit (DSL) was used. Every sample was run in duplicate, and the mean was used for data analysis. Differences between the two measurements were minimal, as shown the intra-assay precision coefficient of variation of only 4.73±1.87% for IGF-1,6.95±3.86% for IGF BP-2, and 8.78±4.07 for IGF BP-3.

IGF BP-2 and IGF BP-3 Collection Formats

In a preliminary study, IGF BP-2 and IGF BP-3 levels were assessed in three synchronously drawn blood specimens obtained from 10 of the 44 healthy screening patients. Plasma was separated using Vacutainer® K₃ ethylenediaminetetraacetic acid (EDTA) 5 mL tubes containing 0.057 mL of 15% K₃ EDTA solution, and Vacutainer® CPT™ 8 mL tubes containing sodium citrate (Becton Dickinson Vacutainer Systems, Franklin Lakes, N.J.). Serum was separated using Vacutainer® Brand SST Serum Separator™ tubes (Becton Dickinson Vacutainer Systems, Franklin Lakes, N.J.). Specimens were centrifuged at room temperature for 20 minutes at 1500×g, and plasma or serum decanted and frozen at −80° C. until assessment. The investigators were blinded to the nature of the collection formats. Analysis of variance was used to determine whether the collection format significantly affected measured IGF BP-2 and IGF BP-3 levels.

Pathological Examination

All prostatectomy specimens were examined pathologically by a single pathologist who was blinded to clinical outcome. Pelvic lymph nodes were removed in a standard fashion at surgery and examined microscopically for the presence of metastatic prostate cancer. The radical prostatectomy specimens were processed by whole-mount technique, and pathological parameters evaluated as previously described (Wheeler, 1994).

Post-Operative Follow-Up

Each patient was scheduled to have a digital rectal examination and serum PSA post-operatively every 3 months for the first year, semiannually from the second through the fifth year, and annually thereafter. A staging evaluation, including bone scan, prostascint, and/or PSA doubling time calculation was performed in 11 of the 15 patients who had PSA progression prior to the administration of salvage radiation or hormonal therapy. Biochemical progression was defined as a sustained elevation, on 2 or more occasions, of PSA>0.2 ng/mL. The date of progression was assigned to the date of the first value >0.2 ng/mL. Two (1.7%) patients had lymph node positive disease at the time of radical prostatectomy, and surgery was consequently aborted prior to prostate removal. These patients were categorized as failures from the day after surgery. Two (1.7%) patients received adjuvant radiation therapy before biochemical progression because of positive surgical margins. One of them subsequently experienced PSA relapse and was categorized as having progression from the date of the first value >0.2 ng/mL, while the second was censored on the date of the last follow-up examination. There were 17 failures among the 120 men. PSA relapse was the sole indication of progression in 14 patients, while 3 had clinical, as well as biochemical evidence of progression.

Statistical Analysis

Differences in plasma IGF BP-2 and IGF BP-3 levels were assessed using analysis of variance (ANOVA). Multiple comparisons were conducted, when the overall test was significant (one-way ANOVA followed by Fisher's least significant difference). Spearnan's rank correlation coefficient was used to compare ordinal and continuous variables. Logistic regression was used for multivariate analysis of binary outcome variables. The Kaplan-Meier method was used to calculate survival functions, and differences were assessed with the long rank statistic. Multivariate survival analysis was performed with the Cox proportional hazard regression model. Pre-operative PSA level had a skewed distribution and therefore was modeled with a log transformation. Clinical stage was evaluated as TI versus T2 and biopsy Gleason score was evaluated as grade 2 to 6 versus grade 7 to 10. Statistical significance in this study was set as P<0.05. All reported P values are two-sided. All analyses were performed with SPSS statistical package (SPSS version 10.0 for Windows).

Results

Impact of Collection Formats on IGF BP-2 and IGF BP-3 Levels

Initially, the effect of the collection format on IGF BP-2 and IGF BP-3 levels was studied. Mean IGF BP-2 and IGF BP-3 levels, measured in Vacutainer®CPT™ citrate plasma, Vacutainer®K₃ EDTA plasma, and Vacutainer®BrandSST™ serum from synchronously drawn blood specimens of 10 consecutive, healthy screening patients are shown in Table 7. IGF BP-2 and IGF BP-3 levels measured in citrate plasma were 26% and 28%, respectively, lower than those measured in EDTA plasma, and 37% and 39%, respectively, lower than those measured in serum. Although analysis of variance showed IGF BP-2 and IGF BP-3 inter-collection format differences to be statistically significant (P values <0.001), IGF BP-2 and IGF BP-3 levels measured in specimens collected by all three sample formats were found to be highly correlated with each other (P values <0.001). Similarly to previous results on IGF-I (Shariat, 2000), while statistically significant differences were found in absolute IGF BP-2 and IGF BP-3 levels measured in different collection formats, all three collection formats were highly correlated with each other. Plasma from Vacutainer®CPT™ sodium citrate tubes was used for IGF-I, IGF BP-2, and IGF BP-3 measurements in the following study.

TABLE 7

| Collection FormatError! | IGF BP-2 (ng/mL) | | IGF BP-3 (ng/mL) | |
|---|---|---|---|---|
| Bookmark not defined. | Mean | SD* | Mean | SD* |
| Citrate plasma | 359.3 | 18.1 | 3273 | 256 |
| EDTA plasma | 487.9 | 28.4 | 4566 | 376 |
| Serum | 567.8 | 31.0 | 5401 | 430 |
| | P | Correlation | P | Correlation |
| Collection Formats | value† | Coefficient‡ | value† | Coefficient‡ |
| EDTA plasma and citrate | <0.001 | 0.79 | <0.001 | 0.81 |
| plasma | | | | |
| EDTA plasma and serum | <0.001 | 0.70 | <0.001 | 0.72 |
| Citrate plasma and serum | <0.001 | 0.73 | <0.001 | 0.78 |

*SD = Standard Deviation.
†P-values (two-sided) were calculated based on analysis of variance in a randomized complete block design for the assessment of the difference in IGF BP-2 and IGF BP-3 levels between collection formats.
‡Spearman correlation coefficients were used to assess the relationship between different collection formats.

Clinical and Pathological Characteristics

All patients had clinically localized (T1 or T2) disease, and the mean pre-operative TGF-β₁ and PSA levels were 5.4±2.0 ng/mL (median 4.9, range 1.66 to 15.1) and 9.5±6.3 ng/mL (median 8.2, range 2.1 to 49.0), respectively. Nine (7.5%) patients had PSA levels less than 4 ng/mL; 75 (62.5%) had PSA levels greater than or equal to 4 ng/mL and less than 10 ng/mL; and 36 (30.0%) had PSA levels greater than or equal to 10 ng/mL. Clinical and pathological characteristics are listed in Table 8. On univariate analysis (Table 9), pre-treatment IGF BP-2 levels correlated with pathological stage (P<0.001) and grade (P=0.025) and IGF BP-3 levels correlated with IGF-1 levels (P<0.001).

TABLE 8
Pre-Operative Characteristics

| | Patients | | Patients |
|---|---|---|---|
| Clinical stage | N (%) | Biopsy Gleason score | N (%) |
| cT1 a + b | 1 (0.8) | 2-4 | 3 (2.5) |
| cT1 c | 41 (34.2) | 5-6 | 77 (64.2) |
| cT2 a | 46 (38.3) | 7 | 35 (29.2) |
| cT2 b | 16 (13.3) | 8-10 | 5 (4.1) |
| cT2 c | 16 (13.3) | | |

Post-Operative Characteristics

| | Patients | Pathologic Gleason | Patients |
|---|---|---|---|
| Pathological features | N (%) | score* | N (%) |
| Organ Confined | 79 (65.8) | 2-4 | 0 (0) |
| ECE only | 33 (27.5) | 5-6 | 59 (50.0) |
| SVI+ | 8 (6.7) | 7 | 56 (47.5) |
| LN+ | 2 (1.7) | 8-10 | 3 (2.5) |
| SM+ | 16 (13.3) | | |

ECE = Extracapsular extension.
SVI+ = Seminal vesicle invasion.
LN+ = Lymph node positive.
SM+ = Positive surgical margins.
*Gleason tumor grade unavailable for two patients, who did not undergo a prostatectomy because of grossly positive pelvic lymph nodes at the time of surgery.

TABLE 9

| | Pre-Operative IGF BP-2 | | Pre-Operative IGF BP-3 | |
|---|---|---|---|---|
| | Correlation | | Correlation | |
| Parameter | Coefficient* | P value | Coefficient* | P value |
| Age | 0.092 | 0.316 | −0.066 | 0.472 |
| Pre-operative PSA | 0.064 | 0.490 | −0.131 | 0.153 |
| Pre-operative IGF-I | 0.013 | 0.884 | 0.61 | <0.001 |
| Clinical stage | −0.009 | 0.921 | 0.056 | 0.546 |
| Biopsy Gleason sum | −0.204 | 0.025 | −0.071 | 0.438 |
| Final pathologic | −0.375 | <0.001 | −0.104 | 0.261 |
| stage | | | | |
| Final pathologic | −0.204 | 0.027 | −0.013 | 0.891 |
| Gleason sum | | | | |

*Spearman's correlation coefficients were used to assess the relationship of IGF BP-2 and IGF BP-3 levels with IGF-I levels and clinico-pathological parameters.

Final Pathological Stage and Progression as a Function of IGF BP-2 and IGF BP-3 and Other Parameters

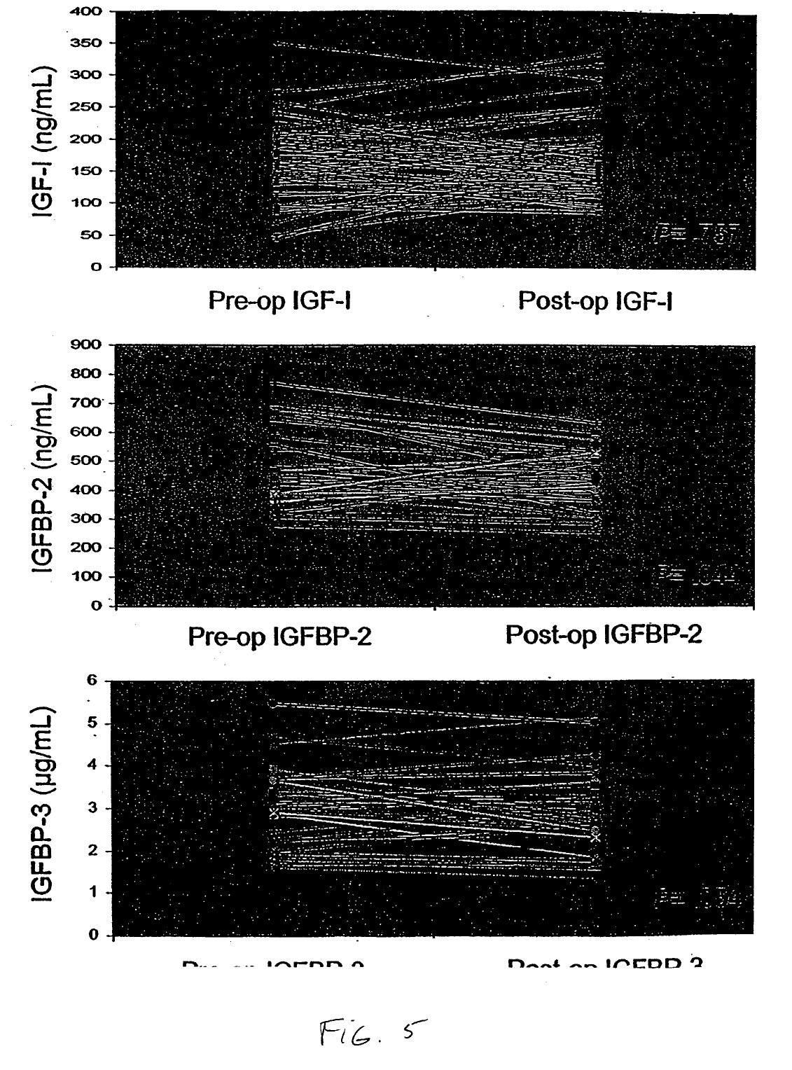

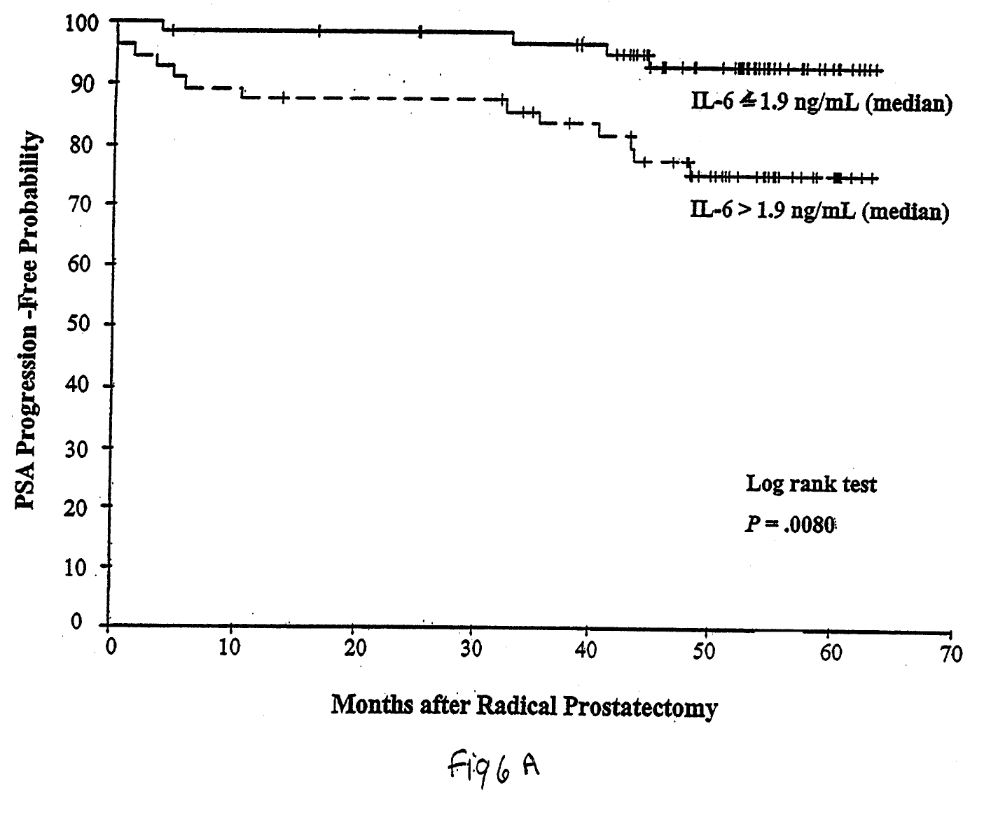

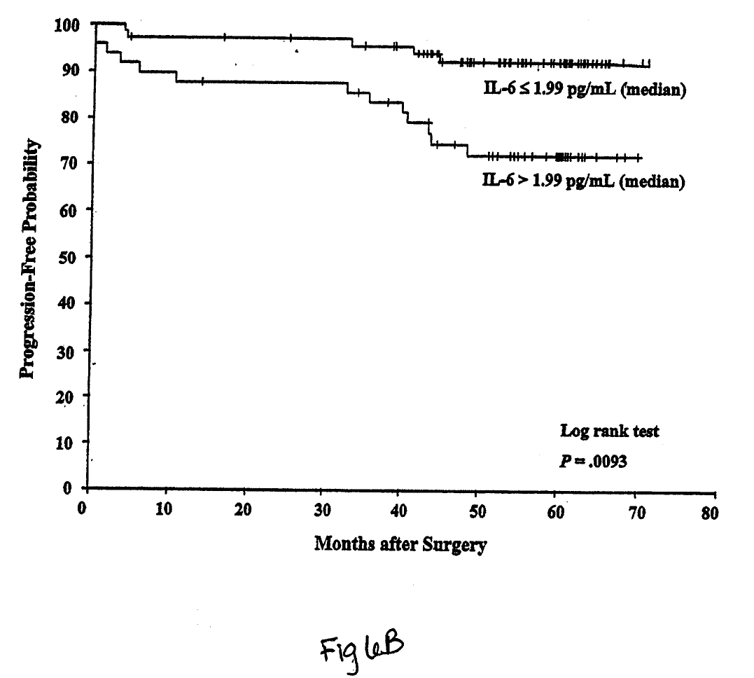

In a multivariate logistic regression analysis, pre-operative plasma IGF BP-2 levels (P=0.001), pre-operative serum PSA levels (P=0.034), and biopsy Gleason grade (P=0.005) were significant predictors of organ-confined disease. Overall, only 14% of patients (17 of 120) had cancer progression with a median post-operative follow-up of 53.8 months (range 1.16 to 63.3). The overall PSA progression-free survival was 90.7±5.3% (95% CI) at 3 years and 84.6±6.8% (95% CI) at 5 years. Using the log rank test, it was found that patients with pre-operative plasma IGF BP-2 levels below the median (437.4 ng/mL) had a significantly increased probability of PSA-progression (P=0.0310; FIG. 3). However, there was no significant difference in PSA-progression-free survival (FIG. 4) between patients stratified by the median level of IGF BP-3 (3239 ng/mL; P=0.0587). On univariate Cox proportional hazards regression analysis (Table 10), plasma IGF BP-2 was associated with the risk of PSA progression (P=0.015) along with biopsy Gleason score (P=0.005). In a pre-operative multivariate model that included pre-operative IGF BP-2, pre-operative PSA, clinical stage, and biopsy Gleason score, plasma IGF BP-2 level and biopsy Gleason score were both independent predictors of disease progression (P=0.049 and P=0.035, respectively). In alternative models where IGBP-2 was replaced by IGF-I, IGF BP-3, or both, biopsy Gleason score was the sole independent predictor of PSA progression (P values <0.09). However when IGF BP-3 level was adjusted for IGF BP-2 level, IGF BP-3 became an independent predictor of disease progression (P values <0.040) and the association of IGF BP-2 with the risk of prostate progression strengthened (P values <0.039). When all three, IGF-I, IGF BP-2, and IGF BP-3 were adjusted for each other, IGF BP-2, IGF BP-3, and biopsy Gleason score were independent predictors of disease progression (P=0.031, P=0.035, and P=0.036, respectively; Table 10).

TABLE 10

| | Univariate | | | Multivariate | | |
|---|---|---|---|---|---|---|
| | Hazard | | | Hazard | | |
| Variable | ratio | P | 95% CI | ratio | P | 95% CI |
| Pre-Operative | 0.997 | 0.490 | 0.990-1.005 | 1.003 | 0.454 | 0.995-1.012 |
| IGF-I levels | | | | | | |
| Pre-Operative IG | 0.993 | 0.015 | 0.988-0.999 | 0.994 | 0.031 | 0.988-0.999 |
| FBP-2 levels | | | | | | |
| Pre-Operative IG | 0.946 | 0.53 | 0.895-1.001 | 0.926 | 0.035 | 0.836-0.995 |
| FBP-3 levels | | | | | | |
| Pre-Operative PSA | 5.772 | 0.067 | 0.887-37.547 | 3.671 | 0.124 | 0.699-19.270 |
| levels* | | | | | | |
| Biopsy Gleason | 4.167 | 0.005 | 1.541-11.273 | 3.055 | 0.036 | 1.079-8.654 |
| Score† | | | | | | |
| Clinical Stage‡ | 1.850 | 0.226 | 0.684-5.002 | 1.769 | 0.293 | 0.611-5.122 |

*Pre-operative PSA levels were logarithmically transformed.
†Biopsy Gleason Score was categorized as grade 2 to 6 versus grade 7 to 10.
‡Clinical stage was categorized as T1 versus T2.

Characteristics of Patients with Disease Progression

Of the 17 radical prostatectomy patients who progressed, two (12%) patients had lymph node positive disease at the time of radical prostatectomy. Five patients were presumed to have local failure because their PSA doubling times were greater than 12 months (n=3; median 19.6, range 15.8-21.6) or because they achieved a complete response to local salvage radiation therapy (n=2). Eight patients were presumed to have distant failure because of the results of a metastatic work-up (positive bone scan or prostascint; n=3), because their PSA doubling times were less than 10 months (n=7; median 6.6, range 1.97-9.80), or because they failed to respond to local radiation therapy (n=4). Pre-operative plasma IGF-I levels, IGF BP-2 levels, and IGF BP-3 levels were not significantly different in patients with presumed distant failure than those with local failure (P=0.898, P=0.600, and P=0.059, respectively).

IGF BP-2 and IGF BP-3 in Healthy and Metastatic Patients

Plasma IGF-I levels in 19 patients with prostate cancer metastatic to regional lymph nodes (median 156 ng/mL, range 100-281), in the 10 patients with prostate cancer metastatic to bones (153 ng/mL, range 29-360), in the cohort of 120 prostatectomy patients (median 151 ng/mL, range 42-451), and in the 44 healthy screening patients (median 171 ng/mL, range 62-346) were not significantly different from each other (P=0.413). However, plasma IGF BP-2 levels in the prostatectomy patients (median 437 ng/mL, range 209-871), in the patients with lymph node metastases (median 437 ng/mL, range 299-532), and in the patients with bone metastases (median 407 ng/mL, range 241-592) were significantly higher then those in the healthy subjects (median 340 ng/mL, range 237-495; P values <0.006). Plasma IGF BP-2 levels in patients with clinically localized prostate cancer, with lymph node metastases, or with bone metastases were not significantly different from each other (P values >0.413). Plasma IGF BP-3 levels in patients with lymph node metastases (median 2689 ng/mL, range 1613-3655) and bone metastases (median 2555 ng/mL, range 1549-3213) were significantly lower than those in the cohort of 120 prostatectomy patients (median 3217 ng/mL, range 1244-5452) and in healthy subjects (median 3344 ng/mL, range 1761-5020; P values <0.031). However, plasma IGF BP-3 levels in the prostatectomy patients were not significantly different than those in healthy subjects (P=0.575).

Discussion

IGF BP-2 levels were elevated in patients with non-metastatic and metastatic prostate cancer compared to levels in healthy subjects. A significant association was found between pre-operative plasma IGF BP-2 levels and established markers of biologically aggressive prostate cancer, such as final pathologic stage and grade in patients with clinically localized prostate cancer. Furthermore, pre-operative plasma IGF BP-2 was a robust independent predictor of final pathologic stage and disease progression in a large cohort of consecutive patients with long term follow-up after radical prostatectomy. However, in patients that progressed, pre-operative plasma IGF BP-2 levels were not significantly different in patients with presumed distant failure than those with presumed local-only failure. Plasma IGF BP-3 levels were significantly lower in patients with prostate cancer metastatic to regional lymph nodes and to bones compared to levels in patients with non-metastatic prostate cancer and healthy subjects. While no significant association was found between pre-operative plasma IGF BP-3 levels and established markers of biologically aggressive prostate cancer or disease progression, when adjusted for IGF BP-2 levels, plasma IGF BP-3 was independently associated with prostate cancer progression.

Circulating IGF BP-2 levels are not correlated to circulating IGF-I levels, since more than 90% circulating IGF-I molecules are complexed with IGF BP-3 and a glycoprotein named acid-labile subunit. Most of the circulating IGF-I and IGF BP-3 are produced by the liver and growth hormone stimulates both IGF-I and IGF BP-3 production (Jones, 1995). This growth hormone regulated hepatic release of both IGF-I and IGF BP-3 may explain in part the highly significant but moderate correlation (r=0.61) that was found. Other studies have found an almost identical correlation coefficient.

PSA is an IGF BP-3 protease, capable of acting as a co-mitogen with IGFs in the presence of IGF BP-3 (Cohen, 1992). IGF BP-3 proteolysis by PSA (Cohen, 1994) and cathepsin D (Nunn et al., 1997) likely signify local effects rather then systemic effects, within the prostate or metastatic foci leading to local progression or metastasis growth. Elevated serum PSA level has been correlated with decreased IGF BP-3 (Kanety, 1993).

IGF-I and BPH increase in follow-up doubling the number of cancer-free controls, as well as measurements of IGF-I levels in patients with regional lymph node metastases. Previously, no association was found between circulating IGF-I levels and established markers of biologically aggressive prostate cancer, disease progression, or metastasis. Various independent studies have found no difference in IGF-I levels between patients with prostate cancer and healthy men. Furthermore, a recent study investigating IGF-I levels in a PSA-based screening positive population found IGF-I not to be a useful marker for prostate cancer screening and concluded that high circulating IGF-I level is more likely related to BPH and prostatic enlargement (Finne, 2000), but may be related to prostate cancer risk (early, subclinical disease), but not to cancer biology and prognosis, which more likely results in the disruption of the cellular physiology of IGFs or other growth factors.

While prostate cancer incidence is not increased in patients with acromegaly, the incidence of BPH or enlarged prostate is (Coalo, 1998). Patients with elevated growth hormone who were successfully treated had normal prostate volume and growth hormone deficient subjects had reduced prostate volume. Moreover, IGF-I has been shown to stimulate the growth of BPH derived stromal cells in vitro (Sutkowski et al., (1999).

The mean IGF BP-2 and IGF BP-3 levels measured in Vacutainer®CPT™ citrate plasma were 26% and 28%, respectively, lower than those measured in Vacutainer®K₃EDTA plasma, and 37% and 39%, respectively, lower than those measured in Vacutainer®BrandSST serum. The consistent in relative differences measured between the three collection formats for each assay, and the resemblance to relative difference of 27% and 42% for IGFF-I found previously (Shariat, 2000), support that the measurement technique employed was consistent and that the levels of the relative changes of the three markers can be compared. Furthermore this supports that the lower IGF-I, IGF BP-2, and IGF BP-3 values obtained with the Vacutainer®CPT™ citrate plasma as compared to the Vacutainer®K₃EDTA plasma collection format are due to dilution of the top plasma layer primarily by 1.0 mL of 0.1 M sodium citrate anticoagulant. However, although there were statistically significant differences in absolute IGF-I, IGF BP-2, and IGF BP-3 levels measured in serum and in plasma using different collection formats, all three are highly correlated with each other and therefore equally valid as long as the same collection format is used throughout the study.

The complex nature of the IGF axis may require simultaneous measurement of multiple factors in order to fully appreciate the biologic activity of this system. Measurement of other IGF BPs may add to the biological relevance of IGFs in prostate cancer. Other IGF BPs, such as IGF BP-4 and IGF BP-5 have been associated with tumor grade in prostate specimens, and with tumor stage and serum PSA levels in patients. Equally important, IGF-I receptor mediates most of the mitogenic effects of IGFs, and experimental inhibition of the IGF-I receptor has resulted in suppression of adhesion, invasion, and metastases in prostate cancer (Kaplan, 1999). Recent studies suggest that circulating levels of IGFs may not be determinants of tissue bioactivity but rather may vary in parallel with autocrine or paracrine expression within tissues (Yakar, 1999). Since hepatic IGF-I and IGF BP-3 are the major contributors of circulating levels of these two IGFs, important autocrine and paracrine production occurring in other tissues such as the prostate may not be reflected by changes in systemic levels of these molecules.

In conclusion, plasma IGF BP-2 levels are markedly elevated in men with prostate cancer. In men without clinical or pathological evidence of metastases, the pre-operative plasma IGF BP-2 level is a robust predictor of final pathologic stage and biochemical progression after surgery. This association seems, however, not to be due to an association with occult metastatic disease present at the time of radical prostatectomy. On the contrary, pre-operative circulating IGF BP-3 and IGF-I levels are not independently associated with established markers of biologically aggressive prostate cancer or PSA progression-free survival. The lack of any association with markers of more aggressive prostate cancer or with prostate cancer progression may limit the clinical utility of IGF-I and IGF BP-3 as tumor markers for prostate cancer.

### EXAMPLE 3

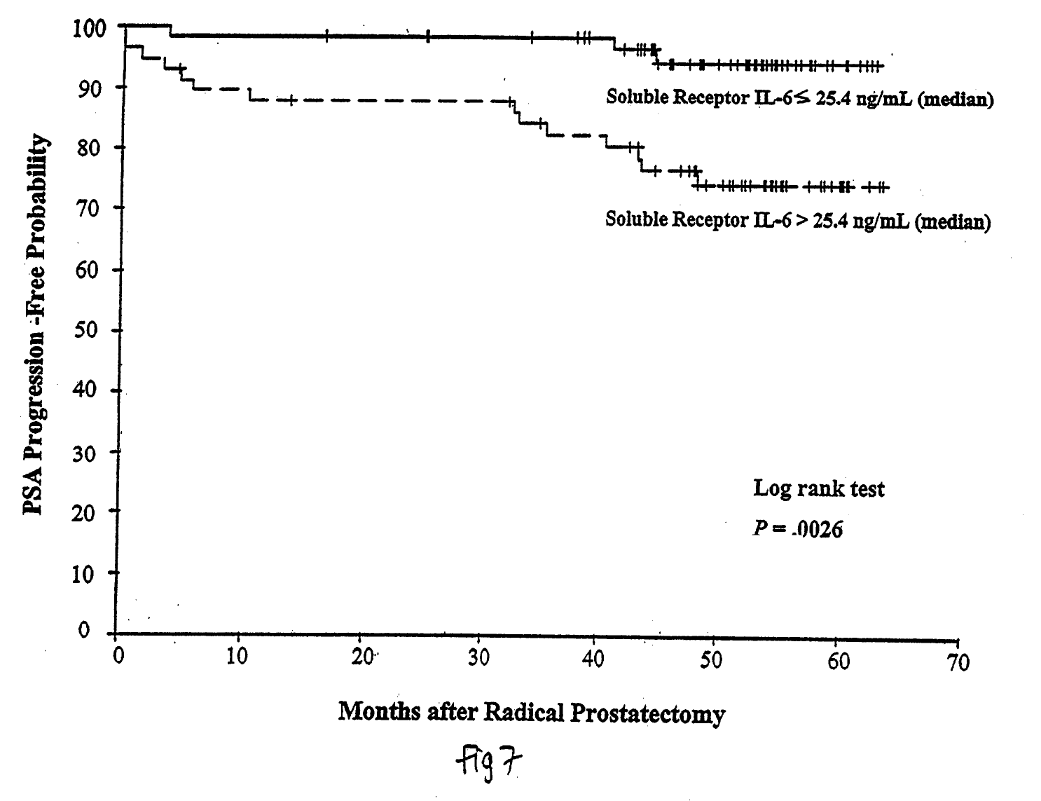

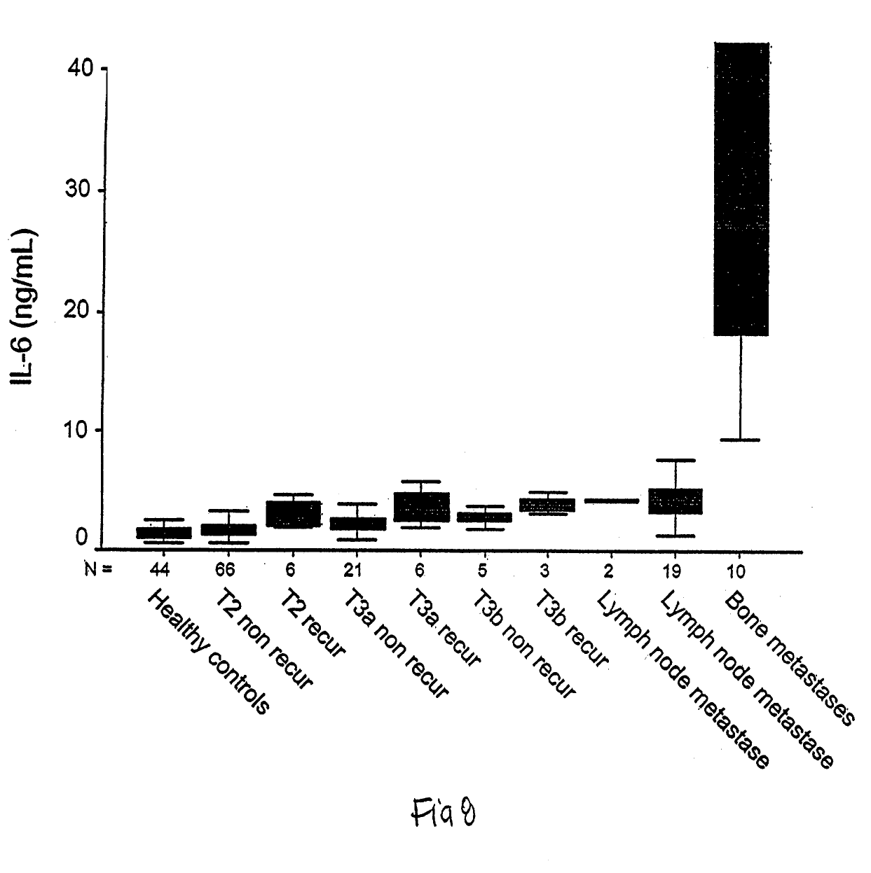

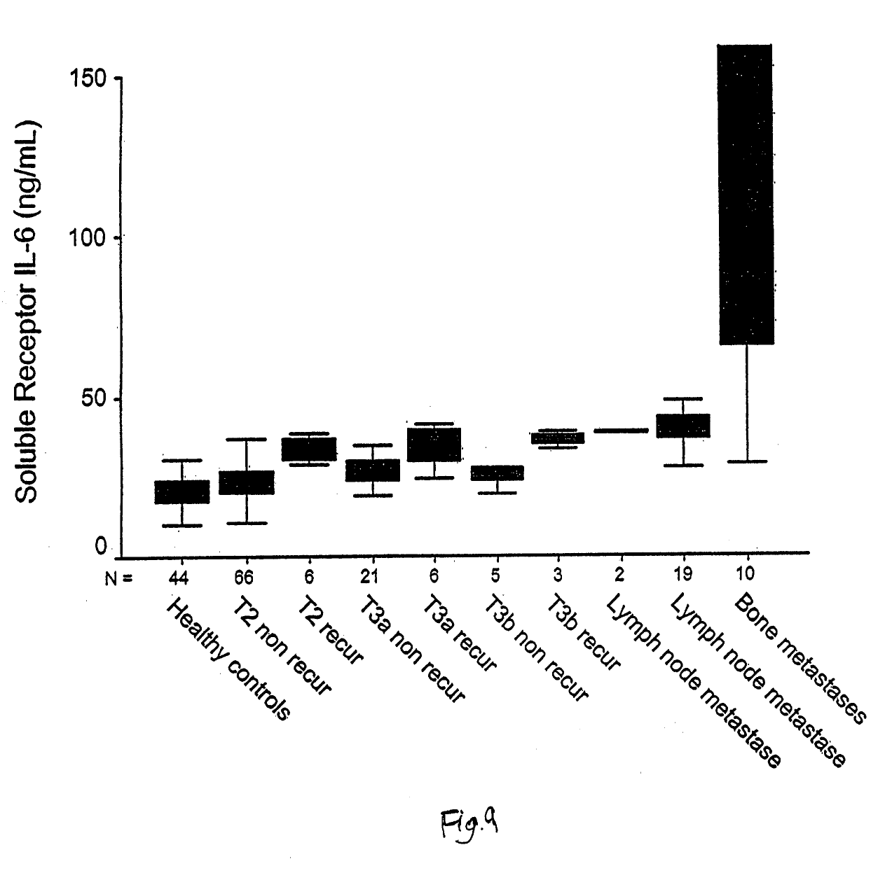

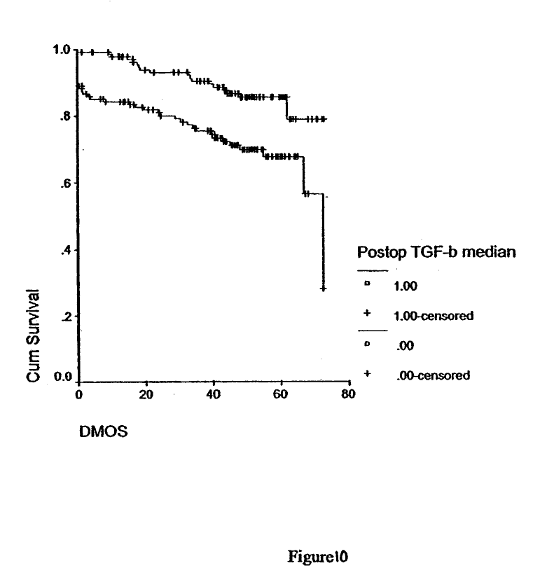

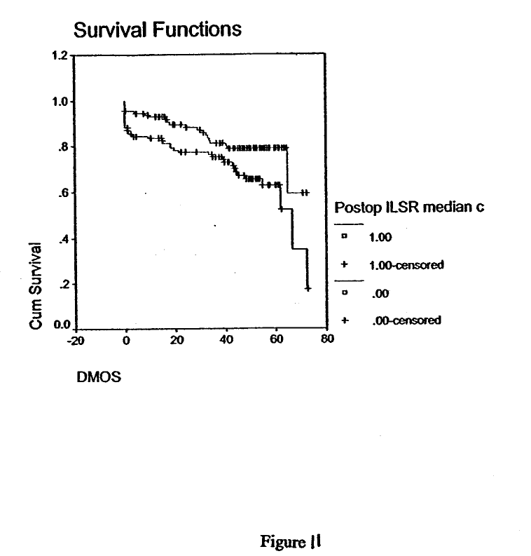

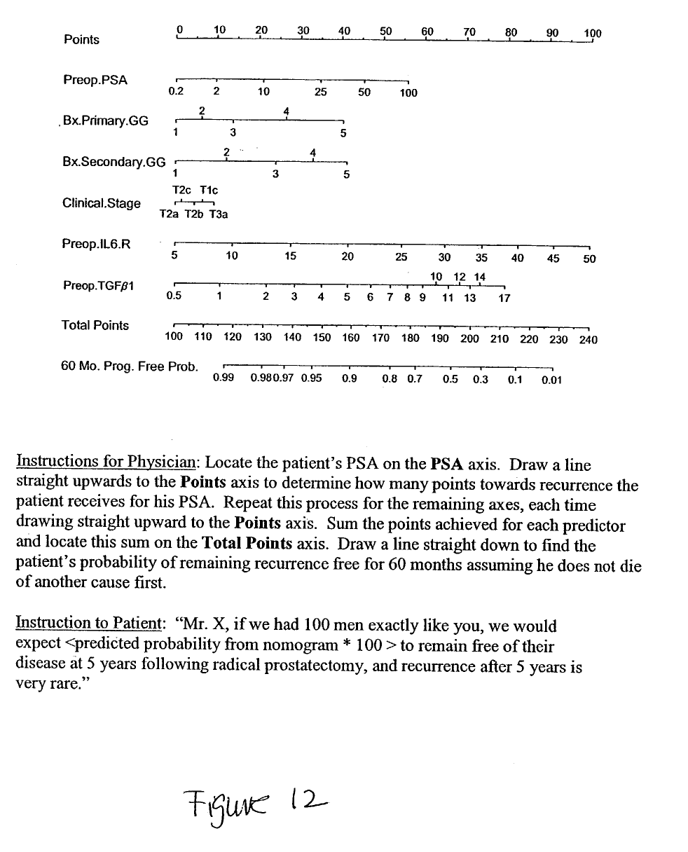

A similar analysis was conducted for IL-6 and IL6sR (using R&D Systems Quantikine kits for IL-6 and IL6sR, catalog numbers DR6050 and DR600, respectively) and it was found that the pre-operative plasma levels of IL-6 and IL6sR were correlated with clinical and pathological parameters in the 120 patients who underwent radical prostatectomy (FIGS. 6-9 and Tables 11-12). Plasma IL-6 and IL6sR levels in patients with bone metastases were significantly higher than those in healthy subjects, in prostatectomy patients, or in patients with lymph node metastases (P values <0.001). In a pre-operative model that included IL-6 or IL6sR in addition to Partin nomogram variables, pre-operative plasma IL-6, IL6sR, and biopsy Gleason score were independent predictors of organ-confined disease (P values <0.01) and PSA progression (P values <0.028). In an alternative model that included both IL-6 and IL6sR, only pre-operative plasma IL6sR remained an independent predictor of PSA progression (P=0.038). Thus, IL-6 and IL6sR levels are elevated in men with prostate cancer metastatic to bone. In patients with clinically localized prostate cancer, the pre-operative plasma level of IL-6 and IL6sR are associated with markers of more aggressive prostate cancer and are predictors of biochemical progression after surgery.

TABLE 11
Pre-Operative Features

| | Univariate | | | Multivariate | | |
|---|---|---|---|---|---|---|
| | Hazard | | | Hazard | | |
| | ratio | P | 95% CI | ratio | P | 95% CI |
| Pre-Operative | 5.772 | 0.067 | 0.887-37.547 | 4.197 | 0.131 | 0.652-27.017 |
| PSA levels* | | | | | | |
| Pre-Operative IL-6 | 2.291 | <0.001 | 1.678-3.128 | 1.226 | <0.001 | 1.114-1.3498 |
| levels | | | | | | |
| Biopsy Gleason | 4.167 | 0.005 | 1.541-11.273 | 2.063 | 0.185 | 0.707-6.020 |
| Sum† | | | | | | |
| Clinical Stage‡ | 1.850 | 0.226 | 0.684-5.002 | 1.085 | 0.977 | 0.347-2.798 |

*Pre-operative PSA levels were logarithmically transformed.
†Biopsy Gleason sum was categorized as grade 2 to 6 versus grade 7 to 10.
‡Clinical stage was categorized as T1 versus T2.

TABLE 12
Pre-Operative Features

| | Univariate | | | Multivariate | | |
|---|---|---|---|---|---|---|
| | Hazard | | | Hazard | | |
| | ratio | P | 95% CI | ratio | P | 95% CI |
| Pre-Operative | 5.772 | 0.067 | 0.887-37.547 | 7.083 | 0.044 | 1.051-47.726 |
| PSA levels* | | | | | | |
| Pre-Operative IL-6 | 1.260 | <0.001 | 1.154-1.375 | 2.174 | <0.001 | 1.550-3.048 |
| levels | | | | | | |
| Biopsy Gleason | 4.167 | 0.005 | 1.541-11.273 | 3.218 | 0.026 | 1.148-9.025 |
| Sum† | | | | | | |
| Clinical Stage‡ | 1.850 | 0.226 | 0.684-5.002 | 1.135 | 0.814 | 0.396-3.254 |

*Pre-operative PSA levels were logarithmically transformed.
†Biopsy Gleason sum was categorized as grade 2 to 6 versus grade 7 to 10.
‡Clinical stage was categorized as T1 versus T2.

### EXAMPLE 4

Subjects and Methods

Patient Population

All studies were undertaken with the approval and institutional oversight of the Institutional Review Board for the Protection of Human Subjects at Baylor College of Medicine. All 511 patients admitted to The Methodist Hospital with the intent to treat their clinically localized prostate cancer (cT1c-3a, NX, MO) with radical prostatectomy by surgeons of the Scott Department of Urology were potential candidates for this analysis. The clinical stage was assigned by the operative surgeon according to the 1992 TNM system. After obtaining consent, pre- and post-operative plasma specimens were obtained for 357 of these men. Thirty-five men initially treated with hormonal therapy, 11 who were treated with definitive radiotherapy, and 2 who were treated with cryotherapy before surgery, were excluded from the analysis. No disease follow-up information was available for 7 men, and they were also excluded. This left 302 men for analysis. The mean patient age in this study was 61.8±7.3 y (median 62.6, range 40 to 80). Serum prostate specific antigen was measured by the Hybritech® Tandem-R assay (Hybritech, Inc., San Diego, Calif.).

TGF-β₁, IL-6 and IL6sR Measurements

Pre-operative serum and plasma samples were collected at least 4 weeks after transrectal guided needle biopsy of the prostate, typically on the morning of the day of surgery after an overnight fast. Post-operative plasma samples were collected between 6 and 8 weeks after surgery. Specimen collection and measurement was described previously in Shariat et al. (2001a) and Shariat et al. (2001b). Briefly, blood was collected into Vacutainer® CPT™ 8 mL tubes containing 0.1 mL of 1 M sodium citrate (Becton Dickinson, Franklin Lakes, N.J.) and centrifuged at room temperature for 20 minutes at 1500×g. The top layer corresponding to plasma was decanted using sterile transfer pipettes and immediately frozen and stored at −80° C. in polypropylene cryopreservation vials (NalgeNunc, Rochester, N.Y.). For quantitative measurements of TGF-β₁, IL-6 and IL6sR levels, quantitative immunoassays were used (R&D Systems, Minneapolis, Minn.). Previously, it was found that TGF-β₁ levels were 3 to 6-times higher when measured in serum than when measured in plasma (Shariat et al., 2001b). Since TGF-β₁ is present in platelet granules and is released upon platelet activation, the higher levels of TGF-β₁ in serum were likely due at least in part to release from damaged platelets, making the quantification of non-platelet derived TGF-β₁ less accurate. Therefore, as in the previous study, for TGF-β₁, prior to assessment, an additional centrifugation step of the plasma was performed at 10,000×g for 10 minutes at room temperature for complete platelet removal. Every sample was run in duplicate, and the mean was used. Differences between the two measurements for TGF-β₁, IL-6 and IL6sR were minimal (intra-assay precision coefficients of variation: 5.43±2.01%, 4.37±2.39%, and 4.98±3.24%, respectively).

Pathologic Examination

All prostatectomy specimens were examined pathologically by a single pathologist, who was blinded to clinical outcome. The radical prostatectomy specimens were processed by whole-mount technique, and pathological parameters were evaluated in a manner previously described by Wheeler et al. (1994). Total tumor volume was computed by computerized planimetry from the whole-mount sections for 255 of the 302 prostatectomy patients (Greene et al., 1991).

Post-Operative Follow-Up

Patients generally were scheduled to have a digital rectal examination and serum PSA evaluation post-operatively every 3 months for the first year, semiannually from the second through the fifth year, and annually thereafter. Biochemical progression was defined as a sustained elevation, on 2 or more occasions, of PSA>0.2 ng/mL and was assigned to the date of the first value >0.2 ng/mL. Pelvic lymph node dissections were performed on all men. Radical prostatectomy was aborted in two of the six patients who were found to have nodal metastases on frozen section analysis during the operation; these men are not excluded from the analysis. All patients with metastases to regional lymph nodes were categorized among those with progression from the day after surgery. Six patients (2%) received adjuvant radiation therapy before biochemical progression because of positive surgical margins. Three of them subsequently experienced PSA relapse and were considered to have disease progression from the date of the first value >0.2 ng/mL, while the other three were censored on the date of the last follow-up examination. Of 302 patients who underwent radical prostatectomy, 43 had progression of disease. A staging evaluation, including bone scan, Prostascint® scan, and/or PSA doubling time calculation was performed in 35 of the 37 patients experiencing biochemical progression, before administration of salvage radiation or hormonal therapy. Post-progression serum PSA doubling time was calculated for patients who had biochemical progression, and at least three PSA measurements were performed after the date of progression using the formula: DT=log(2)×T/[log(final PSA)−log(initial PSA)] (Schmid et al., 1993) where DT is the serum PSA doubling time, T is the time interval between the initial and final PSA level, final PSA is the pre-radiation PSA level, and initial PSA is the PSA level noted at the time of the post-operative biochemical progression. The natural logarithm was used in all logarithmic transformations. Nineteen (51%) of the 37 patients who had biochemical progression were treated at the Methodist Hospital with external beam radiation therapy limited to the prostatic fossa. Radiation was delivered with 15 to 20 MV photons, and the four-fields technique was used with customized field sizes. Total radiation therapy dose ranged from 60 to 66 Gy, delivered daily in fractions. A complete response to salvage radiation therapy was defined as the achievement and maintenance of an undetectable serum PSA level. Radiation therapy was considered to have failed if the post-radiation serum PSA levels did not fall to, and remain at, an undetectable level (Kattan et al., 2000; Leventis et al., 2001).

Statistical Analysis

Differences in TGF-β₁, IL-6 and IL6sR levels between clinical and pathologic features were tested by the Mann Whitney U-test. Spearman's rank correlation coefficient was used to compare ordinal and continuous variables. Logistic regression was used for multivariate analysis of binary outcome variables. Multivariable survival analysis was performed with the Cox proportional hazard regression model. Pre-operative PSA level had a skewed distribution and therefore was modeled with a log transformation. Clinical stage was evaluated as T1 versus T2 versus T3a. Biopsy and radical prostatectomy Gleason sum were evaluated as grade 2 to 6 versus grade 7 to 10. Differences in TGF-β₁, IL-6 and IL6sR levels between pre- and post-operative samples were tested by Wilcoxon signed-rank test. Statistical significance in this study was set as P<0.05. All reported P values are two-sided. All analyses were performed with SPSS statistical package (SPSS version 10.0 for Windows).

Results

Association of Pre- and Post-Operative Plasma Levels of TGF-β₁, IL-6 and IL6sR with Clinical and Pathologic Characteristics

Clinical and pathologic characteristics of the 302 consecutive prostatectomy patients and association with pre- and post-operative plasma TGF-β₁, IL-6 and IL6sR levels are shown in Table 13.

TABLE 13

| | | TGF-β₁ (ng/mL) | | | | IL-6 (pg/mL) | | | | IL-6sR (ng/mL) | | | |
|---|---|---|---|---|---|---|---|---|---|---|---|---|---|
| | | Pre-operative | | Post-operative | | Pre-operative | | Post-operative | | Pre-operative | | Post-operative | |
| | | Median | | Median | | Median | | Median | | Median | | Median | |
| | No. Pts (%) | (Range) | P* | (Range) | P* | (Range) | P* | (Range) | P* | (Range) | P* | (Range) | P* |
| Prostatectomy patients | 302 | 3.9 (1.0-19.8) | | 3.2 (0.5-18.1) | | 1.9 (0.0-8.0) | | 1.5 (0.0-7.3) | | 26.3 (10.4-48.2) | | 20.6 (7.9-46.1) | |
| Clinical stage | | | | | | | | | | | | | |
| T1 | 141 (47) | 3.8 (1.0-19.3) | .355 | 3.2 (1.0-18.1) | .909 | 1.9 (0.0-7.6) | .922 | 1.3 (0.0-7.7) | .171 | 24.7 (11.4-42.7) | .190 | 19.7 (7.9-45.0) | .135 |
| T2 | 151 (50) | 3.9 (1.0-19.8) | | 3.2 (0.5-13.9) | | 1.9 (0.0-8.0) | | 1.6 (0.0-6.3) | | 26.7 (10.4-48.2) | | 20.9 (8.8-46.1) | |
| T3a | 10 (3) | 4.1 (2.8-17.0) | | 3.4 (1.1-14.3) | | 1.4 (0.444) | | 1.4 (0.0-3.4) | | 24.8 (15.1-39.7) | | 21.5 (10.5-28.4) | |
| Biopsy Gleason sum | | | | | | | | | | | | | |
| 2-6 | 199 (66) | 3.7 (1.0-19.8) | .077 | 3.1 (0.6-18.1) | .104 | 1.8 (0.0-8.0) | .175 | 1.4 (0.0-7.7) | .251 | 25.3 (11.4-48.2) | .087 | 20.1 (7.9-46.1) | .075 |
| 7-10 | 103 (34) | 4.2 (1.0-17.3) | | 3.3 (0.5-14.3) | | 2.0 (0.0-6.6) | | 1.6 (0.0-5.6) | | 27.6 (10.4-45.9) | | 21.6 (8.8-45.0) | |
| RP extraprostatic extension only† | | | | | | | | | | | | | |
| Negative | 195 (65) | 3.4 (1.0-15.9) | .028 | 2.7 (0.5-18.1) | <.001 | 1.8 (0.0-8.0) | .066 | 1.5 (0.0-7.7) | .251 | 24.8 (10.4-45.9) | .076 | 19.6 (7.9-46.1) | .434 |
| Positive | 105 (35) | 4.3 (1.3-19.8) | | 3.8 (0.8-14.3) | | 2.1 (0.0-6.6) | | 1.5 (0.0-5.2) | | 27.0 (12.0-48.2) | | 21.3 (8.8-45.0) | |
| RP seminal vesicle involvement† | | | | | | | | | | | | | |
| Negative | 279 (93) | 3.7 (1.0-19.8) | .029 | 2.9 (0.5-18.1) | .023 | 1.9 (0.0-8.0) | .326 | 1.5 (0.0-7.7) | .434 | 25.5 (10.4-48.2) | .698 | 21.6 (7.9-46.1) | .427 |
| Positive | 21 (7) | 4.6 (1.7-17.0) | | 3.6 (1.2-14.3) | | 2.0 (0.4-4.0) | | 1.4 (0.9-3.6) | | 27.3 (11.7-41.6) | | 19.5 (8.8-45.0) | |
| RP surgical margin† | | | | | | | | | | | | | |
| Negative | 260 (87) | 3.9 (1.0-19.8) | .304 | 3.2 (0.5-18.1) | .756 | 1.9 (0.0-8.0) | .278 | 1.4 (0.0-6.3) | .987 | 26.0 (10.4-48.2) | .782 | 21.6 (7.9-46.1) | .202 |
| Positive | 40 (13) | 3.8 (1.3-7.9) | | 3.1 (0.8-5.2) | | 2.0 (0.0-6.6) | | 1.5 (0.0-7.7) | | 26.8 (11.7-43.8) | | 18.4 (8.8-38.2) | |
| RP Gleason sum† | | | | | | | | | | | | | |
| 2-6 | 147 (49) | 3.8 (1.0-19.3) | .912 | 3.0 (0.6-18.1) | .117 | 1.7 (0.0-8.0) | .014 | 1.4 (0.0-7.7) | .333 | 23.5 (11.4-45.4) | .034 | 20.7 (9.8-45.2) | .147 |
| 7-10 | 153 (51) | 3.9 (1.0-19.8) | | 3.4 (0.5-14.3) | | 2.1 (0.0-6.6) | | 1.6 (0.0-5.6) | | 28.6 (10.4-48.2) | | 20.6 (7.9-46.1) | |
| RP lymph node metastases | | | | | | | | | | | | | |
| Negative | 296 (98) | 3.8 (1.0-19.8) | <.001 | 3.0 (0.5-18.1) | <.001 | 1.8 (0.0-8.0) | .005 | 1.3 (0.0-7.7) | .084 | 24.4 (10.4-37.8) | <.001 | 19.3 (7.8-46.1) | .101 |
| Positive | 6 (2) | 7.1 (3.3-17.3) | | 6.5 (3.3-14.3) | | 2.6 (1.4-7.6) | | 1.6 (0.9-5.6) | | 29.8 (17.0-44.3) | | 21.0 (10.5-39.9) | |
| RP DNA ploidy‡ | | | | | | | | | | | | | |
| Diploid | 125 (49) | 3.6 (1.1-15.9) | .151 | 3.0 (0.8-18.1) | .543 | 1.9 (0.0-6.5) | .807 | 1.4 (0.0-5.2) | .288 | 26.0 (10.4-44.3) | .804 | 20.8 (11.4-46.1) | .643 |
| Aneuploid or tetraploid | 129 (51) | 4.0 (1.0-19.8) | | 3.3 (1.1-14.3) | | 1.9 (0.0-8.0) | | 1.6 (0.0-4.2) | | 26.6 (12.1-43.8) | | 19.5 (7.9-36.1) | |

| | TGF-β₁ | | | | IL-6 | | | | IL-6sR | | | |
|---|---|---|---|---|---|---|---|---|---|---|---|---|
| | Pre-operative | | Post-operative | | Pre-operative | | Post-operative | | Pre-operative | | Post-operative | |
| | CC§ | P | CC§ | P | CC§ | P | CC§ | P | CC§ | P | CC§ | P |
| Age | 0.024 | .616 | 0.025 | .679 | 0.042 | .379 | 0.080 | .239 | 0.022 | .650 | 0.091 | .181 |
| Pre-operative PSA | .469 | .004 | 0.055 | .358 | 0.177 | <.001 | 0.077 | .254 | 0.201 | .011 | 0.057 | .401 |
| RP tumor volume∥ | 0.109 | .095 | 0.112 | .159 | 0.172 | .018 | 0.068 | .454 | 0.198 | .016 | 0.046 | .610 |
| Pre-operative TGF-β₁ | — | — | 0.451 | <.001 | 0.116 | .019 | 0.091 | .069 | 0.193 | .038 | 0.088 | .207 |
| Post-operative TGF-β₁ | 0.451 | <.001 | — | — | 0.107 | .079 | 0.126 | .075 | 0.077 | .206 | 0.002 | .981 |
| Pre-operative IL-6 | 0.116 | .019 | 0.107 | .079 | — | — | 0.514 | <.001 | 0.443 | <.001 | .209 | .002 |
| Post-operative IL-6 | 0.091 | .069 | 0.126 | .075 | 0.514 | <.001 | — | — | 0.188 | .006 | 0.203 | .003 |
| Pre-operative IL-6sR | 0.193 | .038 | 0.077 | .206 | 0.443 | <.001 | 0.188 | .006 | — | — | 0.756 | <.001 |
| Post-operative IL-6sR | 0.088 | .207 | 0.002 | .981 | 0.209 | .002 | 0.203 | .003 | 0.756 | <.001 | — | — |

RP = Radical prostatectomy.
CC = Correlation coefficient
*Mann Whitney U test.
†RP extracapsular extension status, RP seminal vesicle involvement status, RP surgical margin status, and RP Gleason sum were not available for 2 patients, who did not undergo a prostatectomy because of positive pelvic lymph nodes at the time of surgery.
‡DNA ploidy was unavailable for 48 patients.
§Spearman's correlation coefficients.
∥Radical prostatectomy tumor volume was unavailable for 47 patients.

Pre-operative and post-operative plasma TGF-β₁ levels were elevated in patients with extraprostatic extension (P=0.028 and P<0.001, respectively), seminal vesicle involvement (P=0.029 and P=0.023, respectively), and regional lymph node metastases (P<0.001 and P<0.001, respectively). Preoperative IL-6 and IL6sR levels were elevated in patients with prostatectomy Gleason sum >7 (P=0.014 and P=0.034, respectively) and regional lymph node metastases (P=0.005 and P<0.001, respectively). The mean pre-operative PSA was 8.9±7.0 ng/mL (median 7.1, range 0.2 to 59.9). Pre-treatment TGF-β₁, IL-6, and IL6sR levels were positively correlated with pre-operative PSA levels (P=0.004, P<0.001, and P=0.011, respectively). Pre-treatment IL-6 and IL6sR levels were also positively correlated with prostatic tumor volume (P=0.018 and P=0.016, respectively). Post-operative IL-6 and IL6sR levels were not associated with any of the clinical or pathologic parameters.

In univariable logistic regression analyses, pre-operative TGF-β₁ levels predicted organ confined disease (P=0.017, Hazard ratio 0.902, 95% CI 0.828-0.982), but pre-operative IL-6 and IL6sR did not (P=0.118 and P=0.079, respectively). In a pre-operative multivariable model, clinical stage (P=0.035) and biopsy Gleason sum (P<0.001) were the only predictors of organ confined disease, when adjusted for the effects of pre-operative PSA (P=0.087), pre-operative TGF-β₁ (P=0.112), pre-operative IL-6 (P=0.639), and pre-operative IL6sR (P=0.725).

Association of Pre- and Post-Operative Plasma Levels of TGF-β₁, IL-6 and IL6sR with Prostate Cancer Progression

Overall, only 14% of patients (43 of 302) had cancer progression with a median post-operative follow-up of 50.7 months (range 1.2 to 73.5). The overall PSA progression-free survival was 88.8±1.5% (Standard error, SE) at 3 years and 85.1±1.9% (SE) at 5 years. On univariable Cox proportional hazards regression analyses (Table 14), pre- and post-operative TGF-β₁ (P<0.001), pre-operative IL-6 (P<0.001), pre-operative IL6sR (P<0.001), pre-operative PSA (P<0.001), biopsy and prostatectomy Gleason sum (P<0.001 and P<0.001, respectively), extraprostatic extension (P<0.001), seminal vesicle involvement (P<0.001), and surgical margin status (P<0.001) were associated with cancer progression, but post-operative IL-6 (P=0.162), post-operative IL6sR (P=0.079), and clinical stage (P=0.103) were not.

TABLE 14

| | Model 1 | | | Model 2 | | | Model 3 | | |
|---|---|---|---|---|---|---|---|---|---|
| | Hazard ratio | 95% CI | P | Hazard ratio | 95% CI | P | Hazard ratio | 95% CI | P |
| Pre-Operative PSA* | 1.323 | 0.872-2.009 | .183 | 1.291 | 1.128-2.446 | .174 | 1.577 | 0.977-2.546 | .062 |
| Extraprostatic extension | 1.085 | 0.581-2.027 | .798 | 0.974 | 0.487-1.948 | .941 | 1.046 | 0.432-1.765 | .706 |
| Seminal vesicle involvement | 2.212 | 1.138-4.699 | .020 | 1.202 | 0.562-2.571 | .235 | 1.269 | 0.572-2.816 | .258 |
| RP Gleason sum† | 4.281 | 1.838-9.975 | <.001 | 4.042 | 1.657-9.855 | <.001 | 3.706 | 1.494-9.191 | .005 |
| Surgical margin status | 2.595 | 1.232-4.276 | .009 | 1.453 | 0.772-2.734 | .107 | 1.501 | 0.784-2.874 | .114 |
| Pre-Operative IL-6 | 1.629 | 0.989-1.495 | .055 | — | — | — | 1.122 | 0.953-1.081 | .332 |
| Pre-Operative IL-6sR | 1.843 | 1.001-1.088 | .045 | — | — | — | 1.215 | 0.953-1.452 | .268 |
| Pre-Operative TGF-β₁ | 1.151 | 1.057-2.253 | <.001 | — | — | — | 1.058 | 0.870-1.285 | .574 |
| Post-Operative IL-6 | — | — | — | 1.154 | 0.923-1.443 | .208 | 1.031 | 0.790-1.346 | .822 |
| Post-Operative IL-6sR | — | — | — | 0.992 | 0.952-1.034 | .698 | 0.984 | 0.932-1.039 | .566 |
| Post-Operative TGF-β₁ | — | — | — | 2.305 | 1.188-3.532 | <.001 | 2.241 | 1.247-3.356 | .013 |

RP = radical prostatectomy
*Pre-operative PSA level had a skewed distribution and therefore was modeled with a log transformation.
†Radical prostatectomy Gleason sum was evaluated as grade 2 to 6 versus grade 7 to 10.

In a pre-operative multivariable model, pre-operative TGF-β₁ (P 0.010, Hazard ratio 1.710, 95% CI 1.078-2.470), IL6sR (P=0.038, Hazard ratio 1.515, 95% CI 1.011-2.061), and biopsy Gleason sum (P<0.001, Hazard ratio 2.896, 95% CI 1.630-5.145) were associated with cancer progression when adjusted for the effects of pre-operative PSA (P=0.058), pre-operative IL-6 (P=0.062), and clinical stage (P=0.837).

Pre- and post-operative TGF-β₁, IL-6 and IL6sR were analyzed in separate post-operative multivariable Cox proportional hazards regression analyses that also included extracapsular extension, seminal vesicle involvement, surgical margin status, pathologic Gleason sum, and pre-operative PSA. In the first model that included pre-operative levels of the candidate markers, pre-operative TGF-β₁ (P<0.001) and IL6sR (P=0.045) along with prostatectomy Gleason sum (P<0.001), seminal vesicle involvement (P=0.020), and surgical margin status (P=0.009) were associated with cancer progression. In the second model that included post-operative levels of the candidate markers, only post-operative TGF-β₁ (P<0.001) and prostatectomy Gleason sum (P<0.001) were associated with disease progression. In the third model that included pre- and post-operative levels of TGF-β₁, IL-6 and IL6sR, only post-operative TGF-β₁ (P=0.013) and prostatectomy Gleason sum (P=0.005) were associated with prostate cancer progression.

Association of Pre- and Post-Operative Plasma Levels of TGF-β₁, IL-6 and IL6sR with Features of Aggressive Prostate Cancer Progression

Nineteen patients were categorized as having features of non-aggressive prostate cancer progression because their PSA doubling times were equal or greater than 10 months (n=18; median 23, range 12-224) and/or because they achieved a complete response to local salvage radiation therapy (n=5). Twenty-four patients were categorized as having features of aggressive cancer progression because of positive lymph nodes found at the time of radical prostatectomy (n=6), of a positive metastatic work-up (bone or Prostascint® scan; n=4), because their PSA doubling times were less than 10 months (n=23; median 7, range 1-9), and/or because they failed to respond to local radiation therapy (n=14). Pre- and post-operative TGF-β₁ levels (P<0.001 and P<0.001, respectively), pre-operative IL-6 levels (P<0.001) and pre-operative IL6sR levels (P<0.001) were higher in patients with features of aggressive failure than in those with features of non-aggressive failure. In contrast, post-operative levels of IL-6 and IL6sR were not different between patients with features of aggressive failure and those with features of non-aggressive failure (P=0.062 and P=0.075, respectively). In a pre-operative multivariable Cox proportional hazards regression analysis, pre-operative plasma TGF-β₁ (P<0.001, Hazard ratio 1.298, 95% CI 1.093-1.716), pre-operative IL6sR (P=0.021, Hazard ratio 1.312, 95% CI 1.099-1.837), and biopsy Gleason sum (P=0.010, Hazard ratio 3.112, 95% CI 1.122-8.534) were associated with aggressive prostate cancer progression when adjusted for the effects pre-operative IL-6 (P 0.058), pre-operative PSA (P=0.086), and clinical stage (P=0.432).

Pre- and post-operative TGF-β₁, IL-6 and IL6sR were analyzed in separate post-operative multivariable Cox proportional hazards regression analyses that also included extracapsular extension, seminal vesicle involvement, surgical margin status, pathologic Gleason sum, and pre-operative PSA (Table 15). In the first model that included pre-operative levels of the candidate markers, pre-operative TGF-β₁ (P=0.013) and IL6sR (P=0.042) along with prostatectomy Gleason sum (P=0.009) and seminal vesicle involvement (P=0.027) were associated with aggressive cancer progression. In the second model that included post-operative levels of the candidate markers, only post-operative TGF-β₁ (P=0.012), seminal vesicle involvement (P=0.044), and prostatectomy Gleason sum (P=0.021) were associated with aggressive disease progression. In the third model that included pre- and post-operative levels of the candidate markers, only post-operative TGF-β₁ (P=0.043), prostatectomy Gleason sum (P=0.037), and seminal vesicle involvement (P=0.049) were associated with aggressive prostate cancer progression.

TABLE 16

| | | TGF-β₁ (ng/mL) | | | | IL-6 (pg/mL) | | | | IL-6sR (ng/mL) | | | |
|---|---|---|---|---|---|---|---|---|---|---|---|---|---|
| | | | | Percent | | | | Percent | | | | Percent | |
| | No. | Pre- | Post- | De- | | Pre- | Post- | De- | | Pre- | Post- | De- | |
| | Pts. | Operative | Operative | crease | P* | Operative | operative | crease | P* | Operative | Operative | crease | P* |
| All patients | 302 | 3.9 | 3.2 | 18% | .029 | 1.9 | 1.5 | 21% | <.001 | 26.3 | 20.6 | 22% | <.001 |
| | | (1.0-19.8) | (0.5-18.1) | | | (0.0-8.0) | (0.0-7.3) | | | (10.4-48.2) | (7.9-46.1) | | |
| Patients who | 43 | 4.7 | 4.3 | 9% | .074 | 2.3 | 1.6 | 30% | <.001 | 30.6 | 22.3 | 27% | <.001 |
| experienced | | (1.6-19.8) | (1.2-18.1) | | | (1.0-8.0) | (0.0-7.3) | | | (13.2-48.2) | (7.9-46.1) | | |
| cancer | | | | | | | | | | | | | |
| progression | | | | | | | | | | | | | |
| Patients | 259 | 3.6 | 2.4 | 33% | <.001 | 1.7 | 1.4 | 18% | .042 | 24.1 | 20.1 | 17% | .034 |
| who did not | | (1.0-10.3) | (0.5-8.3) | | | (0.0-7.1) | (0.0-5.8) | | | (10.4-32.3) | (7.9-33.4) | | |
| experience | | | | | | | | | | | | | |
| cancer | | | | | | | | | | | | | |
| progression | | | | | | | | | | | | | |

*Wilcoxon signed-rank test.

Pre-Versus Post-Prostatectomy TGF-β₁, IL-6 and IL6sR Levels

Overall, post-operative TGF-β₁, IL-6, and IL6sR levels were all lower than pre-operative levels (P=0.029, P=<0.001, and P<0.001, respectively; Table 16). In the subgroup of patients who experienced disease progression, post-operative IL-6 and IL6sR levels were both lower than pre-operative IL-6 and IL6sR levels (P<0.001 and P<0.001, respectively). However, post-operative TGF-β₁ levels were not different than pre-operative TGF-β₁ levels (P=0.074). In the subgroup of patients who did not experience cancer progression, pre-operative levels of TGF-β₁, IL-6, and IL6sR declined after surgery P<0.001, P=0.042, and P=0.034, respectively).

Discussion

The present study confirmed previously reported observations that pre-operative plasma levels of TGF-β₁, IL-6 and IL6sR are associated with established features of aggressive primary prostate cancer, with clinically evident and occult metastases present at the time of primary treatment, and with eventual disease progression (Shariat et al., 2001a; Shariat et al., 2001b). While all three of these markers were associated with frank metastatic disease to lymph nodes, definite distinctions were defined in the associations of these markers with other clinical and pathologic parameters of the local tumor. For example, pre-operative plasma levels of TGF-β₁ were associated with features of locally invasive disease, e.g., extraprostatic extension and seminal vesicle invasion, but not the histologic grade of disease. On the other hand, pre-operative plasma levels of IL-6 and IL6sR were associated with pathologic grade of disease (i.e., Gleason sum), but not extraprostatic extension or seminal vesicle invasion. Furthermore, pre-operative levels of IL-6 and IL6sR were positively correlated with local tumor volume, while TGF-β₁ levels were not.

Not surprisingly, therefore, plasma levels of all three markers decreased significantly after prostate removal when evaluated in all patients. This remained true for patients who did not experience cancer progression. Interestingly, while the decrease in TGF-β₁ levels was greater in patients who did not experience cancer progression compared to all patients (33% versus 18%), the decrease in IL-6 and IL6sR was proportionally less marked (18% versus 21% and 17% versus 22%, respectively). In contrast, in patients who experienced disease progression, the fall in post-operative IL-6 and IL6sR levels after prostate removal was significant (30% and 27%, respectively), while post-operative TGF-β₁ levels fell only minimally (9%) and were not significantly different from pre-operative TGF-β₁ levels. These findings are similar to findings reported for other surgically treated malignancies with TGF-β₁ decreasing only in patients apparently cured after definitive surgery and remaining elevated in patients found to have lymph node or distant metastases and/or residual disease after surgery (Kong et al., 1995; Kong et al., 1999; Tsushima et al., 2001). In addition, in concordance with the present findings, Tsushima et al. (2001) found that in patients undergoing colon resection for colorectal cancer, both the pre- and post-operative TGF-β₁ level were associated with development of liver metastases when controlling for the effects of age, pre- and post-operative carcinoembryonic antigen level, gender, and clinical tumor grade and stage. On the other hand, circulating levels of IL-6 have been reported to significantly decrease after surgery, regardless of whether cure was surgically achieved (Galizia et al., 2002).

Together, these data suggest that in patients with cancer, blood levels of IL-6 and IL6sR are produced primarily by tumor cells in the primary prostate cancer. Furthermore, circulating levels of IL-6 and its soluble receptor appear to be only associated with the potential of prostate cancer to metastasize, but not with the metastases themselves. In contrast, it appears that circulating levels of TGF-β₁ are more closely associated with the metastatic process, either due to direct release from foci of metastatic tumor or to the host's response to cancer invasion and dissemination. The increased predictive value of post-operative TGF-β₁ levels seen in post-operative multivariable analysis for the prediction of prostate cancer progression in the present cohort of patients supports this concept. While in a standard post-operative model that included pre-operative levels of the three candidate markers, both pre-operative IL6sR and TGF-β₁ were associated with prostate cancer progression, when only post-operative levels of three candidate markers were included in the model, post-operative TGF-β₁ was the sole candidate marker to be associated with cancer progression. Furthermore, when both pre- and post-operative levels of all three candidate markers were included in the same standard model, again only post-operative TGF-β₁ level remained associated with prostate cancer progression, once again demonstrating the loss of predictive value of IL-6 and IL6sR after removal of the primary tumor, but the improvement of predictive value of post-operative levels of TGF-β₁ over the pre-operative levels for prediction prostate cancer progression.

The present findings confirmed a previous study showing that that pre-operative IL6sR, but not pre-operative IL-6, was an independent predictor of cancer progression when modeled together in a standard pre-operative multivariable analysis (Shariat et al., 2001a). IL-6 acts through a hexametric cytokine receptor complex composed of an IL-6-specific receptor subunit and a signal transducer, gp130, that is also used by other cytokine receptors (Hirano, 1998). The binding of IL-6 to gp130 activates the Janus kinase/STAT3 signal transduction cascade, in which STAT factors translocate to the nucleus where they activate the transcription of target genes that play a critical role in cell survival, the G/S-phase cell cycle transition, cell movement, and cell differentiation (Hirano et al., 2000; Heinrich et al., 1998). While Hobiscb et al. (2000) have shown by immunohistochemistry that both IL-6 and IL-6 receptor are over-expressed in clinically localized prostate cancer, Giri et al. (2001) have recently demonstrated that in many prostate cancer cases there was either increased IL-6 or IL-6 receptor expression, suggesting two independent modes of inducing increased activation of the downstream signal transduction cascade. In addition, IL6sR, which arises by proteolytic cleavage (Mullberg et al., 1994) or alternate splicing (Oh et al., 1996) of the cell surface IL-6 receptor, in addition to acting synergistically with IL-6 has been shown to be a potent regulator of IL-6 response in cells lacking IL-6 cell surface receptor expression (Tamura et al., 1993; Peters et al., 1998). For example, the presence of IL6sR has been shown to be necessary for IL-6 to activate Stat signaling cascade in prostatic intraepithelial neoplasia cells lacking membrane-bound IL-6 receptor (Liu et al., 2002). The stronger predictive value of pre-operative IL6sR over that of IL-6 for prostate cancer progression supports the role of IL6sR as an agonistic regulator of IL-6 functions, and suggests an underlying biological mechanism for its superiority to IL-6 for prognostic purposes in patients with prostate cancer.

Interestingly, the surgical margin status was associated with overall but not aggressive prostate cancer progression. Features of aggressive prostate cancer progression included either a positive metastatic work up or surrogate end points suggestive of the presence of metastasis or rapid progression to clinical metastatic disease (i.e., PSA doubling times of less than 10 months (Leventis et al., 2001; Pound et al., 1999; Roberts et al., 2001) and the failure to respond to salvage local radiation therapy (Kattan et al., 2000; Leventis et al., 2001). The other predictors of overall progression (seminal vesicle involvement, pathologic Gleason sum, pre-operative IL6sR and TGF-β₁) retained their predictive value for aggressive prostate cancer progression. These data support the notion that while seminal vesicle involvement, pathologic Gleason sum, pre-operative IL6sR and TGF-β₁ levels are associated with either established or occult metastatic disease, or the propensity to develop metastases, positive surgical margins are associated with local recurrence that is typically non-aggressive. In concordance with these findings, Epstein et al. (1996) reported that the surgical margin status is a strong predictor of local recurrence after radical prostatectomy. These data support the concept that positive surgical margins correlated with residual local tumor in the surgical bed, and are the result of incomplete resection of the prostate by the surgeon.

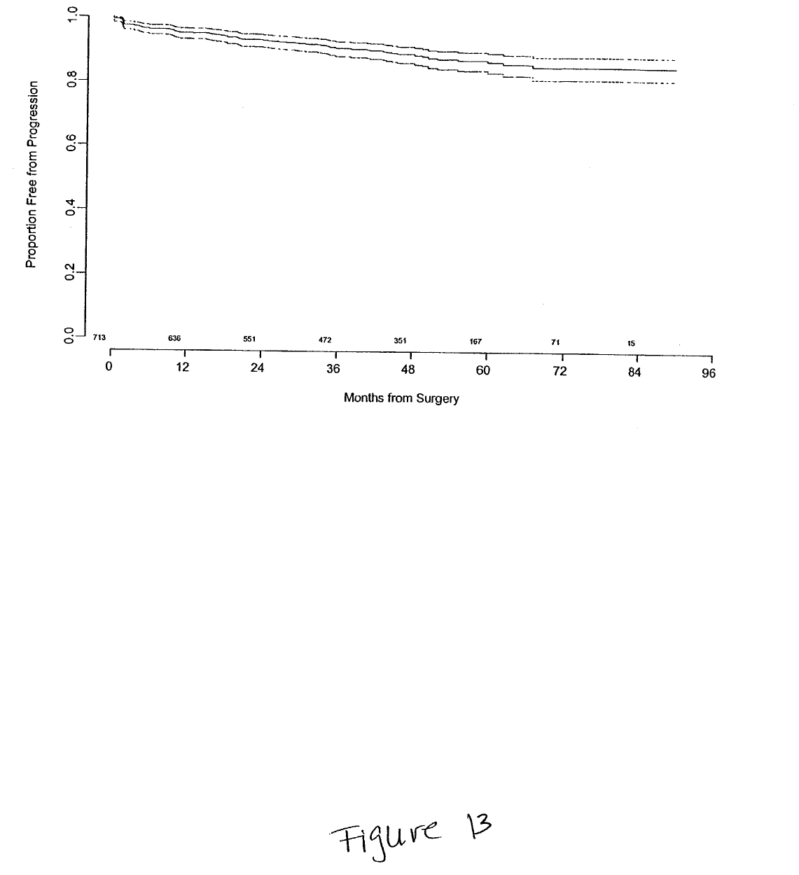

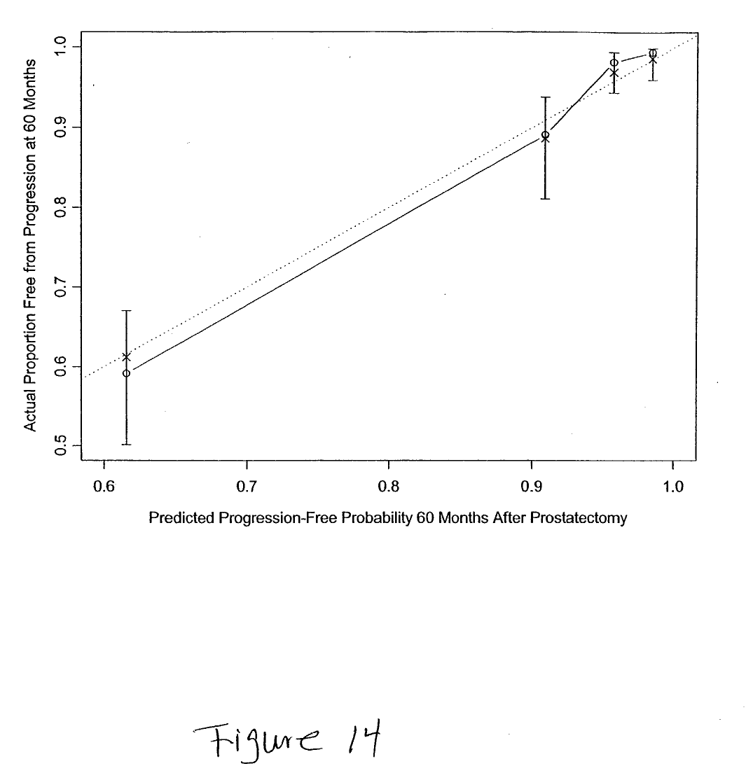

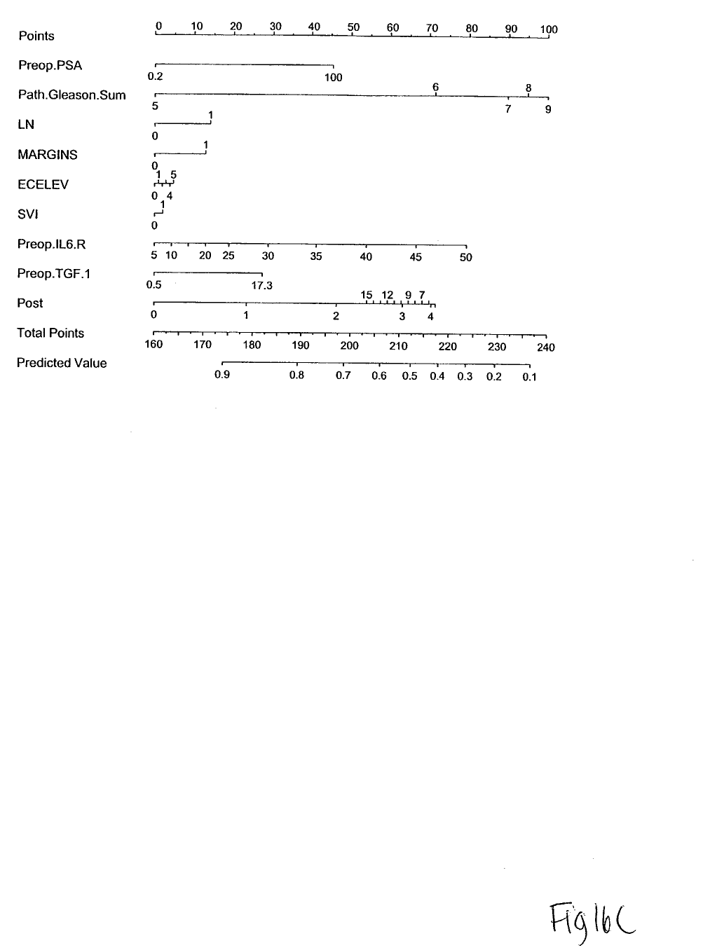

In conclusion, the present findings support the inclusion of pre-operative levels of TGF-β₁ and IL6sR to the standard pre-operative nomogram for prediction of recurrence after radical prostatectomy (see Example 5 and FIG. 12). The generalizability of these findings to other cancers suggests that the present observations and recommendations may be widely applicable to a variety of other cancers and cancer therapy modalities (i.e., radio- or chemo-therapy). Furthermore, early post-operative TGF-β₁ is a strong predictor of prostate cancer progression and is an excellent candidate marker for inclusion in other standard predictive models for progression after primary therapy for prostate cancer (FIGS. 16A-C).

### EXAMPLE 5

In patients undergoing radical prostatectomy for clinically localized disease, pre-operative plasma TGF-β₁, and IL6sR were associated with eventual prostate cancer progression, following adjustment for the effects of clinical stage, biopsy Gleason sum, and pre-operative PSA. Furthermore, pre-operative plasma levels of these markers were associated with aggressive disease progression, suggesting that this association was due to the presence of occult micrometastases already present at the time of surgery. As described below, TGF-β₁ and IL6sR were used with other markers of prostate disease, to prepare a nomogram.

Materials and Methods

Patients

All 814 patients admitted to The Methodist Hospital with the intent to treat their clinically localized prostate cancer (cT1c-3a, NX MO) with radical retropubic prostatectomy by full-time faculty were potential candidates for this analysis. Serum, plasma, and consent were obtained for 800 of these men. Each patient was assigned a clinical stage according to the 1992 TNM (i.e., tumor-node-metastasis) classification system (T1, nonpalpable tumor confined to the prostate; T2, confined tumor palpable or visible by imaging; T3a, palpable or visible tumor extending through the capsule of the prostate unilaterally; NX, regional nodal metastases not assessed clinically; MO, no evidence of distant metastases). Pelvic lymph node dissections were performed on all men. Radical prostatectomy was aborted in 2 of the 17 patients who were found to have nodal metastases on frozen section analysis during the operation; these men are not excluded from the analysis. However, 26 men initially treated with definitive radiotherapy (23 external beam radiation therapy and 3 cryotherapy) and 56 who were treated with neoadjuvant hormonal therapy before the radical procedure were excluded from the analysis. The five patients with one or more of the following missing values were excluded (PSA, N=1; Biopsy Gleason Grade, N=3; Clinical Stage, N=1; Disease Follow-up Status, N=1). This left 713 men for analysis.

The median age of all patients was 62 years (range, 40-81 years), and 86% of the patients were Caucasian. Pre-treatment PSA was measured by the Hybritech Tandem-R assay (Hybritech, Inc., San Diego, Calif.). The Gleason grade of each tumor was assigned by a single pathologist. Percent of cores positive was calculated by taking the ratio of the positive cores to the total cores removed, and multiplying by 100. IL6sR and TGF-β₁ were measured as described previously (Examples 1-2). Serum and plasma samples were collected after a pre-operative overnight fast on the morning of the day of surgery, at least 4 weeks after transrectal-guided needle biopsy of the prostate. Blood was collected into Vacutainer CPT 8-mL tubes containing 0.1 mL of 1 M sodium citrate (Becton Dickinson Vacutainer Systems, Franklin Lakes, N.J.) and centrifuged at room temperature for 20 minutes at 1500×g. The top layer corresponding to plasma was decanted using sterile transfer pipettes and immediately frozen and stored at −80° C. in polypropylene cryopreservation vials (Nalgene, Nalge Nunc, Rochester, N.Y.). For quantitative measurements of IL6sR and TGF-β₁ levels, quantitative immunoassays (R&D Systems, Minneapolis, Minn.) were used. For TGF-β₁, prior to assessment, an additional centrifugation step of the plasma was performed at 10,000×g for 10 minutes at room temperature for complete platelet removal. Recombinant TGF-β₁ was used as standard. Every sample was run in duplicate, and the mean was used for data analysis. The differences between the two measurements were minimal. The clinical characteristics appear in Table 17.

TABLE 17

| | No. of | |
|---|---|---|
| | Patients | % |
| Clinical Stage | | |
| T1c | 318 | 44.6 |
| T2a | 175 | 24.5 |
| T2b | 117 | 16.4 |
| T2c | 72 | 10.1 |
| T3a | 31 | 4.3 |
| Primary Biopsy Gleason Grade | | |
| 1 | 1 | 0.1 |
| 2 | 77 | 10.8 |
| 3 | 540 | 75.7 |
| 4 | 94 | 13.2 |
| 5 | 1 | 0.1 |
| Secondary Biopsy Gleason Grade | | |
| 1 | 3 | 0.4 |
| 2 | 50 | 7.0 |
| 3 | 476 | 66.8 |
| 4 | 178 | 25.0 |
| 5 | 6 | 0.8 |

| | |
|---|---|
| PSA | |
| Minimum | 0.2 |
| 1st quartile | 4.9 |
| Median | 6.8 |
| Mean | 8.5 |
| 3rd quartile | 9.8 |
| Maximum | 100.0 |
| Percent of Cores Positive | |
| Minimum | 7.14 |
| 1st quartile | 16.67 |
| Median | 33.33 |
| Mean | 36.99 |
| 3rd quartile | 50.00 |
| Maximum | 100.00 |
| Pre-Operative IL6sR | |
| Minimum | 5.88 |
| 1^{st} quartile | 21.30 |
| Median: | 25.70 |
| Mean: | 25.87 |
| 3^{rd} quartile | 29.60 |
| Maximum | 48.15 |
| Pre-Operative TGF-β₁ | |
| Minimum | 0.50 |
| 1^{st} quartile | 2.84 |
| Median | 3.72 |
| Mean | 3.92 |
| 3^{rd} quartile | 4.74 |
| Maximum | 17.30 |

Treatment Failure

The time of treatment failure was defined as the earliest date that the post-operative serum PSA level rose to 0.2 ng/mL. No patients were treated with hormonal therapy after surgery but before documented recurrence. Adjuvant radiation therapy was not considered failure. Patients whose radical prostatectomy was aborted due to metastatic disease in one or more lymph nodes were considered treatment failures from the day after surgery.

Statistical Analysis

Estimates of the probability of remaining free from recurrence were calculated using the Kaplan-Meier method. Multivariable analysis was conducted with Cox proportional hazards regression, which was the basis for the nomogram. The proportional hazards assumption was verified by tests of correlations with time and examination of residual plots. PSA and TGF-β₁ had skewed distributions and were log transformed. All non-nominal variables were fit with restricted cubic splines to allow potential nonlinear effects.

For nomogram validation, both discrimination and calibration capabilities were assessed. Discrimination refers to the ability of the nomogram to rank patients by their risk, such that patients with higher risk of failure should be more likely to fail. Discrimination was assessed because it is easily quantifiable using the concordance index, which is similar to an area under the receiver operating characteristic curve, but for time-until-event data. The calibration of the nomogram was measured through visual examination of plots of predicted vs. actual probabilities. Bootstrapping was utilized to obtain more generalizable estimates of expected future performance. All statistical analyses were performed using S-Plus software (PC Version 2000 Professional, Redmond Wash.) with additional functions (called Design) added. All P values resulted from use of two-sided statistical tests.

Results

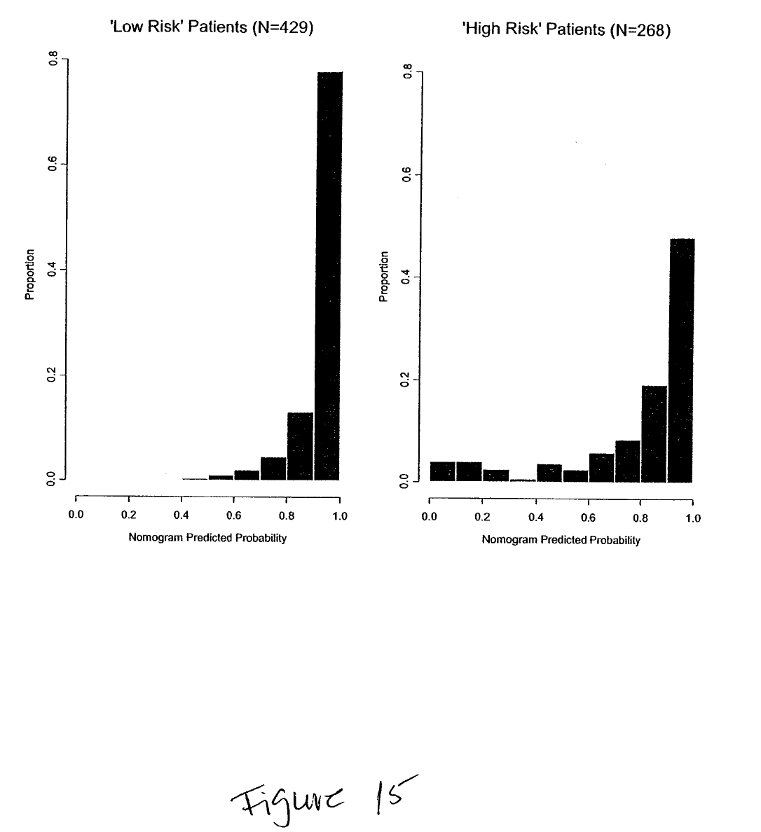

Of the 713 patients available for analysis, 79 had evidence of treatment failure following radical prostatectomy. For patients without disease recurrence, median follow-up was 49 months (range, 0.3 to 89.5 months), and 28% had their disease status verified within one year of this analysis. There were 166 patients with at least 60 months disease-free follow-up. Overall recurrence-free probability was 86% (95% CI=83%-89%) at 5 years (FIG. 13). In the multivariable Cox model, PSA (P=0.001), IL6sR (P<0.001), TGF-β₁ (P<0.001), primary Gleason grade (P=0.016), and secondary Gleason grade (P=0.037) were associated with PSA recurrence, while clinical stage (P=0.766) was not.

A nomogram was constructed based on the Cox model and appears in FIG. 12. The nomogram is used by first locating a patient's position on each predictor variable scale (PSA through TGFβ₁). Each scale position has corresponding prognostic points (top axis). For example, a PSA of 10 contributes approximately 21 points; this is determined by comparing the location of the 10 value on the “PSA” axis to the “Points” scale above and drawing a vertical line between the 2 axes. The point values for all clinical predictor variables are determined in a similar manner and are summed to arrive at a Total Points value. This value is plotted on the Total Points axis (second from the bottom). A vertical line drawn from the Total Points axis straight down to the 60 month PSA Progression-Free Probability axis will indicate the patient's probability of remaining free from cancer recurrence for 5 years assuming he remains alive.

The nomogram was evaluated for its ability to discriminate among patients' risk of recurrence. This was measured as the area under the receiver operating characteristic curve for censored data. This area represents the probability that, when two patients are randomly selected, one with recurrence and one with longer follow-up, the patient who failed first had the worse prognosis (from the nomogram). This measure can range from 0.5 (no better than chance) to 1.0 (perfect ability to discriminate). To derive an estimate of expected performance of the nomogram against new patients, bootstrapping was performed, a statistical method in which sampling, nomogram building, and nomogram evaluation are repeated a large number of times. With the use of bootstrapping, the area under the receiver operating characteristic curve was estimated to be 0.84. For comparison purposes, a model which omitted IL6sR and TGF-β₁ was bootstrapped and this model had a concordance index of 0.75.

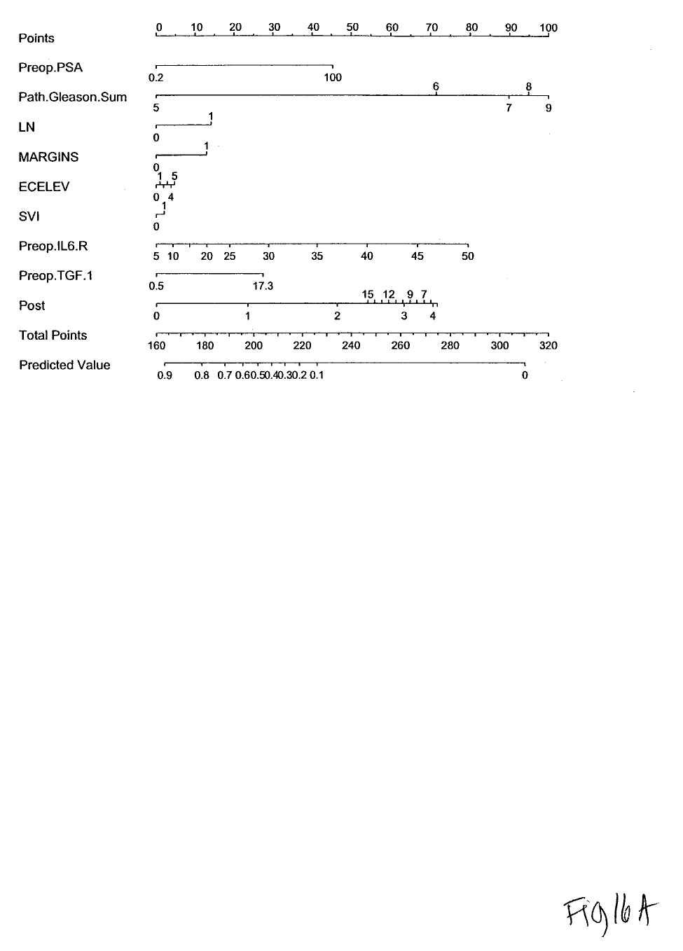

FIG. 14 illustrates how the predictions from the nomogram compare with actual outcomes for the 713 patients. The x-axis is the prediction calculated with use of the nomogram, and the y-axis is the actual freedom from cancer recurrence for patients. The dashed line represents the performance of an ideal nomogram, in which predicted outcome perfectly corresponds with actual outcome. The performance of the nomogram described herein is plotted as the solid line that connects the dots, corresponding to sub-cohorts (based on predicted risk) within the dataset. Note that, because the circles are relatively close to the dashed line, the predictions calculated with use of this nomogram approximate the actual outcomes. The X's indicate bootstrap-corrected estimates of the predicted freedom from disease recurrence, which are more appropriate estimates of expected accuracy. Most of the X's are close to the circles, indicating that the predictions based on use of the nomogram and modeled data (circles) are near that expected from use of the new data (the X's). The vertical bars in FIG. 14 indicate 95% confidence intervals based on the bootstrap analysis. In general, the performance of the nomogram appears to be within 9% of actual outcome, and possibly slightly more accurate at very high levels of predicted probability.

Percent of cores positive was missing in 35 of the 713 patients. When the subset of 678 patients who had values for this variable were examined, it was demonstrated that percent of cores positive was not associated with PSA recurrence when added to the Cox model (P=0.095). Although this finding alone would not be reason to exclude percent of cores positive from the final model and the nomogram, the model including percent of cores positive as a predictor had a concordance index lower than that of the reduced model which excluded percent of cores positive (0.83 vs. 0.84, both bootstrap corrected). This was apparently due to the reduced sample size associated with the model which contained percent of cores positive. Therefore, the model excludes percent of cores positive as a predictor.

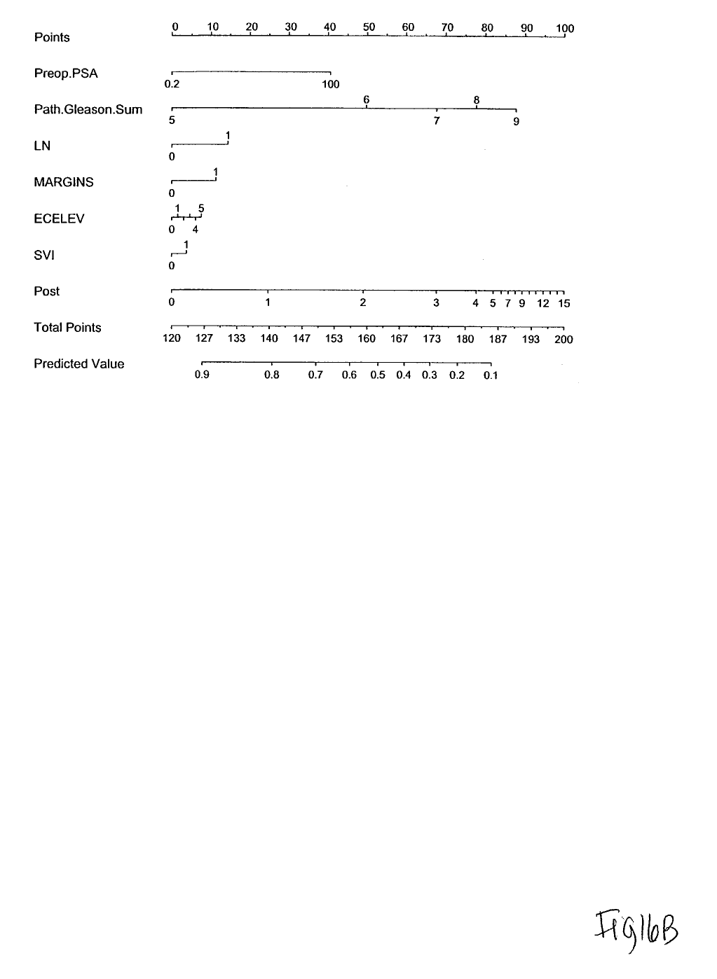

FIG. 15 compares the predictions of the nomogram described herein with those obtained by risk group analysis. For this figure, whether each patient was at “low” or “high” risk using a recently published risk stratification method was determined. FIG. 15 provides histograms of the nomogram predicted probabilities for patients within each risk group.

Discussion

A prognostic nomogram that adds two novel molecular markers, IL-6 soluble receptor and TGF-β₁, to a core group of clinical variables was constructed. This nomogram better predicts the risk of disease progression five years after radical prostatectomy for clinically localized prostate cancer. The addition of these two predictors resulted in a substantial improvement in discriminatory ability, increasing the bootstrap-corrected concordance index from 0.75 to 0.84.

IL6sR and TGF-β₁ were chosen because of their robust, distinctive, and complementary association with features of prostate cancer aggressiveness and metastases at the earliest disease stages prior to more obvious clinical evidence of metastases. A comprehensive evaluation of the performance of a host of potential biomarkers for prostate cancer invasion, progression, and metastasis including insulin-like growth factor-I and its binding proteins type 2 and 3, vascular endothelial growth factor and soluble vascular cell adhesion marker type 1, and interleuklin-6 was performed.

To further test the association of IL6sR and TGF-β₁ with prostate cancer, pre- and post-operative levels of TGF-β₁ and IL6sR in a consecutive cohort of 302 patients who underwent radical prostatectomy were measured. A strong association of pre-operative plasma levels of TGF-β₁ and IL6sR with established features of aggressive primary prostate cancer, with clinically evident and occult metastases present at the time of primary treatment, and with eventual disease progression was confirmed. While both of these markers were associated with frank metastatic disease to lymph nodes, definite distinctions in the associations of these markers with other clinical and pathologic parameters of the local tumor were identified. For example, pre-operative plasma levels of TGF-β₁ were associated with features of locally invasive disease, e.g., extraprostatic extension and seminal vesicle invasion, but not the histologic grade of disease. On the other hand, pre-operative plasma levels of IL6sR were associated with pathologic grade of disease (i.e., Gleason sum), but not extraprostatic extension or seminal vesicle invasion. Furthermore, pre-operative levels of IL6sR were positively correlated with local tumor volume, while TGF-β₁ levels were not. Furthermore, in patients who experienced disease progression, the post-operative TGF-β₁ levels fell only minimally (9%) and were not significantly different from pre-operative TGF-β₁ levels. On the other hand, after prostate removal, plasma IL6sR levels fell significantly both in patients who experienced disease progression and in those who did not. In the aggregate, these data suggest that circulating levels of IL-6 and its soluble receptor appear to be associated with the potential of prostate cancer to metastasize, but not with the metastases themselves. In contrast, it appears that circulating levels of TGF-β₁ are more closely associated with the metastatic process, either due to direct release from foci of metastatic tumor or to the host's response to cancer invasion and dissemination.

Others have demonstrated the value of using predictive parameters to stratify patients with regard to their risk of failure after primary therapy for prostate cancer. These approaches have primarily focused on using clinical parameters, e.g., pre-treatment PSA level or biopsy Gleason sum, to categorize patients into “low”, “intermediate”, and “high” risk groups. While superficially this approach may appear less cumbersome, the reduction of continuous risk variables, maintained in nomograms, into defined risk categories diminishes the level of predictive accuracy substantially. For example, using data from the patient cohort, classifying patients as low or high risk results in a concordance index of only 0.73, considerably less discriminating than the nomogram's concordance index of 0.84. In clinical terms, this reduction in the concordance index translates into profoundly different anticipated outcomes for patients faced with this disease. For example, FIG. 15 compares the predictions of the two approaches by plotting the nomogram prediction for patients categorized into previously published high and low risk groups. Note that most of the patients in the “high risk” group actually have very favorable and variable predictions from the nomogram. Informing a prostate cancer patient that he is at “high risk” is less useful than providing him with the best estimate of his predicted probability of remaining free from recurrence after choosing a mode of therapy. While neither prediction method can be considered a gold standard, the nomogram described herein appears to discriminate better and produce predictions which differ from a risk group approach by a clinically important degree.

The concordance index, based on standard clinical factors alone, was 0.75. This finding is consistent with earlier work, with nomograms for surgery, external beam radiation therapy, and brachytherapy, such that standard clinical factors alone cannot seem to achieve concordance indices above about 0.75. The addition of molecular markers appears to have affected a quantum increase in predictive accuracy, allowing for a concordance index of 0.84.

Improving the ability to predict treatment outcomes for clinically localized prostate cancer is critical. In this disease, treatment choices need to be tailored to the preferences of the individual patient who is forced to make a decision based on predictions of treatment outcomes. The risks of complications must be weighed against the risks of progression for untreated cancer and the predicted ability of aggressive therapy to delay or prevent progression. Partin and colleagues were among the first to provide a nomogram for use in this context by predicting final pathologic stage. This work has been extended to predicting PSA recurrence, an endpoint more definitive than final pathologic stage. Although treatment decision making is substantially more complicated than choosing the therapeutic strategy which appears to minimize the likelihood of disease recurrence, prediction of PSA recurrence is a valuable component of decision making for this disease.

In addition to serving as a prognostic tool, the nomogram in FIG. 14 is useful for interpreting the underlying Cox model. However, some assignments appear counter-intuitive (e.g., T2b>T2c), but these differences reflect variations in coefficient estimates and are not always statistically significant (two-sided P>0.05). Furthermore, it is important to consider possible changes in other variables (e.g., IL6sR) when comparing points across levels of a single variable (e.g., clinical stage). In other words, moving a patient along one axis likely moves him on other axes as well.

The nomogram was developed in a population of patients treated with radical prostatectomy, e.g., it is useful for patients who otherwise appear to be candidates for surgery, not necessarily all patients diagnosed with prostate cancer. Moreover, the nomogram predicts PSA recurrence as an endpoint. All patients who fail biochemically do not die of their disease or even progress to metastasis. Biochemical recurrence is an early warning sign that treatment has not necessarily been effective. No patient would select, nor would any clinician recommend, an aggressive therapy which is destined to lead to biochemical recurrence (i.e., 100% chance of failing biochemically) despite the loose association with metastasis and further disease sequelae. Furthermore, patients who fail biochemically, despite having no disease-related symptoms, have reduced quality of life.

In conclusion, a nomogram was developed that allows one to predict the probability of cancer recurrence after radical prostatectomy for localized prostate cancer (clinical stage T1 c-T3a NX MO) from the clinical stage, Gleason grade, serum PSA level, and plasma levels of IL6sR and of TGF-β₁. The nomogram may assist the physician and patient in deciding whether radical prostatectomy is an acceptable treatment option. It may also be useful in identifying patients at high risk of disease recurrence who may benefit from neoadjuvant treatment protocols. Furthermore, the incorporation of these molecular markers may improve prognostic tools for other prostate cancer treatment modalities as well.

### EXAMPLE 6

Subjects and Methods

Patient Population

All studies were undertaken with the approval and institutional oversight of the Institutional Review Board for the Protection of Human Subjects at Baylor College of Medicine. All 301 patients admitted to The Methodist Hospital with the intent to treat their clinically localized prostate cancer (cT1c-3a, NX, MO) with radical prostatectomy by surgeons of the Scott Department of Urology were potential candidates for this analysis. The clinical stage was assigned by the operative surgeon according to the 1992 TNM system. After obtaining consent, pre- and post-operative plasma specimens were obtained for 252 of these men. Sixteen men initially treated with hormonal therapy, five who were treated with definitive radiotherapy, and one who was treated with cryotherapy before surgery, were excluded from the analysis. No disease follow-up information was available for 15 men, and they were also excluded. This left 215 men for analysis. The mean patient age in this study was 61.8±7.3 y (median 62.6, range 40 to 80). Serum prostate specific antigen was measured by the Hybritech®Tandem-R assay (Hybritech, Inc., San Diego, Calif.).

Plasma VEGF and sVCAM-1 levels were also assessed in 40 healthy patients without cancer. This group included 2 sets of consecutive patients who participated in the prostate cancer screening program. They had no history of cancer or chronic disease, normal digital rectal examination and prostate specific antigen (PSA) less than 2 ng/mL. This PSA range is associated with an estimated probability of prostate cancer detection of less than 1% in the first 4 years after screening (Smith et al., 1996).

VEGF and sVCAM-1 Measurements

Plasma samples were collected after a pre-operative overnight fast on the morning of the day of surgery, at least 4 weeks after transrectal guided needle biopsy of the prostate. Blood was collected into Vacutainer®CPT™ 8 mL tubes containing 0.1 mL of Molar sodium citrate (Becton Dickinson Vacutainer Systems, Franklin Lakes, N.J.) and centrifuged at room temperature for 20 minutes at 1500×g. The top layer corresponding to plasma was decanted using sterile transfer pipettes. The plasma was immediately frozen and stored at −80° C. in polypropylene cryopreservation vials (Nalgene, Nalge Nunc, Rochester, N.Y.). It has been previously found that VEGF levels were higher when measured in serum than when measured in plasma. Since VEGF is present in platelet granules and is released upon platelet activation, the higher levels of VEGF in serum were likely due at least in part to release from damaged platelets, making the quantification of non-platelet derived VEGF less accurate (Spence et al., 2002). Therefore, for VEGF, prior to assessment, an additional centrifugation step of the plasma was performed at 10,000×g for 10 minutes at room temperature for complete platelet removal (Adams et al., 2000). For quantitative measurements of VEGF and sVCAM-1 levels, quantitative immunoassays were employed (R&D Systems, Minneapolis, Minn.). Every sample was run in duplicate, and the mean was used. Differences between the two measurements for both VEGF and sVCAM-I were minimal (intra-assay precision coefficients of variation: 8.49±11.10% and 4.86±6.31%, respectively).

Pathological Examination

All prostatectomy specimens were examined pathologically by a single pathologist, who was blinded to clinical outcome. The radical prostatectomy specimens were processed by whole-mount technique, and pathological parameters were evaluated in a manner previously described (Wheeler et al., 1994). Total tumor volume was computed by computerized planimetry from the whole-mount sections for 184 of the 215 prostatectomy patients.(Ohori et al., 1993).

Post-Operative Follow-Up

Patients generally were scheduled to have a digital rectal examination and serum PSA evaluation post-operatively every 3 months for the first year, semiannually from the second through the fifth year, and annually thereafter. Biochemical progression was defined as a sustained elevation, on 2 or more occasions, of PSA>0.2 ng/mL and was assigned to the date of the first value >0.2 ng/mL. Pelvic lymph node dissections were performed on all men. Radical prostatectomy was aborted in two of the eleven patients who were found to have nodal metastases on frozen section analysis during the operation; these men are not excluded from the analysis. The two patients with metastases to regional lymph nodes who had their prostates not removed were categorized among those with progression from the day after surgery. Six patients (3%) received adjuvant radiation therapy before biochemical progression because of positive surgical margins. Three of them subsequently experienced PSA relapse and was considered to have disease progression from the date of the first value >0.2 ng/mL, while the other three were censored on the date of the last follow-up examination. Of 215 patients who underwent radical prostatectomy, 42 had progression of disease.

Statistical Analysis

Differences in plasma VEGF and sVCAM-1 levels between clinical and pathologic features were tested by the Mann Whitney U-test and the Kiruskal Wallis test. Spearman's rank correlation coefficient was used to compare ordinal and continuous variables. Logistic regression was used for multivariable analysis of binary outcome variables. Multivariable survival analysis was performed with Cox proportional hazard regression model. Pre-operative PSA level had a skewed distribution and therefore was modeled with a log transformation. Biopsy and radical prostatectomy Gleason sum were evaluated as grade 2 to 6 versus grade 7 to 10. Statistical significance in this study was set as P<0.05. All reported P values are two-sided. All analyses were performed with SPSS statistical package version 111 for Windows (SPSS, Chicago, Ill.).

Results

Plasma VEGF and sVCAM-1 in Patients with Prostate Cancer Metastases

Plasma VEGF and sVCAM-1 levels were assessed in nine patients with bone scan-proven, metastatic prostate cancer, and 215 patients diagnosed with clinically localized prostate cancer. Neither of these patients were treated with either hormonal or radiation therapy before plasma collection. Plasma VEGF and sVCAM-1 levels in patients with prostate cancer metastatic to bones (median 31.3, range 15.3-227.1 and median 648.7, range 524.8-1907.1, respectively) were higher than those in patients with clinically localized disease (median 9.9, range 2.0-166.9 and median 581.8, range 99.0-2068.3, respectively; P values <0.001). Plasma levels for healthy controls were within the normal range reported by the ELISA company for both VEGF and sVCAM-1 (median 2.24, range 1.6 to 3.0 and median 555.0, range 398.0 to 712.0, P values <0.001 respectively)

Association of Pre-Operative Plasma VEGF and sVCAM-1 with Clinical and Pathologic Characteristics of Prostate Cancer

Clinical and pathologic characteristics of 215 prostatectomy patients and association with pre-operative plasma VEGF and sVCAM-1 levels are shown in Table 18. Pre-operative VEGF and sVCAM-1 levels were both elevated in patients with lymph node involvement (P<0.001 and P=0.012, respectively). However only pre-operative plasma VEGF was elevated in patients with biopsy and final Gleason sum ≧7 (P=0.036 and P=0.040, respectively) and extraprostatic extension (P=0.047). The mean pre-operative PSA was 9.15±1.01 ng/mL (median 7.3, range 1.1 to 60.1). Sixty-two patients (28%) had PSA levels of 10 ng/mL and beyond. On univariate logistic regression analyses pre-operative plasma VEGF levels were associated with organ-confined disease (Hazard ratio 0.991, 95% CI 0.983-0.998, P=0.016) and lymph node involvement (Hazard ratio 1.033, 95% CI 1.019-1.047, P<0.001), whereas pre-operative plasma sVCAM-1 levels were not (P=0.367 and P=0.063, respectively). On multivariate logistic regression analyses (Table 19), pre-operative plasma VEGF was associated with prostate cancer involvement of the lymph nodes (P<0.001) but not with confinement of the cancer to the prostate (P=0.528), when adjusted for the effects of standard pre-operative features and pre-operative plasma sVCAM-1.

TABLE 18

| | | | | | Pre-operative | | |
|---|---|---|---|---|---|---|---|
| | | Pre-operative VEGF (pg/mL) | | | sVCAM-1 (ng/mL) | | |
| | No. Pts (%) | Median | Range | P | Median | Range | P |
| Healthy Controls | 40 | 2.2 | 1.6-3.0 | | 555.0 | 328.0-712.0 | |
| Prostatectomy patients | 215 | 9.9 | 2.0-166.9 | <.001 | 581.8 | 116.0-2068.3 | .290 |
| Clinical stage | | | | | | | |
| T1c | 97 (45) | 9.3 | 4.1-166.9 | | 493.8 | 116.0-2068.3 | |
| T2a | 56 (26) | 9.6 | 4.1-153.4 | | 481.7 | 178.0-1807.6 | |
| T2b | 36 (17) | 12.2 | 2.0-151.8 | | 542.8 | 203.3-1144.9 | |
| T2c | 23 (11) | 14.1 | 4.5-97.4 | | 403.7 | 99.4-1201.1 | |
| T3a | 3 (1) | 34.1 | 9.9-134.4 | .054 | 345.40 | 314.3-888.7 | .203 |
| Biopsy Gleason sum | | | | | | | |
| 2-6 | 143 (67) | 9.6 | 2.0-166.9 | | 477.80 | 402.1-1807.6 | |
| 7-10 | 72 (33) | 13.2 | 4.8-153.4 | .036 | 531.05 | 116.0-2068.3 | .311 |
| RP extraprostatic | | | | | | | |
| extension only‡ | | | | | | | |
| Negative | 139 (65) | 9.6 | 2.0-166.9 | | 475.90 | 402.1-1807.6 | |
| Positive | 74 (35) | 12.4 | 4.4-151.8 | .047 | 524.20 | 99.4-2068.3 | .234 |
| RP seminal | | | | | | | |
| vesicle involvement‡ | | | | | | | |
| Negative | 198 (93) | 9.9 | 2.0-166.9 | | 490.90 | 402.1-2068.3 | |
| Positive | 15 (7) | 12.1 | 4.4-134.32 | .438 | 501.40 | 214.4-888.7 | .842 |
| RP surgical margin‡ | | | | | | | |
| Negative | 180 (85) | 9.6 | 2.0-166.9 | | 482.60 | 402.1-1807.6 | |
| Positive | 33 (15) | 12.1 | 4.8-125.1 | .116 | 515.00 | 99.4-2068.3 | .501 |
| RP Gleason sum‡ | | | | | | | |
| 2-6 | 91 (43) | 9.3 | 2.0-159.5 | | 501.06 | 99.4-1807.6 | |
| 7-10 | 122 (57) | 10.94 | 4.4-166.9 | .040 | 499.20 | 402.1-2068.3 | .843 |
| RP regional lymph | | | | | | | |
| node metastases | | | | | | | |
| Negative | 204 (95) | 9.6 | 4.0-2068.3 | | 476.90 | 402.1-2068.3 | |
| Positive | 11 (5) | 29.8 | 20.2-153.4 | <.001 | 611.50 | 490.2-1439.2 | .012 |

| | CC§ | P | CC§ | P |
|---|---|---|---|---|
| Age | 0.133 | .051 | 0.149 | .090 |
| Pre-operative PSA | 0.119 | .081 | −0.025 | .717 |
| Pre-operative VEGF | — | — | −0.005 | .940 |
| Pre-operative sVCAM-1 | −0.005 | .940 | — | — |
| RP tumor volume□ | 0.113 | .126 | 0.008 | .927 |

RP = Radical prostatectomy
CC = Correlation coefficient
‡RP extracapsular extension status, RP seminal vesicle involvement status, RP surgical margin status, and RP Gleason sum were not available for two patients, who did not undergo a prostatectomy because of positive pelvic lymph nodes at the time of surgery.
§Spearman's correlation coefficients.
□Radical prostatectomy tumor volume was unavailable for 61 prostatectomy patients

TABLE 19

| | Organ Confined Disease | | | Metastases to Regional Lymph Nodes | | |
|---|---|---|---|---|---|---|
| | Hazard Ratio | 95% CI | P | Hazard Ratio | 95% CI | P |
| Pre-operative VEGF | 0.997 | 0.988-1.006 | .528 | 1.036 | 1.018-1.053 | <.001 |
| Pre-operative sVCAM-1 | 1.000 | 0.999-1.001 | .455 | 1.002 | 0.999-1.004 | .090 |
| Pre-operative PSA* | 0.928 | 0.878-0.980 | .008 | 0.971 | 0.871-1.082 | .592 |
| Biopsy Gleason Sum† | 0.293 | 0.168-0.510 | <.001 | 2.603 | 0.553-12.247 | .226 |
| Clinical Stage | 0.771 | 0.580-1.025 | .073 | 2.584 | 1.167-5.720 | .019 |

*Pre-operative PSA level had a skewed distribution and therefore was modeled with a log transformation.
†Biopsy Gleason Sum was categorized as grade 2 to 6 versus grade 7 to 10.

Association of Pre-Operative Plasma VEGF and sVCAM-1 with Biochemical Progression after Radical Prostatectomy

Overall, 20% of patients (42 of 215) had cancer progression with a median post-operative follow-up of 60.1 months (range 2.5 to 86.3). The overall PSA progression-free survival was 86.0±2.4% (Standard error, SE) at 3 years, 79.3±3.0% (SE) at 5 years, and 76.9±3.3% (SE) at 7 years. On univariate and multivariate Cox proportional hazards regression analysis (Table 20), higher pre-operative plasma VEGF (P=0.005 and P=0.043, respectively) as well as biopsy Gleason sum >7 (P=0.001 and P=0.015, respectively) and pre-operative serum PSA (P<0.001 and P<0.001, respectively) were associated with the risk of PSA progression, when adjusted for the effects of clinical stage and pre-operative plasma sVCAM-1.

TABLE 20

| | Univariable | | | Multivariable | | |
|---|---|---|---|---|---|---|
| | Hazard Ratio | 95% CI | P | Hazard Ratio | 95% CI | P |
| Pre-operative VEGF | 1.009 | 1.003-1.016 | .005 | 1.008 | 1.000-1.015 | .043 |
| Pre-operative sVCAM-1 | 1.001 | 0.999-1.001 | .122 | 1.001 | 0.999-1.002 | .066 |
| Pre-operative PSA* | 1.067 | 1.043-1.092 | <.001 | 1.058 | 1.032-1.085 | <.001 |
| Biopsy Gleason Sum† | 2.891 | 1.572-5.315 | .001 | 2.223 | 1.168-4.229 | .015 |
| Clinical Stage | 0.915 | 0.684-1.224 | .548 | 0.879 | 0.651-1.188 | .402 |

*Pre-operative PSA level had a skewed distribution and therefore was modeled with a log transformation.
†Biopsy Gleason Sum was categorized as grade 2 to 6 versus grade 7 to 10.

Discussion

Patients with prostate cancer metastatic to bones had significantly elevated pre-operative plasma levels of VEGF and sVCAM-1 compared to patients with clinically localized disease or normal healthy controls. Pre-operative plasma levels of both VEGF and sVCAM-I were both significantly elevated in patients with lymph node involvement, however, only pre-operative VEGF was elevated in patients with biopsy and final Gleason score (SUM?)≧7 and extraprostatic extension. On univariate logistic regression analyses pre-operative plasma VEGF levels were associated with organ-confined disease and lymph node involvement, whereas pre-operative plasma sVCAM-1 were not. On multivariate logistic regression analyses (Table 18), pre-operative plasma VEGF was associated with prostate cancer involvement of the lymph nodes but not with confinement of the cancer to the prostate, when adjusted for the effects of standard pre-operative features and pre-operative plasma sVCAM-1. On univariate and multivariate Cox proportional hazards regression analysis (Table 17), higher pre-operative plasma VEGF as well as biopsy Gleason sum ≧7 and pre-operative serum PSA were associated with the risk of PSA progression, when adjusted for the effects of clinical stage and pre-operative plasma sVCAM-1.

Studies show increased local and circulating levels of VEGF in patients with advanced pathological stage prostate cancer (Jones et al., 2000; Kuniyasu et al., 2000; Chevalier et al., 2002). In accordance with Duque et al. (1999), markedly elevated VEGF in patients with prostate cancer metastasis was observed in the present study.

VEGF was significantly elevated in patients with lymph node involvement. VEGF was elevated in patients with biopsy Gleason grade ≧7, final Gleason grade ≧7, and extraprostatic extension. After radical prostatectomy, the majority of patients with organ-confined extracapsular disease and even seminal vesicle invasion, whose local tumor is completely removed as evidenced by a negative surgical margin, experience long-term freedom from biochemical progression (Epstein et al., 1998; Tefilli et al., 1998; Epstein et al., 2000).

VEGF was an independent predictor of biochemical progression after radical prostatectomy. In most patients with lymph node involvement local therapy for recurrence eventually fails, giving rise to sites of distant metastasis (Walsh et al., 1994; Catalona and Smith, 1998). Nomograms which can predict disease progression rather than simply pathologic features in patients who undergo radical prostatectomy for prostate cancer, that incorporate biomarkers, would be most useful in the management of patients with prostate cancer (Kattan et al., 1997). sVCAM has been shown to mark principally small blood vessels, probably tumor angiogenesis, in prostate cancer specimens (Wikstrom et al., 2002) and serum (Lynch et al., 1997). sVCAM-1 was found to be markedly elevated in patients with prostate cancer metastasis to bone. sVCAM-1 is an independent predictor of biochemical progression after radical prostatectomy, presumably due to an association with microscopic metastatic disease already present at the time of surgery.

Plasma VEGF and sVCAM-1 levels were highest in patients with bone metastases. In accordance with Kuniyasu et al. (2000), VEGF levels in prostatectomy specimens were found to be highest in pathologically advanced prostate cancers as well as those of high histological grade. In hormone refractory prostate cancer, George et al. (2001) suggested that elevated plasma levels of VEGF might not simply be a marker of the extent of disease but rather could define a specific biological phenotype, given that VEGF data were more significant in multivariate analysis controlling for markers of disease burden.

Within the group of prostatectomy patients, while pre-operative plasma VEGF and sVCAM-1 levels were elevated in patients with metastases to regional lymph nodes, only higher VEGF levels were associated with higher biopsy and final Gleason sum and extraprostatic extension. Higher pre-operative VEGF level was associated with lymph node involvement and biochemical progression, when adjusted for the effects of standard pre-operative features.

A possible confounding factor of the study, given the comorbidity of artherosclerosis in the patients in the study and its prevalence within the general male population, is that sVCAM-1 has been shown to be elevated in patients with artherosclerosis (De Caterina et al., 1997; Peter et al., 1999) as the serum level of sVCAM-1 appears to correlate with the extent of atherosclerosis. However, other authors refute this claim (Blann et al., 1998; de Lemos et al., 2000).

The present study was limited partly by the low rate of disease progression (20%) in the patient cohort after a median follow-up of 60.1 months, which yielded a 5-year progression free probability of 79.3%. The low progression rate in the studied population may be caused by the lower cancer stage and volume observed in more recent surgical series given wide based PSA-based screening. In other reported series, approximately 44% to 47% of men who underwent radical prostatectomy had pathologically nonorgan-confined disease (Partin et al., 1993; Wheeler et al., 1998), and in the present cohort, only 36.7 of cancers were not organ confined. The pathologic stage of prostate cancer is known to be a strong predictor of progression after radical prostatectomy (Epstein et al., 1996). Nevertheless, 34.7% of the studied patients had a pre-operative PSA level above 10 ng/mL, 34.4% had extraprostatic extension in their pathological prostatectomy specimen, and 57.3% had final pathologic Gleason sum of 7 or above, which is representative of patients who currently undergo radical prostatectomy for clinically localized prostate cancer. The positive margin rate in the present series was only 15.5%, which may have decreased the rate of progression attributable to local failure. Pre-operative PSA was associated with disease progression in the present study. The inclusion of many high range pre-operative PSA (>75^{th} percentile, 11.3 ng/mL) likely increased the predictive value of pre-operative PSA as reported in previous studies in radical prostatectomy patients (Catalona and Smith, 1998).

Viewed in the context of similar observations made for other cancers, these data support a relationship between elevated circulating VEGF and sVCAM-1 levels and metastatic cancer, particularly in bony metastasis. The biologic and prognostic implications of micrometastases need to be defined more accurately. Elevated VEGF and possibly sVCAM-1 seem to be associated with biologically active and clinically significant disease. Circulating levels of sVCAM-1 may be associated with a more complex relationship between development of metastatic potential and VEGF may be critical in the establishment of neo-vascularization at distant sites of metastasis, in addition to its classic role as a tumor marker. The predictive value of circulating VEGF levels remains significant even when controlled for other tumor-specific markers of biologically aggressive disease such as Gleason grade, tumor invasiveness, and PSA. VEGF and sVCAM-1 levels also seem to be associated with the presence of clinically undetected low-volume metastases. It remains unclear whether circulating VEGF or sVCAM-1 levels are produced by host factors such as distant organ response to invasion or are the result intrinsic tumor cell biologic activity. An improved understanding of the biologic mechanism for elevation of circulating VEGF and sVCAM-1 in patients with metastatic cancer would possibly allow improved clinical management of these patients and provide new targets for therapy and markers of to monitor anti-angiogenic therapies (Miller, 2002).

Plasma VEGF and sVCAM-1 levels are markedly elevated in men with prostate cancer metastatic to regional lymph nodes and bone. In men without clinical or pathologic evidence of metastases, the pre-operative plasma VEGF level is a strong predictor of biochemical progression after surgery, presumably because of an association with occult metastatic disease present at the time of radical prostatectomy.

Conclusions

Plasma VEGF and sVCAM-1 levels are markedly elevated in men with metastatic prostate cancer. Furthermore, both are independent predictors of biochemical progression after radical prostatectomy, presumably due to an association with microscopic metastatic disease already present at the time of surgery.

### EXAMPLE 7

Several studies have conclusively shown that standard sextant biopsy (S6C) detects fewer prostate cancers compared to biopsy templates that include additional, laterally-directed biopsy cores (Gore et al., 2001; Chang et al., 1998). For example, Gore et al. (2001) demonstrated that sextant biopsies detected only 69% of the cancers identified by a systematic 12-core biopsy (S12C) regimen that included 6 additional, laterally directed cores, one each at the base, mid-portion, and apex of the prostate in addition to standard S6C. Since S6C fails to detect approximately one-third of cancers present, it seems inevitable that S6C would also perform poorly in predicting pathologic features of the prostate following radical prostatectomy; in fact, many studies have confirmed the poor performance of S6C in predicting post-prostatectomy pathology. These studies have assessed the predictive value of various biopsy parameters, including biopsy GS, number of positive cores, percent of tumor in the biopsy specimen, and total length of cancer in S6C set in predicting pathologic features of the prostatectomy specimen. Sebo et al. (2000) reported that percent of cores positive for cancer and biopsy Gleason score of sextant biopsy were independent, significant predictors of tumor volume. However, in that study the correlation coefficients were 27% and 11.6% (R² multiplied by 100), respectively. In another study, although cancer volume significantly correlated with the number of positive biopsies, percent of positive biopsies, total cancer length in the biopsy specimen, and Gleason grade ⅘, all correlation coefficients were less than 10% (Noguchi et al., 2001).

Despite these significant associations between S6C biopsy parameters and prostatectomy pathology, reliable algorithms that include S6C biopsy parameters to predict extracapsular extension (ECE) (Egawa et al., 1998), tumor volume (Noguchi et al., 2001), and pathologic Gleason score (pGS) (Narain et al., 2001) have not emerged. Noguchi et al. (2001) reported that there was a weak and disappointing correlation among all pathological features of 6 systematic biopsies and radical prostatectomy specimens. Cupp et al. (1995) also demonstrated the poor performance of S6C biopsies in predicting pathologic parameters of the radical prostatectomy specimen.

Material and Methods

Patient Population

All 228 patients who underwent a S12C biopsy at a single institution (Scott Department of Urology, Baylor College of Medicine, Houston, Tex.) and a subsequent radical retropubic prostatectomy by a member of the full-faculty were potential candidates for this analysis. S12C biopsy became the standard initial biopsy technique for the Baylor Prostate Center faculty. Two men initially treated with definitive radiotherapy and forty-eight who had a history of a prostate biopsy prior to their S12C biopsy were excluded. This left one hundred seventy-eight (178) men for analysis.

Prostate Needle Biopsy Technique

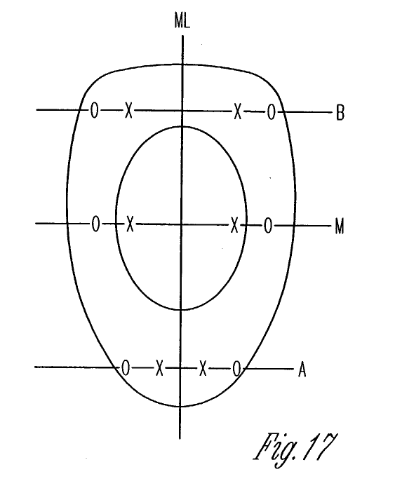

The S12C needle biopsy was performed as previously described (Gore et al., 2001). Briefly, a standard sextant biopsy as described by Hodge et al. (1989) was performed with the addition of laterally directed biopsies in the peripheral zone at the base, mid, and apex of the prostate (FIG. 17). Each biopsy core was individually identified as to its location of origin (base, mid, or apex; right or left; sextant or laterally-directed) using a 4-specimen cup technique and the use of red, green, and blue ink. Additional ultrasound, finger, or transitional zone directed biopsy cores performed at the discretion of the staff urologist were excluded from this study. All biopsies were performed in a standardized fashion by a staff urologist along with one of two ultrasound technicians, who served to help standardize the biopsy template across all patients. Gray scale transrectal ultrasonography was performed using the Hitachi (Hitachi Medical Systems, Tokyo, Japan) EUB-V33W 6.5 MHz end-fire probe. Biopsy cores were obtained using an 18 gauge needle with the ProMag (Manan Medical Systems, Northbrook, IL) 2.2 spring loaded gun. The entire prostate gland and transitional zone were measured in three dimensions, and the volume estimated using the prolate ellipsoid formula.

Pathology Specimens

In each biopsy specimen, the following variables were assessed and documented by a full-time faculty pathologist: total millimeter (mm) of cancer involvement of each core, total mm length of each core, and GS of the tumor identified in any core with tumor. Radical retropubic prostatectomies were performed at one of two teaching hospitals, either St. Luke's Episcopal Hospital (n=42), Houston, Tex., or The Methodist Hospital (n=136), Houston, Tex. Prostatectomy specimens at The Methodist Hospital were fixed and processed in the whole-mount technique with 5-mm transverse sections as previously described in Wheeler and Lebowitz (1994). Prostatectomy specimens at St. Luke's Hospital were serially sectioned into multiple levels and then subdivided into two or four pieces and submitted in entirety. pGS was assigned after review of the cross-sections. ECE was scored as a binary, categorical variable (with L3E and L3F considered positive, see Wheeler et al., 1998) after the extent of each cancer focus was identified. Total tumor volume (TTV) was calculated using a computerized planimetric method with Optimas software using the Bioscan image analysis system on all whole mount step sectioned prostatectomy specimens.

Prognostic Variables and Statistics

The comparison biopsy set groups included the sextant (FIG. 17, S6C=X), the laterally directed systematic six cores (FIG. 17, L6C=O), and entire S12C biopsy set (FIG. 17, S12C=X+O). The percent of tumor involvement per biopsy set was derived using the formula: ((total percent of tumor in core 1)+(total percent of tumor in core 2)+(total percent of tumor in core 3)+ . . . /(total number of cores in the set))×100. The total cancer length of a biopsy set was the sum of all mm of cancer in that particular biopsy set. Biopsy GS was determined as the sum of the maximum primary and secondary Gleason grades for the biopsy set. Biopsy GS, number of positive cores, total length of cancer, and percent of tumor in each biopsy set group were examined for their ability to predict ECE, TTV, and pGS with Spearman's rho correlation coefficients.

Stepwise multiple regression analyses were performed to determine independent predictors of the prostatectomy pathology. Biopsy parameters from both the L6C and S6C sets were included this analysis. S12C set biopsy predictors were not included in this analysis because these parameters are not independent of the S6C and 6LC parameters, but simply mathematical manipulations of them. For instance, the S12C number of positive cores and total cancer length are the addition of the L6C and S6C parameters, the percent of tumor involvement is the addition of L6C and S6C percent tumor involvement divided by two, and the S12C biopsy GS is the sum of the maximum primary and secondary grades contained in the L6C and S6C sets. Statistical significance in this study was set as P<0.05. All reported P values are two-sided. All analyses were performed with the SPSS statistical package (SPSS version 10.0 for Windows).

The independent biopsy predictors of ECE, pGS, and TTV were utilized to construct a test to evaluate the sensitivity, specificity, and positive and negative predictive values for the presence of insignificant cancer as defined by described by Epstein et al. (1998). Specifically, insignificant tumors were defined as having a tumor volume of <0.5 cm³, confined to the prostate, and having a pGS less than 7. To minimize bias, the median results of the biopsy predictor variables were used as the cut-point values.

Results

The median age for the study cohort was 62 years, and the median total and % free PSA were 5.8 ng/ml and 24.7, respectively. The median TTV was 0.56 cc. 24.7% of the patients had ECE (Table 21). S12C set-derived parameters demonstrated the highest correlation coefficients in predicting ECE and TTV (Table 22). The sextant set Gleason score best predicted pGS followed by the S12C set Gleason score. The greatest coefficient for predicting TTV for each of the biopsy sets was total cancer length (S12C>L6C>S6C). Percent tumor involvement, total cancer length, and number of positive cores in the S12C were better predictors of ECE than any biopsy parameter derived from the L6C or S6C sets. Collectively, the correlation analyses showed a superior association between S12C-derived parameters and both TTV and ECE when compared to S6C or L6C-derived parameters.

TABLE 21

| Characteristic | n = 178 |
|---|---|
| Median age (yrs.; interquartile range) | 62 (57-67) |
| Median PSA (ng./ml; interquartile range) | 5.8 (4.1-8.0) |
| Median free PSA (%; interquartile range) | 12.1 (7.9-16.3) |
| Abnormal DRE (%) | 24.7 |
| Median transitional zone volume (cc.; interquartile | 18.0 (12.0-31.0) |
| range) | |
| Median prostate volume (cc.; interquartile range) | 40.0 (30.0-57.0) |
| Median total tumor volume (cc.; interquartile range) | 0.56 (0.19-1.09) |
| Extracapsular extension (%) | 24.7 |
| Pathologic Gleason score (%) | |
| ≦6 | 47.8 |
| 7 | 46.6 |
| ≧8 | 5.6 |

TABLE 22

| | | Extracapsular extension | | | | | |
|---|---|---|---|---|---|---|---|
| | Biopsy set | (n = 178) | | Pathologic Gleason* (n = 178) | | Total tumor vol. (n = 136) | |
| | predictors | Coefficient | P Value | Coefficient | p Value | Coefficient | p Value |
| 12 core set | Gleason score | 0.334 | <0.001 | 0.493 | <0.001 | .323 | <0.001 |
| | No. positive cores | 0.447 | <0.001 | 0.271 | <0.001 | .536 | <0.001 |
| | Total Ca. length | 0.474 | <0.001 | 0.296 | <0.001 | .615 | <0.001 |
| | % tumor | 0.482 | <0.001 | 0.328 | <0.001 | .597 | <0.001 |
| | involvement | | | | | | |
| Sextant set | Gleason score | 0.428 | <0.001 | 0.596 | <0.001 | 0.350 | <0.001 |
| | No. positive cores | 0.333 | <0.001 | 0.178 | 0.018 | 0.416 | <0.001 |
| | Total Ca. length | 0.406 | <0.001 | 0.256 | 0.001 | 0.475 | <0.001 |
| | % tumor | 0.405 | <0.001 | 0.283 | <0.001 | 0.472 | <0.001 |
| | involvement | | | | | | |
| Lateral 6 | Gleason score | 0.276 | <0.001 | 0.405 | <0.001 | 0.229 | 0.019 |
| core set | No. positive cores | 0.343 | <0.001 | 0.246 | 0.001 | 0.498 | <0.001 |
| | Total Ca. length | 0.324 | <0.001 | 0.227 | 0.002 | 0.566 | <0.001 |
| | % tumor | 0.320 | <0.001 | 0.249 | 0.001 | 0.545 | <0.001 |
| | involvement | | | | | | |

*Pathologic Gleason score was categorized as <7 versus ≧7.

In multivariable analyses that controlled for biopsy parameters of the sextant and the L6C set, contributions from both the S6C and the L6C set were associated with TTV, ECE, and pGS7 or greater (Table 23). The S6C Gleason score and number of positive lateral cores each had a greater than two-folds odds of predicting ECE. S6C Gleason score had twelve-fold odds ratio of predicting pGS, far greater than L6C (two-fold) or S6C (less than one-half-fold) number of positive cores. The S6C % tumor involvement and L6C total cancer length each independently predicted TTV.

Thirty-three (20.1%) of the patients in this study met Epstein's criteria (Epstein et al., 1994) for insignificant tumor. Using a test derived from the S6C parameters, 45 patients were incorrectly categorized as having insignificant cancer (Table 24). However, by adding the L6C parameters, only 10 patients were incorrectly categorized as having pathologic features indicative of insignificant cancer. Thus, the combination of S6C and L6C parameters increased the positive predictive value from 39% to 52% with only an 11% drop in the % negative predictive value. Alternatively, the S6C biopsy based test incorrectly categorized the significance of 49 (29.9%) tumors, as compared to the S12C based test which incorrectly categorized only 32 (19.5%) of tumors.

TABLE 23

| | Extracapsular extension | | | | | | | | |
|---|---|---|---|---|---|---|---|---|---|
| | (n = 178) | | | Pathologic Gleason score (n = 178)* | | | Total tumor volume (n = 136) | | |
| | Hazard | | p | Hazard | | p | Parameter | | |
| | Ratio | 95% CI | Value | Ratio | 95% CI | Value | Estimate | 95% CI | p Value |
| Sextant set | | | | | | | | | |
| Gleason score | 2.624 | 1.480-4.654 | 0.001 | 12.200 | 4.003-37.180 | <0.001 | | | 0.702 |
| No. Positive | | | 0.444 | 0.415 | 0.211-0.814 | 0.010 | | | 0.474 |
| cores | | | | | | | | | |
| Total cancer | | | 0.418 | | | 0.870 | | | 0.963 |
| length | | | | | | | | | |
| % Tumor | | | 0.090 | | | 0.057 | 0.066 | 0.037-0.095 | <0.001 |
| involvement | | | | | | | | | |
| Lateral 6 core set | | | | | | | | | |
| Gleason score | | | 0.978 | | | 0.169 | | | 0.749 |
| No. Positive | 2.283 | 1.375-3.791 | 0.001 | 2.071 | 1.082-3.962 | 0.028 | | | 0.627 |
| cores | | | | | | | | | |
| Total cancer | | | 0.178 | | | 0.582 | 0.005 | 0.001-0.009 | 0.022 |
| length | | | | | | | | | |
| % Tumor | | | 0.188 | | | 0.930 | | | 0.190 |
| involvement | | | | | | | | | |

*Pathologic Gleason score was categorized as <7 versus ≧7.

TABLE 24

| | | No. | No. non- | % Positive | % Negative | | |
|---|---|---|---|---|---|---|---|
| | | insignificant | insignificant | predictive | predictive | | |
| | | tumors (%) | tumors (%) | value | value | % Sensitivity | % Specificity |
| Sextant biopsy | | | | | | | |
| parameters | | | | | | | |
| Favorable | Sextant Gleason score <7 | 29 (17.7) | 45 (27.4) | 39 | | | |
| | and sextant Ca. | | | | | | |
| | involvement ≦4% | | | | | | |
| Unfavorable | Sextant Gleason score ≧7 | 4 (2.4) | 86 (52.4) | | 96 | 88 | 66 |
| | or sextant Ca. involvement | | | | | | |
| | >4% | | | | | | |
| Sextant and | | | | | | | |
| laterally directed | | | | | | | |
| biopsy parameters | | | | | | | |
| Favorable | Sextant Gleason score <7 | 11 (6.7) | 10 (6.1) | 52 | | | |
| | and sextant Ca. | | | | | | |
| | involvement ≦4% and ≦1 | | | | | | |
| | lateral positive core and | | | | | | |
| | total lateral Ca. length ≦3 mm | | | | | | |
| Unfavorable | Sextant Gleason score ≧7 | 22 (13.4) | 121 (73.8) | | 85 | 33 | 92 |
| | or sextant Ca. involvement | | | | | | |
| | >4% or >1 lateral positive | | | | | | |
| | core or total lateral Ca. | | | | | | |
| | length >3 mm | | | | | | |

Discussion

Variables closely associated with the outcome of interest underlie the development of nomograms with greater discriminatory ability and calibration. Building on previous work in this area (Sebo et al., 2000; Noguchi et al., 2001; Epstein et al., 1994; Grossklaus et al., 2002), it was determined whether the data in an extended field biopsy could enhance post-prostatectomy pathology prediction. It was hypothesized that the addition of the laterally directed biopsies to standard systematic sextant biopsy provides unique post-prostatectomy pathology predictive value. The analyses described herein demonstrated that the laterally directed biopsy cores contained unique information, improving the prediction of ECE, pGS, and TTV in prostatectomy specimens, in multivariable analyses that included biopsy information from the sextant set. This represents an advancement in the understanding of biopsy predictors of prostate pathology, and provides the rationale for incorporating extended field biopsy data in future prediction models and nomograms.

The study population represents a current cohort of patients with clinically localized prostate cancer detected with a S12C biopsy. While the superiority of S12C over sextant biopsy has been gaining acceptance, few studies have addressed the respective performance of various biopsy templates in predicting final pathologic parameters after radical prostatectomy. Taylor et al. (2002) reported recently that a greater number of significant cancers (defined as not <0.2 cc, organ confined, and pGS<7) are detected with an extended field biopsy. Sebo et al. (2000) recently reported that in prostate cancer patients diagnosed between March 1995 and April 1996 with an average of 6.2 cores, 20.8% had a tumor volume of less than 0.5 cc. In the present cohort, nearly one-half of the patients had a tumor volume of less than 0.5 cc, although some of these had a final GS of >7. The increase in the proportion of smaller tumors detected is likely due to the fact that the study population was biopsied with a systematic 12-core biopsy. Multiple authors have demonstrated continuing stage migration to smaller, less advanced tumors in more recently diagnosed patients cohorts. In addition, there may be an increased likelihood of detecting small tumors with the addition of laterally directed cores. The rate of ECE in our cohort was, however, only marginally less than that reported by Sebo et al. (2001) (24.7% versus 26.6%). The median age and PSA of the cohort compares similarly to recent reports in which patients have undergone a mean of 10 or more core biopsies (San Franasco et al., 2003; Presti et al., 2003). In aggregate, these data suggest that, on average, smaller tumors diagnosed with a S12C exhibit a similar proportion of features of aggressive cancer, as those diagnosed with sextant biopsy.

TTV, pGS, and ECE were chosen as outcome variables because they represent the best pathologic predictors for prostate cancer recurrence and indolence in patients without seminal vesicle invasion or lymph node involvement (Wheeler et al., 1998; Koch et al., 2000; Epstein et al., 1993). Over the last several years, various groups have suggested that the percent of cancer in the biopsy represents the best predictor of pathology findings after prostatectomy (Grossklaus et al., 2002; Sebo et al., 2001), whereas others have proposed that the number of positive cores (Wills et al., 1998) or the total mm of cancer in the biopsy specimen (Goto et al., 1998) best indicates prostate pathology. Mindful of these contradictory findings, it was elected to evaluate a broad range of biopsy predictors: number of positive cores, % of cancer involvement, total cancer length, and biopsy Gleason score. In designing this study, it was attempted to minimize the bias favoring the predictive potential of the L6C set. Therefore, patients with a history of biopsy prior to their S12C set were excluded, because many of these patients would have had a prior negative sextant biopsy.

In univariate correlation analyses, all the biopsy parameters from the S12C, S6C, and L6C set were significantly associated with TTV, ECE, and pathologic GS. Consistent with the hypothesis, the highest coefficients for predicting TTV and ECE were derived from the S12C set, suggesting that information contained in the S12C set is more representative of what is found in the prostatectomy specimen. Despite the superiority of the S12C, a significant correlation of the S6C with final pathologic parameters was found, consistent with previous studies based primarily on patients who had sextant biopsy. For example, Noguchi et al. (2001) demonstrated in a univariate analysis that the number of positive biopsy cores and total cancer length were significantly associated with cancer volume and the positive surgical margin rate. Sebo et al. (2000), analyzing 210 patients who underwent radical prostatectomy, found that the percent of tumor involvement and biopsy GS were significant predictors of pathologic stage.

It was further determined which of the biopsy-based parameters were independent predictors of prostate pathology in multivariable analyses. It was found that S6C and the L6C set both contributed significantly to the prediction of ECE, pGS (<7 vs. >7), and TTV. The significant S6C set biopsy parameters, which emerged in the multivariable analyses, were consistent with previous reports based on non-extended field biopsy schemes. Gilliland et al. (1999) reported that biopsy Gleason score independently predicted ECE status, a finding in congruence with the present S6C set Gleason score. pGS was best predicted by the S6C Gleason score with a greater than 12-fold odds. Interesting, an odds ratio of less than one-half was associated with the number of positive S6C cores in predicting pGS. This implies that if all else is kept equal, a greater number of positive sextant cores predicts a lower pathologic Gleason score. This finding could be explained by a greater sampling of the transition zone in the S6C than in the L6C set. Transitional zone tumors are less biologically aggressive and are generally associated with a lower Gleason score at the time of diagnosis (Mai et al., 2001) than peripheral zone tumors.

The L6C number of positive cores, notably, added a greater than two-fold odds in predicting ECE and pGS. The % tumor involvement of the S6C set predicted TTV, in agreement with the findings of Grossklaus et al. (2002) and Sebo et al. (2000). The L6C total cancer length contributed to the prediction of TTV independently of the S6C % tumor involvement. As compared to the original systematic sextant approach described by Hodge, the biopsy technique with laterally directed biopsies sampled more of the peripheral zone, an area more likely to harbor cancer. In particular, the S12C set included the highest cancer detection sites, such as the lateral apex and lateral base (Gore et al., 2001), likely resulting in a better assessment of the prostate tumor present.

Although there is clear evidence that a nomogram outperforms a stratifying risk model (Eastham et al., 2002), to gain preliminary insight into the value contained in the S12C set, a test was constructed for tumor insignificance based on Epstein's criteria (Epstein et al., 1994). It appears that addition of the laterally directed biopsy data to such a test improves its specificity and positive predictive value and decreases the incorrect categorization of tumor significance by 10.4%. This finding suggests that utilizing S12C based parameters would allow the physician to identify patients with insignificant tumor burden while minimizing the risk of under treating patients with significant tumors. One could potentially improve the robustness of a nomogram based on an extended field biopsy set with the addition of clinical and biomarker data.

Conclusion

The present study provides evidence that the total number of biopsy cores, and the location from which each core is obtained, greatly influences the accuracy of biopsy predictors of post-prostatectomy pathology. In the present cohort, both the S6C and L6C set independently contributed to the prediction of pathologic Gleason score, total tumor volume, and extracapsular extension. Pre-operative nomograms that utilize S12C data and specify biopsy parameters obtained from sextant and laterally directed biopsy cores will likely demonstrate improved performance in predicting post-prostatectomy pathology (e.g., indolent cancer or the presence of extracapsular extension).

### EXAMPLE 8

Validated cut-points for percent free PSA (% fPSA) and PSA density (PSAD) are based on cancer detection using primarily sextant biopsies. Systematic 12-core (S12C) biopsies that include standard sextant plus six laterally-directed biopsies significantly increase the detection rate for prostate cancer, and may detect a greater proportion of small volume cancers. PSA elevations that prompt biopsy in these patients, may be due to benign prostatic hyperplasia (BPH) rather than cancer.

Methods

This study evaluated 336 consecutive men whose PSA ranged between 4 and 10 (ng/ml) and who underwent a S12C biopsy. The medial 6-core biopsies (M6C) and the full S12C set comprise the study groups. Finger and ultrasound directed biopsy cores were excluded. ROC curves for PSATZD (PSA transition zone density), PSAD (PSA density), total PSA (tPSA), complexed PSA (cPSA), and % fPSA were constructed based on cancer diagnosis, and the AUCs were compared. In addition, the 90% sensitivities with their respective cut-points and specificities were calculated.

Results

The cancer detection rate was 37.7% and 28.4% for the S12C and M6C biopsy sets, respectively. Of note, for both biopsy study groups, PSATZD performed better than PSAD, which in turn performed better than % fPSA. The AUCs and 90% sensitivity values for the S12C and M6C groups are shown below.

TABLE 25

S12C

90% sensitivity

| | AUC | cutpoint | specificity |
|---|---|---|---|
| PSATZD | 0.688 | 0.1000 | 0.131 |
| PSAD | 0.671 | 0.0634 | 0.165 |
| % fPSA | 0.600 | 23.05 | 0.16 |
| cPSA | 0.539 | 3.5996 | 0.117 |
| tPSA | 0.513 | 4.450 | 0.131 |

M6C

90% sensitivity

| | AUC | cutpoint | specificity |
|---|---|---|---|
| PSATZD | 0.719 | 0.1357 | 0.326 |
| PSAD | 0.696 | 0.0664 | 0.205 |
| % fPSA | 0.636 | 22.15 | 0.188 |
| cPSA | 0.548 | 3.5996 | 0.113 |
| tPSA | 0.511 | 4.450 | 0.13 |

The performance of the three serum tests with the greatest AUC, PSATZD, PSAD, and % fPSA, appears to be degraded with a S12C biopsy compared to the traditional sextant biopsy.

### EXAMPLE 9

To examine the predictors of prostate cancer on a second systematic 12-core biopsy (S12C) in patients with an initial S12C without evidence of prostate cancer, the study evaluated 1,047 consecutive patients who underwent an initial S12C biopsy. 144 of these patients had a S12C without evidence of prostate cancer and underwent a repeat S12C biopsy. Of these patients, 95 had a prostate serum antigen (PSA) at initial biopsy between 2.5 and 10 ng/ml and ultimately comprised the study population. Parameters that were evaluated included initial and repeat biopsy PSA, initial and repeat percent free PSA (% FPSA), initial and repeat biopsy digital rectal exam (DRE) status (normal versus abnormal), presence of high grade prostatic intraepithelial neoplasia (PIN) on initial biopsy, presence of atypical small acinar proliferation (ASAP) on initial biopsy, poor DRE change (initial normal→repeat abnormal), PSA doubling-time (PSAdt=log(2)*(number of days between PSA measurement)/[log(repeat PSA)−log(initial PSA)]), and yearly inter-biopsy PSA changes (yibPSA=[(repeat PSA)−(initial PSA)]/(number of days between PSA measurement)*365). Statistical methods included the Mann-Whitney U test, Pearson Chi-Square test, and multivariable logistic regression analysis.

Results

In univariable analyses PSAdt, yibPSA, initial and repeat PSA, initial and repeat % FPSA, poor DRE change, repeat DRE status, and presence of ASAP were not significant predictors of prostate cancer at repeat biopsy. However, both initial DRE status (P=0.034) and the presence of PIN (P=0.010) were significant predictors of prostate cancer at repeat biopsy. In multivariable logistic regression analysis, only the presence of PIN remained a significant predictor of prostate cancer (P=0.012).

Conclusions

The results suggest that for patients with a PSA between 2.5 and 10 ng/ml whose initial S12C biopsy contains PIN but not cancer, the presence of PIN alone is an indication to re-biopsy.

### EXAMPLE 10

To determine whether data obtained through biopsy can be used to help predict side-specific posterolateral ECE, and whether a systematic, 12-core biopsy regimen (S12C) outperforms a S6C, 181 consecutive patients who underwent a S12C followed by radical retropbital prostatectomy (RRP) were analyzed. RRP specimens were processed using the whole-mount method. PSA, DRE, maximum biopsy Gleason Grade (mGG), number of positive cores (PC), number of contiguous positive cores (CPC) and percent of the biopsy material with cancer (%CA) were tested for their ability to predict posterolateral ECE using multivariate logistic regression analysis, and the Pearson Chi-Square test.

Results

The majority of the patients in the dataset with posterolateral ECE, had this as the only adverse pathologic feature of their prostate cancer. Only 19% (95% CI=1-33%) also had positive lymph nodes SVI, or ECE at the bladder neck or apex. Only 8% (CI=2-25%) had additional adverse pathological features when limited to those with a PSA<10 ng/ml and biopsy GS<7. Although in multivariate analyses controlling for DRE and mGG, the number of PC, %CA, and the number of CPC in the sextant cores were all predictors of ECE, on addition of the corresponding parameters from S12C data, these predictors were no longer significant, indicating that for each of the three parameters, S12C data was superior to sextant core data. The AUC of 12CR % CA was 0.88 (95% CI=0.82-93). S12C CPC and number of PC had sensitivities and specificities comparable to %CA.

Thus, data obtained through a S12C biopsy were independent predictors of posterolateral ECE and were superior to analogous sextant biopsy data.

### EXAMPLE 11

To develop a nomogram to predict the side of ECE in RP, 763 patients with clinical stage T1c-T3 prostate cancer who were diagnosed with a systematic biopsy and were subsequently treated with RP were studied. A ROC analyses were performed to assess the predictive values of each variable alone and in combination. The variables included an abnormality on DRE, the worst Gleason score (worst Gleason score in any one core), number of cores with cancer, percent cancer in a biopsy specimen (PERCA) on each side and serum PSA level.

Results

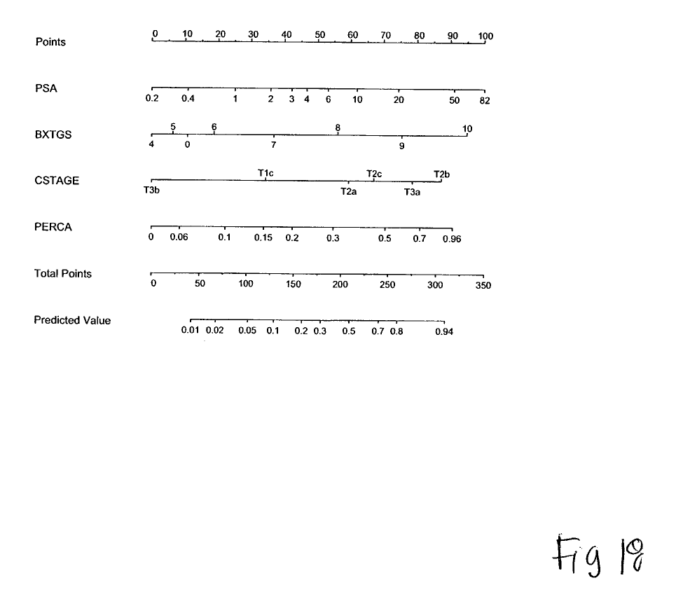

Overall, 31% of the patients had ECE and 17% of the 1526 sides of the prostate had ECE. Of the 812 sides with no palpable abnormality on DRE, 95 (11.5%) had ECE at the ipsilateral side compared to 20 (58.8%) of 34 sides with T3 nodule. Of the 500 sides with no cancer in a biopsy (recorded as Gleason sum 0), 30 (6%) had ECE at the ipsilateral side compared to 64 (52.4%) of 122 sides with Gleason sum 7 (4+3) 10 cancers. The area under the curve (AVC) of DRE, biopsy Gleason sum and PSA in predicting the side of ECE was 0.648, 0.724 and 0.627, respectively, and was 0.763 when these parameters were combined. Further, this was enhanced by adding the information of systematic biopsy with the highest value of 0.787 with a percent cancer. Based on the regression analysis, the nomogram was constructed (FIG. 18) and the accuracy of this nomogram was confirmed by the internal calibration.

Conclusions

A nomogram incorporating pre-treatment variables on each side of the prostate can provide accurate prediction of the side of ECE in RP specimens. Thus, this nomogram can assist the clinical decision such as resection or preservation of neurovascular bundle prior to radical prostatectomy.

### EXAMPLE 12

To develop a nomogram to improve the accuracy of predicting the freedom from PSA progression after salvage external beam radiotherapy (XRT) for biochemical recurrence (BCR) following radical prostatectomy (RP), pre- and post-prostatectomy clinical-pathological data and disease follow-up for 375 patients receiving salvage XRT was modeled using Cox proportional hazards regression analysis. Indications for salvage XRT included persistently elevated PSA following prostatectomy (n=108) and BCR(PSA>0.1, N=267) with or without clinically evident LR (local recurrence). Biochemical progression after salvage XRT was defined as two consecutive PSA rises greater than 0.1. Pre-radiotherapy variables were selected for use in the nomogram. These included pre-operative PSA, pre-XRT PSA, pre-XRT PSA doubling time, Gleason sum, pathological stage, surgical margins status, time from RP-to-BCR, neoadjuvant hormonal therapy and XRT dose.

Results

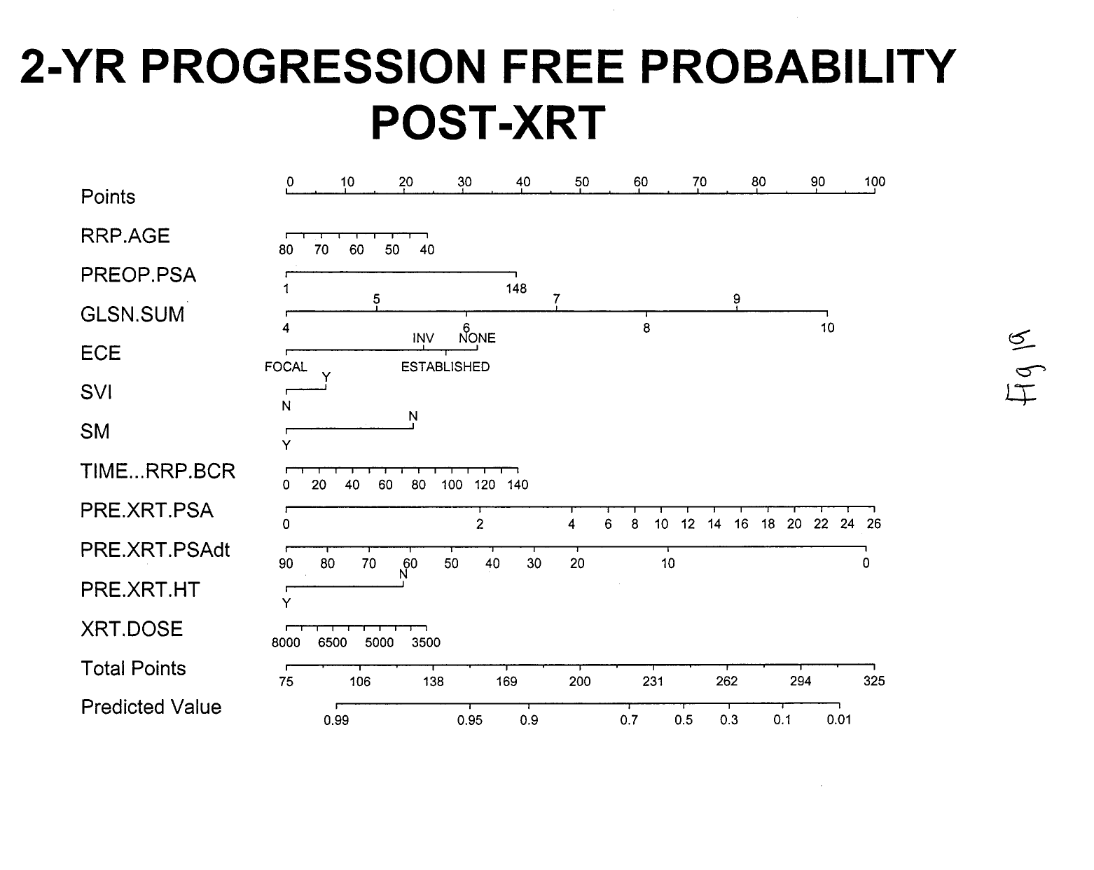

The median follow-up after XRT was 35.8 months. Overall, the 2-year and 5-year actuarial progression-free probability (PFP) after salvage XRT was 57% and 31% respectively. The median freedom from progression was 32.2 months. The median time-to-recurrence after XRT was 11.6 months. Multivariate Cox regression analysis revealed Gleason sum (HR 13.9, P=0.0002), pre-XRT PSA (HR 2.2, P=0.001), PSA doubling time (HR 0.45, P=0.002), positive surgical margins (HR 0.54, P=0.003) and neoadjuvant hormone therapy (HR 0.54, P=0.003) as significant prognostic variables. A nomogram to predict the 2-year progression-free probability was generated using all pre-selected variables (FIG. 19). The nomogram had a bootstrap-corrected concordance index of 0.73.

Given the morbidity and that a minority of patients derived a durable benefit from salvage radiotherapy in this cohort, it is evidence that patient selection is critical when considering this therapy. This nomogram is a tool to aid in identifying the most appropriate patients to receive salvage radiotherapy. The nomogram predicts a 2-year PFP between 65-95% for a typical patient with a pre-XRT PSA<2 ng/mL, PSADT>10 months, Gleason sum 2-7 and pT3a prostate cancer following salvage radiotherapy.

### EXAMPLE 13

To determine whether the transition zone volume (TZV) and total prostate volume (TPV) are independent predictors of PSA, 560 men who underwent a systematic 12-core biopsy performed under ultrasound guidance were analyzed, among a multi-racial population with and without positive prostate biopsies from total population (n=1047) of men who in a retrospective cohort study. Entry criteria were collection and analysis of pre-biopsy serum for determination of total and free serum PSA. TZV and TPV were calculated using the standard elliptical formula=height×width×length×0.524. Multivariable logistic and multivariate linear regression analyses were used to determine if race, age, TZV, and TPV were independent predictors and risk factors of total PSA, free PSA and highest quartile of total PSA.

Results

Of the 560 men in the cohort, 80%, were Caucasian, 4% were African-American, 5.2% Hispanic 9% Asian, and 14.8% were of mixed or “other” designations.

TABLE 26

| Variables in | | | | | | | |
|---|---|---|---|---|---|---|---|
| Logistic Regression | | Odds | | Variables in Logistic | | | Confidence |
| Analysis | p value | Ratio | Confidence Interval | Regression Analysis | p value | Odds Ratio | Interval |
| Race | 0.2667 | 1.097 | 0.93-1.29 | Race | 0.2667 | 1.084 | 0.92-1.28 |
| Age | 0.0036 | 1.054 | 1.02-1.09 | Age | 0.0036 | 1.048 | 1.01-1.09 |
| Biopsy Status | 0.0200 | 1.981 | 1.11-3.52 | Biopsy Status | 0.0200 | 2.143 | 1.19-3.85 |
| High TZV | 0.0003 | 3.06 | 1.74-5.64 | High TPV | <0.0001 | 4.148 | 2.26-7.63 |

When controlling for race, age and biopsy status using linear regression analysis, TZV and TPV are each separately significant predictors of PSA (P<0.0001 each) among men with either positive or negative systematic 12-core biopsies. Race did not prove to be an independent predictor of PSA in this study population.

### EXAMPLE 14

Men diagnosed with clinically localized prostate cancer have a number of treatment options available, including watchful waiting, radical prostatectomy and radiation therapy. With the widespread use of serum PSA testing, prostate cancers are being diagnosed at an earlier point in their natural history, with many tumors being small and of little health risk to the patient, at least in the short-term. To better counsel men diagnosed with prostate cancer, a statistical model that accurately predicts the presence of cancer based on clinical variables (serum PSA, clinical stage, prostate biopsy Gleason grade, and ultrasound volume), and variables derived from the analysis of systematic biopsies, was developed.

Materials and Methods

The analysis included 1,022 patients diagnosed through systematic needle biopsy with clinical stages T1c to T3 NO or NX, and MO or MX prostate cancer who were treated solely with radical prostatectomy at one of two institutions. Additional biopsy features included number and percentage of biopsy cores involved with cancer and highgrade cancer, in addition to total length of biopsy cores involved. Indolent cancer was defined as pathologically organ confined cancer, ≦0.5 cc in volume, and without poorly differentiated elements. Logistic regression was used to construct several prediction models and the resulting nomograms.

Results

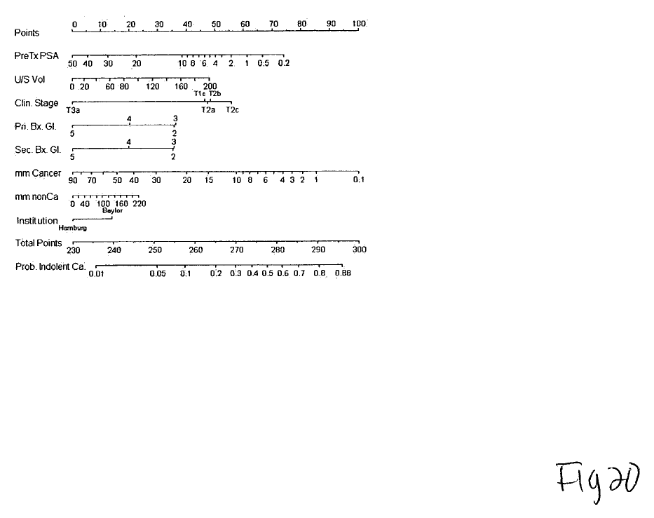

Overall, 105 (10%) of the patients had indolent cancer. The nomogram (FIG. 20) predicted the presence of an indolent cancer with discrimination (area under the receiver operating characteristic curves) for various models ranging from 0.82 to 0.90. Calibration of the models appeared good.

Conclusions

Nomograms incorporating pre-treatment variables (clinical stage, Gleason grade, PSA, and the amount of cancer in a systematic biopsy specimen) can predict the probability that a man with prostate cancer has an indolent tumor. These nomograms have excellent discriminatory ability and good calibration and may benefit both patient and clinician when the various treatment options for prostate cancer are being considered.

### EXAMPLE 15

To assess the prognostic significance of the sites of +SM in RP specimens, 1368 consecutive patients who were treated with RP by 2 surgeons were studied. Detailed pathologic features of cancer were assessed by one pathologist. The adjuvant radiation therapy before PSA recurrence was assessed as a time-dependent covariate to analyze PSA progression free probability (PFP). Median follow-up was 48 months.

Results

Overall, 179 patients (13%) had +SM. Of the 169 patients with the detailed results of +SM sites, 122 (73%) had only single +SM site, 32 (19%) had 2 sites and 14(8%) had >2 +SM sites. PFP at 5 year for patients with a single or 2 +SM sites was 71% and 74%, significantly better than 36% of patients with >2 +SM sites (p=0.006 and p=0.02, respectively). Of a total of 246 +SM sites, 30% were in the apical shave sections 29% in the apex (first two whole mount step sections), 24% in the mid, 9% in base section (last two sections), 6% in bladder neck, and 2% over seminal vesicles. In the analysis of the transverse section, 24% were in the anterior, 19% in the postero-lateral 14% in the posterior, 5% in the lateral. PFPs at 5 years for patients with a single +SM in the apical was 69% and in the apex, 84%, significantly better than 27% with a single +SM at the base (p=0.008 and p=0.01, respectively) while the patients with +SM in mid or bladder neck had an intermediate PFPs. More cancers were confined to the prostate when the +SM was at the apical (83%) or apex (74%) than at the base (14%). PFPs at 5 years for patients with a single +SM in the posterior was 48%, significantly worse than 79% of the patients with +SM in the anterior (p=0.033). In a Cox hazard regression analysis for the various models, +SM in the apical was only significant predictor of PSA progression (p=0.0021) when other established pathological features and serum PSA level were controlled. The +SM rate significantly decreased over the time as did the number of sites of +SM per prostate (p<0.005). Also the proportion of all +SM that were apical or apex significantly increased (p<0.005).

Conclusions

Prognostic significance of +SM may depend on the location of +SM in RP specimens. Although patients with +SM in the base and/or in the posterior had a worse PFP than other +SM locations, +SM in the apical shave sections, which has been significantly increasing, was the only significant predictor in a multivariate analysis. Thus, more attention should be paid for +SM in apical sections.

### EXAMPLE 16

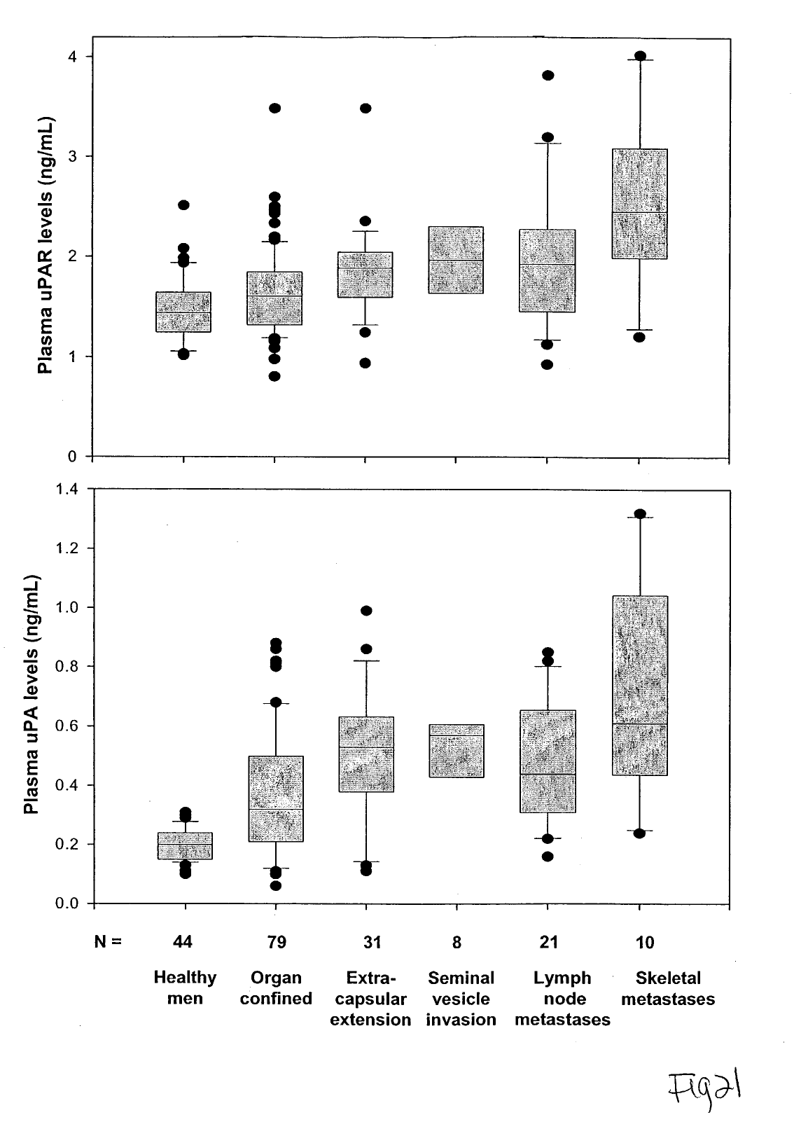

The urokinase plasminogen activation cascade has been closely associated with poor clinical outcomes in a variety of cancers. The following hypothesis was tested: that pre-operative plasma levels of the major components of the urokinase plasminogen activation cascade (urokinase plasminogen activator, UPA; the UPA receptor, UPAR; and the inhibitor, PAI-1) would predict cancer presence, stage, and disease progression in patients undergoing radical prostatectomy (FIG. 21).

Plasma levels of UPA, UPAR, and PAI-1 were measured pre-operatively in 120 consecutive patients who underwent radical prostatectomy for clinically localized disease and post-operatively in 51 of these patients. Marker levels were measured in 44 healthy men, in 19 patients with metastases to regional lymph nodes, and in 10 patients with bone metastases.

UPA and UPAR levels but not PAI-1 levels were elevated in prostate cancer patients compared with healthy subjects (P=0.006 and P=0.021, respectively) and were highest in patients with bone metastases. Elevated UPA and UPAR levels were associated with extraprostatic disease (P=0.046 and P=0.050, respectively) and seminal vesical involvement (P=0.041 and P=0.048, respectively). Elevated UPA and UPAR levels were correlated with prostatic tumor volume (P=0.036 and P=0.030, respectively). In multivariate analysis, pre-operative plasma UPA and UPAR levels, as well as biopsy Gleason sum, were independent predictors of prostate cancer progression (P=0.034, P=0.040, and P=0.048, respectively). In patients with disease progression, pre-operative plasma UPA and UPAR levels were higher in those with features of aggressive than in those with features of non-aggressive failure (P=0.050 and P=0.031, respectively).

While plasma UPA and UPAR levels were elevated in men with prostrate cancer compared to healthy men, they were most dramatically elevated in men with bony metastases. Pre-operative plasma levels of UPA and UPAR levels were associated with established features of biologically aggressive prostate cancer and disease progression. In multivariate analysis, pre-operative UPA and UPAR levels were independent predictors of disease progression in men undergoing radical prostatectomy. In combination with other clinical and pathologic parameters, plasma UPA and UPAR levels may be useful in selecting patients to enroll in clinical neo-adjuvant and adjuvant therapy trials.

### EXAMPLE 17

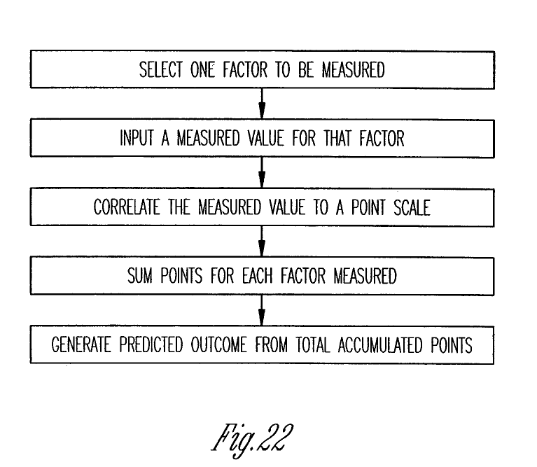

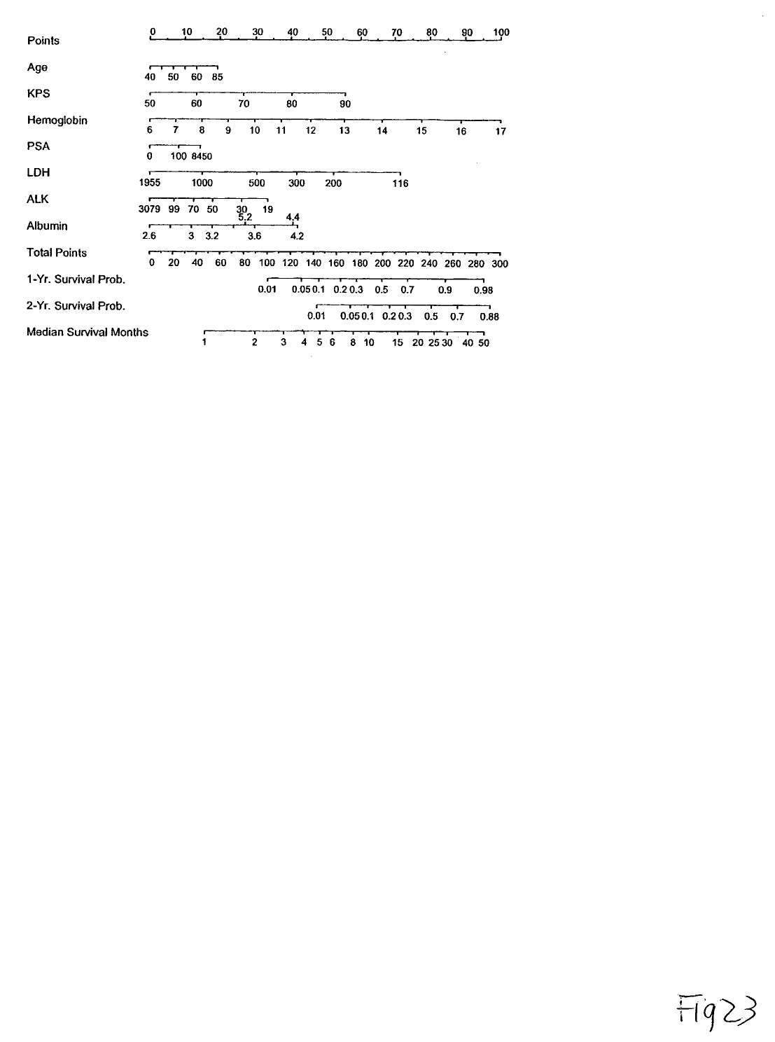

To provide a nomogram useful to predict progression to death in patients with metastases at the time of primary or subsequent therapy, serum markers may be employed with factors such as Karnofsky performance status, hemoglobin, PSA, lactate dehydrogenase, alkaline phosphatase and albumin to predict time to death including median, 1 year and 2 year survival (FIG. 22). In one embodiment, the nomogram is employed to predict time to death in patients with hormone sensitive prostate cancer. In another embodiment, the nomogram is employed to predict time to death in patients with hormone refractory disease. In one embodiment, one or more of TGF-β₁, IL6sR, IL6, VEGF, sVCAM, UPA or UPAR levels or amounts are employed with Karnofsky performance status, hemoglobin, PSA, lactate dehydrogenase, alkaline phosphatase and albumin. In another embodiment, one or more of TGF-β₁, IL6sR, IL6, VEGF, sVCAM, UPA or UPAR levels or amounts are employed in place of one or more of Karnofsky performance status, hemoglobin, PSA, lactate dehydrogenase, alkaline phosphatase and albumin.

### REFERENCES

Adams et al., Cancer Res., 60:2898 (2000).

Adler et al., J. Urol., 161:182 (1999).

Ali et al., Urology, 57:143 (2001).

Benjamin et al., J. Clin. Invest., 103:159 (1999).

Blann et al. Thromb. Haemost., 79:1080 (1998).

Bostwick et al., Urology, 48:47 (1996).

Breiman et al., Classification and Regression Trees. Monterey, Calif., Wadsworth and Brooks/Cole (1984).

Byrne et al., J. Natl. Cancer Inst., 92:1329 (2000).

Catalona et al., J. Urol., 160:2428 (1998).

Chang et al., J. Urol., 160:2111 (1998).

Chevalier et al., Mol. Cell Endocrinol., 189:169 (2002).

Coalo, J. Clin. Endocrinol. Metab., 83:775 (1998).

Cooper et al., Clin. Exp. Metastasis, 19:25 (2002).

Cowen et al., Med. Decis. Making, 18:376 (1998).

Cox, J. Royal Statistical Society, B34, 187-220 (1972)

Cristianni et al., An Introduction to Support Vector Machines, Cambridge University Press (2000).

Cupp et al., J. Urol., 153:1543 (1995).

D'Amico et al., J. Clin. Oncol., 17:168 (1999).

D'Amico et al., J. Clin. Oncol., 18:1164 (2000).

D'Amico et al., JAMA, 280:969 (1998).

Danielpour et al., Annals New York Academy of Sciences, pp. 300-302 (1990).

Danielpour et al., J. Cell. Phy., 138:79 (1989).

Danielpour, J. Immunol. Meth., 158: 17 (1993).

Dasch et al., Annals New York Academy of Sciences, pp. 303-305 (1990).

De Caterina et al., Arterioscler. Thromb. Vasc. Biol., 17:2646 (1997).

de Lemos et al., J. Am. Coll. Cardiol., 36:423 (2000).

Dorey et al., Stats. in Med., 12:1589 (1993).

Duque et al., Urology, 54:523 (1999).

Eastham et al., In: N J Vogelzang, P T Scardino, W U Shipley, and D S Coffey (eds), Comprehensive textbook of Genitourinary Oncology, pp 722-738 (2000).

Eastham et al., Lab. Invest., 73:628 (1995).

Eastham et al., Semin. Urol. Oncol., 20:108 (2002).

Eder et al., J. Urol., 156: 953 (1996).

Efron B, Tibshirani R J. An introduction to the bootstrap. New York: Chapman & Hall; 1993.

Egawa et al., Urology, 52:433 (1998).

Engeler et al., Urology, 40:346 (1992).

Epstein et al., Am. J. Surg. Pathol., 20:286 (1996).

Epstein et al., J. Urol., 149:1478 (1993).

Epstein et al., J. Urol., 160:2407 (1998).

Epstein et al., J. Urol., 160:97 (1998).

Epstein et al., JAMA, 271:368 (1994).

Epstein et al., Urology, 56:283 (2000).

Farkas et al., Urology, 52:444 (1998).

Ferrer et al., Urology, 51:161 (1998).

Flanders, J. Cell Biol., 108:653 (1989).

Galizia et al., Clin. Immunol., 102:169 (2002).

Garcia et al., Mod. Pathol., 13:717 (2000).

George et al., Clin. Cancer Res., 7:1932 (2001).

Gilliland et al., J. Urol., 162:1341 (1999). Giri et al., Am. J. Path., 159:2159 (2001).

Gore et al., J. Urol., 165:1554 (2001).

Goto et al., Int. J. Urol., 5:337 (1998).

Graefen et al., J. Clin. Oncol., 20:3206 (2002).

Graefen et al., Urologic Oncology, 7:141 (2002).

Greene et al., Br. J. Urol., 68:499 (1991).

Greene et al., Campbell's Urology, vol. 1, 6th ed, W. B. Saunders Co. (1992).

Greene et al., J. Urol., 146:1069 (1991).

Grossklaus et al., J. Urol., 167:2032 (2002).

Harrell et al., JAMA, 247:2543 (1982).

Harrell et al., Stat. Med., 15:361 (1996).

Harrell, In: Regression Modeling Strategies with Applications to Linear Models, Logistic Regression, and Survival Analysis, Springer-Verlag, NY (2001).

Hastie, The Elements of Statistical Learning, Springer (2001).

Heinrich et al., Biochem. J., 334:297 (1998).

Hirano, Int. Rev. Immunol., 16:249 (1998).

Hirano et al., Oncology, 19:2548 (2000).

Hobisch et al., J. Pathol., 191:239 (2000).

Hodge et al., J. Urol., 142:71 (1989).

Huss et al., Cancer Res., 61:2736 (2001). Ivanovic et al., Nat. Med., 1: 282 (1995).

Jones et al., BJU Int., 85:276 (2000).

Jones, J. Urol., 144:89 (1990).

Junker et al., Cytokine, 8:794 (1996).

Kakehi et al., Urol. Oncol., 2:131 (1996).

Kattan et al., Cancer, 79:528 (1997).

Kattan et al., J. Clin. Oncol., 18:3352 (2000).

Kattan et al., Int. J. Radiol. Oncol. Biol. Phys., 48:1469 (2000).

Kattan et al., J. Gen. Intern. Med., 12:299 (1997).

Kattan et al., Urology, 58:393 (2001).

Kattan et al., J. Clin. Oncol., 17:1499 (1999).

Kattan, J. Natl. Cancer Inst., 90:766 (1998).

Koch et al., J. Urol., 164:749 (2000).

Kong et al., Cancer, 86:1712 (1999).

Kong et al., Ann. Surg., 222:155 (1995).

Kuniyasu et al., Clin. Cancer Res., 6:2295 (2000).

Langley et al., Microcirculation, 8:335 (2001).

Leventis et al., J. Clin. Oncol., 19:1030 (2001).

Liu et al., BBRC, 290:249 (2000).

Lucas et al., J. Immunol., 145:1415 (1990).

Lynch et al., Prostate, 32:214 (1997).

Macleod et al., J. Pathol., 187:43 (1999).

Mai et al., Urol. Int., 66:191 (2001).

Maru et al., J. Urol., 161:1280 (1999).

Mehta et al., Clin. Cancer Res., 7:81 (2001).

Merrill et al., J. Urol., 163:503 (2000).

Miller, Breast Cancer Res. Treat, 75:S45 (2002).

Mullberg et al., J. Immunol., 152:4958 (1994).

Nakashima et al., Clin. Cancer Res., 6:2702 (2000).

Narain et al., Prostate, 49:185 (2001).

Newcomer et al., J. Urol., 158:1427 (1997).

Noguchi et al., J. Urol., 164:1596 (2000).

Noguchi et al., J. Urol., 166:104 (2001).

Nunn et al., J. Cell Physiol., 171:196 (1997).

Oesterling et al., JAMA, 269:57 (1993).

Oh et al., Cytokine, 8:401 (1996).

Ohori et al., Am. J. Surg. Pathol., 17:1252 (1993).

Obori et al., Cancer, 74:104 (1994).

Ohori et al., J. Urol., 154:1818 (1995).

Ohori et al., Prostate, 23:271 (1993).

Partin et al., J. Urol., 150:110 (1993).

Partin et al., JAMA, 277:1445 (1997).

Partin et al., JAMA, 278:118 (1997).

Partin et al., Urol. Clin. North Am., 20:713 (1993).

Partin et al., Urol. Clin. North Am., 20:713 (1994).

Partin et al., Urology, 58:843 (2001).

Perry et al., Urology, 49:151 (1997).

Peter et al., Thromb. Haemost., 82:38 (1999).

Peters et al., Blood, 92:3495 (1998).

Pisany et al., Int. J. Radial. Oncol. Biophys., 36:585 (1996).

Pound et al., JAMA, 281:1591 (1999). Presti et al., J. Urol., 169:125 (2003).

Roberts et al., Mayo Clin. Proc., 76:576 (2001).

Rosen et al., J. Urol., 148:331 (1992).

Rumelhart et al., (eds), Parallel Distributed Processing: Exploration in the Microstructure of Cognition Volume 1, Foundations Cambridge, Mass, The MIT Press (1986).

San Francisco et al., J. Urol., 169:136 (2003).

Schmid et al., Cancer, 71:2031 (1993).

Sebo et al., Cancer, 91:2196 (2001).

Sebo et al., J. Urol., 163:174 (2000).

Shariat et al., Cancer Research, In Press.

Shariat et al., J. Clin. Oncol., 19:2856 (2001c).

Shariat et al., J. Clin. Oncol., 20:833 (2002).

Shariat et al., J. Clin. Oncol.,

Shariat et al., Urology, 58:1008 (2001b).

Shipley et al., JAMA, 281:1598 (1995).

Shirai et al., Cancer, 73:2275 (1994).

Silberman et al., Cancer, 79:772 (1997).

Simiantonaki et al., Exp. Mol. Pathol., 73:46 (2002).

Smaletz et al., J. Clin. Oncol., 20:3972 (2002).

Smith et al., JAMA, 276:1309 (1996). Spence et al., Int. J. Biol. Markers, 17:119 (2002).

Sridhara et al., J. Clin. Oncol., 13:2944 (1995).

Stamey et al., J. Urol., 139:1235 (1998).

Stamey et al., J. Urol., 167:103 (2002).

Stamey, Clin. Chem., 47:631 (2001).

Stapleton et al., Cancer, 82:168 (1998).

Stein et al., J. Urol., 147:942 (1992).

Steiner et al., Mol Endocrinol., 6:15 (1992).

Sutkowski et al., J. Natl. Cancer Inst., 91:1663 (1991).

Tamura et al., PNAS USA, 90:11924 (1993).

Taylor et al., Urology, 60:841 (2002).

Tefilli et al., J. Urol., 160:802 (1998).

Thompson et al., J. Cell Biochem. Suppl., 54-61 (1992).

Thompson et al., J. Cell Biol., 108:661 (1989).

Truong et al., Hum. Pathol., 24:4 (1993).

Tsushima et al., Clin. Cancer Res., 7:1258 (2001).

Volavsek et al., Pathol. Oncol. Res., 6:191 (2000).

Wakefield et al., Clin. Cancer Res., 1:129 (1995).

Walsh et al., J. Urol., 152:1831 (1994).

Wei et al., J. Clin. One., 20:557 (2002).

Weidner et al., Am. J. Pathol., 143: 401 (1993).

Weidner et al., N. Engl. J. Med., 324:1 (1991).

Wheeler et al., Hum. Pathol., 29:856 (1998).

Wheeler et al., Hum. Pathol., 29:856 (1998).

Wheeler et al., Prostate, 25:274 (1994).

Wheeler et al., Prostate, 25:274 (1994).

Wheeler, Urol. Clin. North Am., 16:623 (1989).

Wikstrom et al., Prostate, 51:268 (2002).

Wills et al., Urology, 51:759 (1998).

Wolff et al., Anticancer Res., 19:2657 (1999).

Yang et al., J. Urol., 159:941 (1998)

Zagars et al., Cancer, 79:1370 (1997).

Zetter, Semin. Cancer Biol., 4:219 (1993).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to determine the risk of progression of a prostate cancer patient after therapy, comprising:
a) detecting or determining the amount or level of VEGF, UPAR, UPA, or sVCAM in a blood sample obtained from a patient prior to therapy for clinically localized prostate cancer; and
b) correlating the amount or level of VEGF, UPAR, UPA, or sVCAM with the risk of progression.

2. A method to determine the risk of progression of a prostate cancer patient after therapy, comprising:
a) detecting or determining the amount or level of TGF-β₁ and IL6sR or IL6 in a blood sample, and the Gleason score in a prostate sample, obtained from a patient prior to therapy for clinically localized prostate cancer; and
b) correlating the amount or level of TGF-β₁ and IL6sR or IL6 and the Gleason score in a prostate sample, with the risk of progression.

3. A method to determine the prognosis of a prostate cancer patient after therapy, comprising:
a) detecting or determining the amount or level of TGF-β₁ and IL6sR or IL6 in a blood sample, and the Gleason score in a prostate sample, obtained from a patient prior to therapy for clinically localized prostate cancer; and
b) correlating the amount or level of TGF-β₁ and IL6sR or IL6 and the Gleason score in a prostate sample with the risk of non-prostate confined disease.

4. The method of claim 1, 2, or 3 wherein the clinical stage of the patient is T3a, T3, T2c, T2b, T2a, T2, T1c, T1b, T1a or T1.

5. The method of claim 1, 2, or 3 wherein the therapy is primary therapy.

6. The method of claim 1, 2, or 3 wherein the therapy is surgery, radical prostatectomy, radiation therapy or a radioactive seed implant.

7. The method of claim 1, 2, or 3 wherein the patient has not been subject to hormonal therapy.

8. The method of claim 1 wherein the amount or level of VEGF is detected or determined with an agent that binds to VEGF.

9. The method of claim 8 wherein the agent is an antibody.

10. The method of claim 9 wherein the agent is detectably labeled or binds to a detectable label.

11. The method of claim 1 wherein the amount of level of sVCAM is detected or determined with an agent that binds sVCAM.

12. The method of claim 11 wherein the agent is an antibody.

13. The method of claim 12 wherein the agent is detectably labeled or binds to a detectable label.

14. The method of claim 1 wherein the amount or level of UPAR is detected or determined with an agent that binds to UPAR.

15. The method of claim 14 wherein the agent is an antibody.

16. The method of claim 15 wherein the agent is detectably labeled or binds to a detectable label.

17. The method of claim 1 wherein the amount or level of UPA is detected or determined with an agent that binds to UPA.

18. The method of claim 17 wherein the agent is an antibody.

19. The method of claim 18 wherein the agent is detectably labeled or binds to a detectable label.

20. The method of claim 2 or 3 wherein the amount of level of TGF-β₁ is detected or determined with an agent that binds TGF-β₁.

21. The method of claim 20 wherein the agent is an antibody.

22. The method of claim 21 wherein the agent is detectably labeled or binds to a detectable label.

23. The method of claim 2 or 3 wherein the amount of level of IL6sR or IL6 is detected or determined with an agent that binds IL6sR or IL6.

24. The method of claim 23 wherein the agent is an antibody.

25. The method of claim 24 wherein the agent is detectably labeled or binds to a detectable label.

26. The method of claim 1, 2 or 3 wherein the correlating is conducted by a computer.

27. The method of claim 2 or 3 wherein the blood plasma sample is a platelet poor plasma sample.

28. The method of claim 1 wherein the blood sample is a plasma sample.

29. The method of claim 2 or 3 further comprising detecting or determining a second Gleason score.

30. The method of claim 2 or 3 further comprising detecting or determining clinical stage.

31. An apparatus, comprising:
a data input means, for input of test information comprising the level or amount of VEGF, UPAR, UPA, or sVCAM, in one or more samples obtained from a mammal;
a processor, executing a software for analysis of the level or amount of VEGF, UPAR, UPA, or sVCAM, in the one or more samples;
wherein the software analyzes the level or amount of VEGF, UPAR, UPA, or sVCAM, in the one or more samples and provides the risk of prostate disease progression in the mammal.

32. An apparatus, comprising:
a data input means, for input of test information comprising the level or amount of UPAR or UPA in one or more samples obtained from a mammal;
a processor, executing a software for analysis of the level or amount of UPAR or UPA in the one or more samples;
wherein the software analyzes the level or amount of UPAR or UPA in one or more samples and provides the risk of non-prostate confined disease in the mammal.

33. An apparatus, comprising:
a data input means, for input of test information comprising the level or amount of TGF-β₁ and IL6sR or IL6, and the Gleason score, in one or more samples obtained from a mammal;
a processor, executing a software for analysis of the level or amount of TGF-β₁ and IL6sR or IL6, and the Gleason score, in the one or more samples;
wherein the software analyzes the level or amount of TGF-β₁ and IL6sR or IL6, and the Gleason score, in the one or more samples and provides the risk of prostate disease progression in the mammal.

34. An apparatus, comprising:
a data input means, for input of test information comprising the level or amount of TGF-β₁ and IL6sR or IL6, and the Gleason score, in one or more samples obtained from a mammal;
a processor, executing a software for analysis of the level or amount of TGF-β₁ and IL6sR or IL6, and the Gleason score, in the one or more samples;
wherein the software analyzes the level or amount of TGF-β₁ and IL6sR or IL6, and the Gleason score, in one or more samples and provides the risk of non-prostate confined disease in the mammal.

35. The apparatus of claim 31, 32, 33 or 34 wherein the amount or level and score is input manually using the data input means.

36. The apparatus of claim 31, 32, 33 or 34 wherein the software constructs a database of the test information.

37. The apparatus of claim 33 or 34 wherein the information further comprises a second Gleason score.

38. The apparatus of claim 33 or 34 wherein the information further comprises clinical grade.

39. A method to determine the prognosis of a prostate cancer patient after therapy, comprising:
a) inputting test information to a data input means, wherein the information comprises the level or amount of VEGF, UPAR, UPA, or sVCAM, in one or more samples obtained from a prostate cancer patient;
b) executing a software for analysis of the test information; and
c) analyzing the test information so as to provide the risk of disease progression or non-prostate confined disease in the patient.

40. A method to determine the prognosis of a prostate cancer patient after therapy, comprising:
a) inputting test information to a data input means, wherein the information comprises the level or amount of TGF-β₁ and IL6sR or IL6, and the Gleason score, in one or more samples obtained from a prostate cancer patient;
b) executing a software for analysis of the test information; and
c) analyzing the test information so as to provide the risk of disease progression or non-prostate confined disease in the patient.

41. A method for predicting a probability of recurrence of prostatic cancer in a patient following radical prostatectomy, comprising:
a) correlating a set of pre-operative factors for the patient to a functional representation of a set of pre-operative factors determined for each of a plurality of persons previously diagnosed with prostatic cancer and having been treated by radical prostatectomy, so as to yield a value for total points for the patient, which set of factors for each of a plurality of persons is correlated with the incidence of recurrence of prostatic cancer for each person in the plurality of persons, wherein the set of pre-operative factors comprises pre-treatment TGF-β₁ level, pre-treatment IL6sR or IL6 level, and optionally one or more of pre-treatment PSA level, primary Gleason grade or secondary Gleason grade, wherein the functional representation comprises a scale for each of pre-treatment TGF-β₁ level, pre-treatment IL6sR or IL6 level, and optionally a scale for one or more of pre-treatment PSA level, primary Gleason grade or secondary Gleason grade, a points scale, a total points scale, and a predictor scale, wherein the scales for pre-treatment TGF-β₁ level, pre-treatment IL6sR or IL6 level, and optionally one or more of pre-treatment PSA level, primary Gleason grade or secondary Gleason grade, each have values on the scales which can be correlated with values on the points scale, and wherein the total points scale has values which may be correlated with values on the predictor scale; and
b) correlating the value on the total points scale for the patient with a value on the predictor scale to predict the quantitative probability of recurrence of prostatic cancer in the patient following radical prostatectomy.

42. The method of claim 41 wherein the functional representation is a nomogram.

43. The method of claim 42 wherein the nomogram is generated with a Cox proportional hazards regression model.

44. The method of claim 41 wherein the patient is a presurgical candidate.

45. The method of claim 41 wherein the probability of recurrence of prostatic cancer is a probability of remaining free of prostatic cancer five years following radical prostatectomy.

46. The method of claim 41 wherein a recurrence of prostatic cancer is characterized as an increased serum PSA level.

47. The method of claim 46 wherein the increased serum PSA level is greater than or equal to 0.2 ng/mL.

48. The method of claim 41 wherein a recurrence of prostatic cancer is characterized as a positive biopsy, bone scan or the application of further treatment for prostate cancer because of the high probability of subsequent recurrence of the cancer.

49. The method of claim 41 wherein the plurality of persons comprises persons with clinically localized prostate cancer not treated previously by radiotherapy, hormone therapy or cryotherapy, and subsequently undergoing radical prostatectomy.

50. The method of claim 41 wherein the set of pre-operative factors further comprise clinical stage, pre-treatment VEGF level, pre-treatment sVCAM level, pre-treatment UPAR level, or pre-treatment UPA level.

51. An apparatus for predicting a probability of disease recurrence in a patient with prostatic cancer following a radical prostatectomy, which apparatus comprises:
a) a correlation of a set of pre-operative factors for each of a plurality of persons previously diagnosed with prostatic cancer and having been treated by radical prostatectomy with the incidence of recurrence of prostatic cancer for each person of the plurality of persons, wherein the set of pre-operative factors comprises pre-treatment TGF-β₁ level, pre-treatment IL6sR or IL6 level, and optionally one or more of pre-treatment PSA level, primary Gleason grade or secondary Gleason grade; and
b) a means for comparing an identical set of pre-operative factors determined from a patient diagnosed as having prostatic cancer to the correlation to predict the quantitative probability of recurrence of prostatic cancer in the patient following radical prostatectomy.

52. A nomogram for the graphic representation of a quantitative probability that a patient with prostate cancer will remain free of disease following radical prostatectomy, comprising: a plurality of scales and a solid support, the plurality of scales being disposed on the support and comprising a scale for each of pre-treatment TGF-β₁ level, pre-treatment IL6sR or IL6 level, and optionally one or more of pre-treatment PSA level, primary Gleason grade or secondary Gleason grade, a points scale, a total points scale and a predictor scale, wherein the scales for pre-treatment TGF-β₁ level, pre-treatment IL6sR or IL6 level, and optionally the scales for one or more of the pre-treatment PSA level, primary Gleason grade or secondary Gleason grade each has values on the scales, and wherein the scales for pre-treatment TGF-β₁ level, pre-treatment IL6sR or IL6 level, and optionally the scales for one or more of pre-treatment PSA level, primary Gleason grade or secondary Gleason grade are disposed on the solid support with respect to the points scale so that each of the values on the pre-treatment TGF-β₁ level, pre-treatment IL6sR or IL6 level, and optionally the one or more of the pre-treatment PSA level, primary Gleason grade or secondary Gleason grade can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's pre-treatment TGF-β₁ level, pre-treatment IL6sR or IL6 level, and optionally one or more of pre-treatment PSA level, primary Gleason grade or secondary Gleason grade can be added together to yield a total points value, and the total points value can be correlated with the predictor scale to predict the quantitative probability of recurrence.

53. The nomogram of claim 52 wherein the solid support is a laminated card.

54. A method to predict a pre-operative prognosis in a patient comprising:
determining a set of pre-operative factors for a patient, which set comprises pre-treatment TGF-β₁ level, pre-treatment IL6sR or IL6 level, and optionally one or more of pre-treatment PSA level, primary Gleason grade or secondary Gleason grade; matching the pre-operative factors to the values on the scales of the nomogram of claim 52; determining a separate point value for each of the pre-operative factors; adding the separate point values together to yield a total points value; and
correlating the total points value with a value on the predictor scale of the nomogram to determine the pre-operative prognosis of the patient.

55. An apparatus for predicting a probability of disease recurrence in a patient with prostatic cancer following a radical prostatectomy, which apparatus comprises: a scale for each of pre-treatment TGF-β₁ level, pre-treatment IL6sR or IL6 level, and optionally one or more of pre-treatment PSA level, primary Gleason grade or secondary Gleason grade, a points scale, a total points scale and a predictor scale, wherein the scales for pre-treatment TGF-β₁ level, pre-treatment IL6sR or IL6 level, and optionally the scales for one or more of pre-treatment PSA level, primary Gleason grade or secondary Gleason grade each has values on the scales, and wherein the scales for pre-treatment TGF-β₁ level, pre-treatment IL6sR or IL6 level, and optionally the scales for one or more of pre-treatment PSA level, primary Gleason grade or secondary Gleason grade are disposed so that each of the values on the pre-treatment TGF-β₁ level, pre-treatment IL6sR or IL6 level, and optionally the one or more of the pre-treatment PSA level, primary Gleason grade or secondary Gleason grade, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's pre-treatment TGF-β₁ level, pre-treatment IL6sR or IL6 level, and optionally one or more of pre-treatment PSA level, primary Gleason grade or secondary Gleason grade can be added together to yield a total points value, and the total points value can be correlated with the predictor scale to predict the quantitative probability of recurrence.

56. A method to determine the risk of progression of a prostate cancer patient after therapy, comprising:
a) providing i) the amount or level of TGF-β₁ in a blood plasma sample obtained from the patient after therapy; ii) pathological Gleason score; and iii) and optionally the amount or level of one or more of IL6sR, IL6 or PSA in a blood sample obtained from the patient prior to therapy; and
b) correlating the amount or level of post-treatment TGF-β₁, pathological Gleason score and optionally the amount or level of one or more of pre-treatment IL6sR, IL6 or PSA, with the risk of progression.

57. An apparatus, comprising:
a data input means, for input of test information comprising the level or amount of post-treatment TGF-β₁, pathological Gleason score, and optionally level or amount of one or more of pre-treatment IL6sR, IL6 or PSA, in one or more samples obtained from a mammal;
a processor, executing a software for analysis of the level or amount of post-treatment TGF-β₁, pathological Gleason score, and optionally level or amount of one or more of pre-treatment IL6sR, IL6 or PSA in the one or more samples;
wherein the software analyzes the level or amount of post-treatment TGF-β₁, pathological Gleason score, and optionally level or amount of one or more of pre-treatment IL6sR, IL6 or PSA in the one or more samples and provides the risk of prostate disease progression in the mammal.

58. A method to determine the prognosis of a prostate cancer patient after therapy, comprising:
a) inputting test information to a data input means, wherein the information comprises the level or amount of post-treatment TGF-β₁, pathological Gleason score, and optionally level or amount of one or more of pre-treatment IL6sR, IL6 or PSA, samples obtained from a prostate cancer patient;
b) executing a software for analysis of the test information; and
c) analyzing the test information so as to provide the risk of disease progression or non-prostate confined disease in the patient.

59. A method for predicting a probability of recurrence of prostatic cancer in a patient following radical prostatectomy, comprising:
a) correlating a set of factors for the patient to a functional representation of a set of factors determined for each of a plurality of persons previously diagnosed with prostatic cancer and having been treated by radical prostatectomy so as to yield a value for total points for the patient, which set of factors for each of a plurality of persons is correlated with the incidence of recurrence of prostatic cancer for each person in the plurality of persons, wherein the set of factors comprises post-treatment TGF-β₁ level, pathological Gleason score and optionally one or more of pre-treatment IL6sR, IL6 or PSA level, wherein the functional representation comprises a scale for each of post-treatment TGF-β₁ level, pathological Gleason score, and optionally one or more of pre-treatment IL6sR or IL6 or PSA level, a points scale, a total points scale, and a predictor scale, wherein the scales for post-treatment TGF-β₁ level, pathological Gleason score, and optionally one or more of pre-treatment IL6sR, IL6 or PSA level each have values on the scales which can be correlated with values on the points scale, and wherein the total points scale has values which may be correlated with values on the predictor scale; and
b) correlating the value on the total points scale for the patient with a value on the predictor scale to predict the quantitative probability of recurrence of prostatic cancer in the patient following radical prostatectomy.

60. An apparatus for predicting a probability of disease recurrence in a patient with prostatic cancer following a radical prostatectomy, which apparatus comprises:
a) a correlation of a set of factors for each of a plurality of persons previously diagnosed with prostatic cancer and having been treated by radical prostatectomy with the incidence of recurrence of prostatic cancer for each person of the plurality of persons, wherein the set of factors comprises post-treatment TGF-β₁ level, pathological Gleason, and optionally one or more of pre-treatment IL6sR, IL6 or PSA level; and
b) a means for comparing an identical set of factors determined from a patient diagnosed as having prostatic cancer to the correlation to predict the quantitative probability of recurrence of prostatic cancer in the patient following radical prostatectomy.

61. A nomogram for the graphic representation of a quantitative probability that a patient with prostate cancer will remain free of disease following radical prostatectomy, comprising: a plurality of scales and a solid support, the plurality of scales being disposed on the support and comprising a scale for each of post-treatment TGF-β₁ level, pathological Gleason score and optionally one or more of pre-treatment IL6sR, IL6 or PSA level, a points scale, a total points scale and a predictor scale, wherein the scales for post-treatment TGF-β₁ level, pathological Gleason score, and optionally one or more of pre-treatment IL6sR, IL6 or PSA level, each has values on the scales, and wherein the scales for post-treatment TGF-β₁ level, pathological Gleason score and optionally one or more of pre-treatment IL6sR, IL6 or PSA level are disposed on the solid support with respect to the points scale so that each of the values on the post-treatment TGF-β₁ level, pathological Gleason score and optionally one or more of pre-treatment IL6sR, IL6 or PSA level can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the patient's post-treatment TGF-β₁ level, pathological Gleason score and optionally one or more of pre-treatment IL6sR, IL6 or PSA level, can be added together to yield a total points value, and the total points value can be correlated with the predictor scale to predict the quantitative probability of recurrence.

62. A method to predict a post-operative prognosis in a patient comprising: determining a set of factors for a patient which set comprises post-treatment TGF-β₁ level, Gleason score and optionally one or more of pre-treatment IL6sR, IL6 or PSA level, matching the pre-operative factors to the values on the scales of the nomogram of claim 61; determining a separate point value for each of the factors; adding the separate point values together to yield a total points value; and correlating the total points value with a value on the predictor scale of the nomogram to determine the post-operative prognosis of the patient.

63. An apparatus for predicting a probability of disease recurrence in a patient with prostatic cancer following a radical prostatectomy, which apparatus comprises: a scale for each of post-treatment TGF-β₁ level, pathological Gleason score and optionally one or more of pre-treatment IL6sR, IL6 or PSA level, a points scale, a total points scale and a predictor scale, wherein the scales for post-treatment TGF-β₁ level, pathological Gleason score, and optionally one or more of pre-treatment IL6sR, IL6 or PSA level, each has values on the scales, and wherein the scales for post-treatment TGF-β₁ level, pathological Gleason score and optionally one or more of pre-treatment IL6sR, IL6 or PSA level are disposed with respect to the points scale so that each of the values on the post-treatment TGF-β₁ level, pathological Gleason score and optionally one or more of pre-treatment IL6sR, IL6 or PSA level can be correlated with values on the points scale, wherein the total points scale has values on the total points scale, and wherein the total points scale is disposed with respect to the predictor scale so that the values on the total points scale may be correlated with values on the predictor scale, such that the values on the points scale correlating with the patient's post-treatment TGF-β₁ level, pathological Gleason score and optionally one or more of pre-treatment IL6sR, IL6 or PSA level, can be added together to yield a total points value, and the total points value can be correlated with the predictor scale to predict the quantitative probability of recurrence.

64. The method of claim 1, 2, 31, 39, 40, 41, 54, 56, 58, 59 or 62 further comprising further correlating one of Gleason score, number of positive cores, number of positive contiguous cores, total cancer length, total cancer in contiguous cores and/or percent tumor involvement from a systemic 12 core biopsy to the risk of progression or non-prostate confined disease.

65. The apparatus of claim 31, 32, 33, 34, 51, 55, 57, 60 or 63 further comprising correlating one of Gleason score, number of positive cores, number of positive contiguous cores, total cancer length, total cancer in contiguous cores and/or percent tumor involvement from a systemic 12 core biopsy to the risk of progression or non-prostate confined disease.

66. The nomogram of claim 52 or 61 further comprising correlating one of Gleason score, number of positive cores, number of positive contiguous cores, total cancer length, total cancer in contiguous cores and/or percent tumor involvement from a systemic 12 core biopsy to the risk of progression or non-prostate confined disease from a systemic 12 core biopsy to predict the quantitative probability of recurrence.

67. A method to determine the risk of progression of a prostate cancer patient after therapy, comprising:
a) providing i) the amount or level of TGF-β₁ in a blood plasma sample obtained from a patient prior to therapy; ii) the amount or level of IL6sR or IL6 in a blood sample obtained from a patient prior to therapy; and iii) the Gleason score in a prostate sample; and
b) correlating the amount or level of TGF-β₁ and IL6sR or IL6 and the Gleason score in a prostate sample with the risk of non-prostate confined disease.
